SEQUENCE LISTING (141 sequences)

SEQ ID NO: 1 - DNA, 21 bp, Artificial Sequence
  Description of Artificial Sequence Synthetic primer
gatggaatcttgctggtgcat

SEQ ID NO: 2 - DNA, 32 bp, Artificial Sequence
  Description of Artificial Sequence Synthetic probe
cccaatctacagctgtctttactgaatcaggc

SEQ ID NO: 3 - DNA, 24 bp, Artificial Sequence
  Description of Artificial Sequence Synthetic primer
ttatggcttctaactgcatctctt

SEQ ID NO: 4 - DNA, 20 bp, Artificial Sequence
  Description of Artificial Sequence Synthetic primer
atggaatcttgctggtgcat

SEQ ID NO: 5 - DNA, 20 bp, Artificial Sequence
  Description of Artificial Sequence Synthetic primer
tggcttctaactgcatctct

SEQ ID NO: 6 - DNA, 28 bp, Artificial Sequence
  Description of Artificial Sequence Synthetic primer
gctatattgaaaaatattggtggagtct

SEQ ID NO: 7 - DNA, 21 bp, Artificial Sequence
  Description of Artificial Sequence Synthetic primer
tggcttctaactgcatctctt

SEQ ID NO: 8 - DNA, 28 bp, Artificial Sequence
  Description of Artificial Sequence Synthetic primer
aatattggtggagtctatctagatgttg

SEQ ID NO: 9 - DNA, 25 bp, Artificial Sequence
  Description of Artificial Sequence Synthetic primer
attatggcttctaactgcatctctt

SEQ ID NO: 10 - DNA, 26 bp, Artificial Sequence
  Description of Artificial Sequence Synthetic primer
tattggtggagtctatctagatgttg

SEQ ID NO: 11 - DNA, 23 bp, Artificial Sequence
  Description of Artificial Sequence Synthetic primer
tatggcttctaactgcatctctt

SEQ ID NO: 12 - DNA, 23 bp, Artificial Sequence
  Description of Artificial Sequence Synthetic primer
tggtggagtctatctagatgttg

SEQ ID NO: 13 - DNA, 27 bp, Artificial Sequence
  Description of Artificial Sequence Synthetic primer
cagaatatacctcagaacattttgaca

SEQ ID NO: 14 - DNA, 29 bp, Artificial Sequence
  Description of Artificial Sequence Synthetic probe
tctagtggtgatgcctccatatcaccaag

SEQ ID NO: 15 - DNA, 27 bp, Artificial Sequence
  Description of Artificial Sequence Synthetic primer
agcccttgatttataatacccttacta

SEQ ID NO: 16 - DNA, 29 bp, Artificial Sequence
  Description of Artificial Sequence Synthetic probe
ctagtggtgatgcctccatatcaccaagt

SEQ ID NO: 17 - DNA, 24 bp, Artificial Sequence
  Description of Artificial Sequence Synthetic primer
ctttcacagaaattagcccttgat

SEQ ID NO: 18 - DNA, 26 bp, Artificial Sequence
  Description of Artificial Sequence Synthetic primer
gaacattttgacatgttagacgaaga

SEQ ID NO: 19 - DNA, 28 bp, Artificial Sequence
  Description of Artificial Sequence Synthetic probe
agtggtgatgcctccatatcaccaagtg

SEQ ID NO: 20 - DNA, 26 bp, Artificial Sequence
  Description of Artificial Sequence Synthetic primer
agcccttgatttataatacccttact

SEQ ID NO: 21 - DNA, 25 bp, Artificial Sequence
  Description of Artificial Sequence Synthetic primer
tgacatgttagacgaagaagttcaa

SEQ ID NO: 22 - DNA, 32 bp, Artificial Sequence
  Description of Artificial Sequence Synthetic probe
tgcaattttaacttctagtggtgatgcctcca

SEQ ID NO: 23 - DNA, 25 bp, Artificial Sequence
  Description of Artificial Sequence Synthetic primer
acatgttagacgaagaagttcaaag

SEQ ID NO: 24 - DNA, 25 bp, Artificial Sequence
  Description of Artificial Sequence Synthetic primer
catgttagacgaagaagttcaaagt

SEQ ID NO: 25 - DNA, 25 bp, Artificial Sequence
  Description of Artificial Sequence Synthetic primer
ctttcacagaaattagcccttgatt

SEQ ID NO: 26 - DNA, 27 bp, Artificial Sequence
  Description of Artificial Sequence Synthetic primer
agtagttttgaatctgttctagcttct

SEQ ID NO: 27 - DNA, 29 bp, Artificial Sequence
  Description of Artificial Sequence Synthetic probe
tagtggtgatgcctccatatcaccaagtg

SEQ ID NO: 28 - DNA, 25 bp, Artificial Sequence
  Description of Artificial Sequence Synthetic primer
tgaatctgttctagcttctaagtca

SEQ ID NO: 29 - DNA, 28 bp, Artificial Sequence
  Description of Artificial Sequence Synthetic probe
cacttggtgatatggaggcatcaccact

SEQ ID NO: 30 - DNA, 29 bp, Artificial Sequence
  Description of Artificial Sequence Synthetic primer
actttaggtccaattattagtcaaggtaa

SEQ ID NO: 31 - DNA, 35 bp, Artificial Sequence
  Description of Artificial Sequence Synthetic probe
agctcccaaactttcaccaaaattgttcattgtag

SEQ ID NO: 32 - DNA, 24 bp, Artificial Sequence
  Description of Artificial Sequence Synthetic primer
ccaacccttaaataacttccgatt

SEQ ID NO: 33 - DNA, 27 bp, Artificial Sequence
  Description of Artificial Sequence Synthetic primer
caacccttaaataacttccgatttttg

SEQ ID NO: 34 - DNA, 28 bp, Artificial Sequence
  Description of Artificial Sequence Synthetic primer
cttcaggataaaatccaacccttaaata

SEQ ID NO: 35 - DNA, 29 bp, Artificial Sequence
  Description of Artificial Sequence Synthetic primer
tttaggtccaattattagtcaaggtaatg

SEQ ID NO: 36 - DNA, 29 bp, Artificial Sequence
  Description of Artificial Sequence Synthetic primer
ttaggtccaattattagtcaaggtaatga

SEQ ID NO: 37 - DNA, 26 bp, Artificial Sequence
  Description of Artificial Sequence Synthetic primer
atctgcattaaaagaaattggtggta

SEQ ID NO: 38 - DNA, 35 bp, Artificial Sequence
  Description of Artificial Sequence Synthetic probe
tcccaaaaatccactgttactgaactaggtttctc

SEQ ID NO: 39 - DNA, 28 bp, Artificial Sequence
  Description of Artificial Sequence Synthetic primer
catgtcaaaatgttctgaggtatattct

SEQ ID NO: 40 - DNA, 22 bp, Artificial Sequence
  Description of Artificial Sequence Synthetic primer
ttaacccagccatagagtctga

SEQ ID NO: 41 - DNA, 28 bp, Artificial Sequence
  Description of Artificial Sequence Synthetic probe
agcgagcttctggcataaaacctacttg

SEQ ID NO: 42 - DNA, 24 bp, Artificial Sequence
  Description of Artificial Sequence Synthetic primer
tcctggaccacttaaacttattgt

SEQ ID NO: 43 - DNA, 21 bp, Artificial Sequence
  Description of Artificial Sequence Synthetic primer
taacccagccatagagtctga

SEQ ID NO: 44 - DNA, 25 bp, Artificial Sequence
  Description of Artificial Sequence Synthetic primer
ctcctggaccacttaaacttattgt

SEQ ID NO: 45 - DNA, 20 bp, Artificial Sequence
  Description of Artificial Sequence Synthetic primer
aacccagccatagagtctga

SEQ ID NO: 46 - DNA, 28 bp, Artificial Sequence
  Description of Artificial Sequence Synthetic probe
tggagcgagcttctggcataaaacctac

SEQ ID NO: 47 - DNA, 24 bp, Artificial Sequence
  Description of Artificial Sequence Synthetic primer
ataagctcctggaccacttaaact

SEQ ID NO: 48 - DNA, 20 bp, Artificial Sequence
  Description of Artificial Sequence Synthetic primer
ccttaacccagccatagagt

SEQ ID NO: 49 - DNA, 27 bp, Artificial Sequence
  Description of Artificial Sequence Synthetic probe
agcgagcttctggcataaaacctactt

SEQ ID NO: 50 - DNA, 20 bp, Artificial Sequence
  Description of Artificial Sequence Synthetic primer
gctcctggaccacttaaact

SEQ ID NO: 51 - DNA, 20 bp, Artificial Sequence
  Description of Artificial Sequence Synthetic primer
caacaccttaacccagccat

SEQ ID NO: 52 - DNA, 23 bp, Artificial Sequence
  Description of Artificial Sequence Synthetic primer
ataagctcctggaccacttaaac

SEQ ID NO: 53 - DNA, 22 bp, Artificial Sequence
  Description of Artificial Sequence Synthetic primer
caacaccttaacccagccatag

SEQ ID NO: 54 - DNA, 21 bp, Artificial Sequence
  Description of Artificial Sequence Synthetic primer
agctcctggaccacttaaact

SEQ ID NO: 55 - DNA, 26 bp, Artificial Sequence
  Description of Artificial Sequence Synthetic primer
aatttttaaaccaacaccttaaccca

SEQ ID NO: 56 - DNA, 29 bp, Artificial Sequence
  Description of Artificial Sequence Synthetic probe
agcgagcttctggcataaaacctacttgt

SEQ ID NO: 57 - DNA, 23 bp, Artificial Sequence
  Description of Artificial Sequence Synthetic primer
taagctcctggaccacttaaact

SEQ ID NO: 58 - DNA, 30 bp, Artificial Sequence
  Description of Artificial Sequence Synthetic primer
ccatttatcccaaataacaatttctttgac

SEQ ID NO: 59 - DNA, 33 bp, Artificial Sequence
  Description of Artificial Sequence Synthetic probe
ccaaatcttcgtcttcccaatcagacatcaact

SEQ ID NO: 60 - DNA, 29 bp, Artificial Sequence
  Description of Artificial Sequence Synthetic primer
agtccttaatagtatatccatttcgttca

SEQ ID NO: 61 - DNA, 35 bp, Artificial Sequence
  Description of Artificial Sequence Synthetic probe
aaatcttcgtcttcccaatcagacatcaactttgg

SEQ ID NO: 62 - DNA, 30 bp, Artificial Sequence
  Description of Artificial Sequence Synthetic primer
aagtccttaatagtatatccatttcgttca

SEQ ID NO: 63 - DNA, 27 bp, Artificial Sequence
  Description of Artificial Sequence Synthetic primer
agacgaagatttggatacagataatga

SEQ ID NO: 64 - DNA, 35 bp, Artificial Sequence
  Description of Artificial Sequence Synthetic probe
ttcttatagccttgttctgcaaaactatcttccca

SEQ ID NO: 65 - DNA, 21 bp, Artificial Sequence
  Description of Artificial Sequence Synthetic primer
tggatctccagcagtatttga

SEQ ID NO: 66 - DNA, 28 bp, Artificial Sequence
  Description of Artificial Sequence Synthetic primer
aagacgaagatttggatacagataatga

SEQ ID NO: 67 - DNA, 23 bp, Artificial Sequence
  Description of Artificial Sequence Synthetic primer
tatggatctccagcagtatttga

SEQ ID NO: 68 - DNA, 30 bp, Artificial Sequence
  Description of Artificial Sequence Synthetic primer
gatgatccatttatcccaaataacaatttc

SEQ ID NO: 69 - DNA, 34 bp, Artificial Sequence
  Description of Artificial Sequence Synthetic probe
ccaaatcttcgtcttcccaatcagacatcaactt

SEQ ID NO: 70 - DNA, 24 bp, Artificial Sequence
  Description of Artificial Sequence Synthetic primer
cagaccgatagcatagcacttaaa

SEQ ID NO: 71 - DNA, 32 bp, Artificial Sequence
  Description of Artificial Sequence Synthetic probe
tgctgctctgacaactatactctcaggataca

SEQ ID NO: 72 - DNA, 20 bp, Artificial Sequence
  Description of Artificial Sequence Synthetic primer
tcccttggtggtgaatcaat

SEQ ID NO: 73 - DNA, 324 bp, Artificial Sequence
  Description of Artificial Sequence Synthetic target polynucleotide
gcgaagtgagaatacgccgtgtcgcagtttccttgagcagtgtctctaaatgcctcaaaccgtcgcatttttggttatagcagtaactatatggaggtccgtaggcggcgtgcgtgggggcaccaaactcatccaacggtcgactgcgcctgtagggtcttaagaagcggcacctcagaccgatagcatagcacttaaagaggaattgaataatcaagatgggtatccgaccgacgcggagtgaccgaggaagaggaccctgcatgtatcctgagagtatagttgtcagagcagcaattgattcaccaccaagggacttagtct

SEQ ID NO: 74 - DNA, 20 bp, Artificial Sequence
  Description of Artificial Sequence Synthetic primer
caaacgaattagggcctgtt

SEQ ID NO: 75 - DNA, 20 bp, Artificial Sequence
  Description of Artificial Sequence Synthetic primer
ctgtttcagcgtttaggcat

SEQ ID NO: 76 - DNA, 21 bp, Artificial Sequence
  Description of Artificial Sequence Synthetic primer
gaaacaggggacataagtgtg

SEQ ID NO: 77 - DNA, 30 bp, Artificial Sequence
  Description of Artificial Sequence Synthetic probe
aacacacttatgtcccctgtttcatctcat

SEQ ID NO: 78 - DNA, 32 bp, Artificial Sequence
  Description of Artificial Sequence Synthetic probe
tcctttcattacttaacacacttatgtcccct

SEQ ID NO: 79 - DNA, 23 bp, Artificial Sequence
  Description of Artificial Sequence Synthetic primer
cgaacctgttcctttcattactt

SEQ ID NO: 80 - DNA, 26 bp, Artificial Sequence
  Description of Artificial Sequence Synthetic primer
ccattatattttctcatcgaacctgt

SEQ ID NO: 81 - DNA, 27 bp, Artificial Sequence
  Description of Artificial Sequence Synthetic probe
cccgatacattgttccgtccaaatcaa

SEQ ID NO: 82 - DNA, 18 bp, Artificial Sequence
  Description of Artificial Sequence Synthetic primer
catcaatccgctccgttc

SEQ ID NO: 83 - DNA, 26 bp, Artificial Sequence
  Description of Artificial Sequence Synthetic primer
gaaaatgcagatgttgtaattgtagg

SEQ ID NO: 84 - DNA, 25 bp, Artificial Sequence
  Description of Artificial Sequence Synthetic primer
aaatgcagatgttgtaattgtaggt

SEQ ID NO: 85 - DNA, 24 bp, Artificial Sequence
  Description of Artificial Sequence Synthetic primer
atgcagatgttgtaattgtaggtc

SEQ ID NO: 86 - DNA, 23 bp, Artificial Sequence
  Description of Artificial Sequence Synthetic primer
tgcagatgttgtaattgtaggtc

SEQ ID NO: 87 - DNA, 24 bp, Artificial Sequence
  Description of Artificial Sequence Synthetic primer
taattgtaggtctagatcgggaag

SEQ ID NO: 88 - DNA, 22 bp, Artificial Sequence
  Description of Artificial Sequence Synthetic primer
attgtaggtctagatcgggaag

SEQ ID NO: 89 - DNA, 23 bp, Artificial Sequence
  Description of Artificial Sequence Synthetic primer
tctagatcgggaagttacgtatg

SEQ ID NO: 90 - DNA, 24 bp, Artificial Sequence
  Description of Artificial Sequence Synthetic primer
gatcgggaagttacgtatgaaaaa

SEQ ID NO: 91 - DNA, 26 bp, Artificial Sequence
  Description of Artificial Sequence Synthetic primer
aagttacgtatgaaaaattggctgta

SEQ ID NO: 92 - DNA, 24 bp, Artificial Sequence
  Description of Artificial Sequence Synthetic primer
agttacgtatgaaaaattggctgt

SEQ ID NO: 93 - DNA, 18 bp, Artificial Sequence
  Description of Artificial Sequence Synthetic primer
tgtagcctgtcttgctgt

SEQ ID NO: 94 - DNA, 30 bp, Artificial Sequence
  Description of Artificial Sequence Synthetic probe
caagacaggctacagccaatttttcatacg

SEQ ID NO: 95 - DNA, 28 bp, Artificial Sequence
  Description of Artificial Sequence Synthetic probe
acgaaatggtgccaaattgatctcaacg

SEQ ID NO: 96 - DNA, 20 bp, Artificial Sequence
  Description of Artificial Sequence Synthetic primer
tttggcaccatttcgtacag

SEQ ID NO: 97 - DNA, 29 bp, Artificial Sequence
  Description of Artificial Sequence Synthetic probe
tgagatcaatttggcaccatttcgtacag

SEQ ID NO: 98 - DNA, 30 bp, Artificial Sequence
  Description of Artificial Sequence Synthetic probe
ttgagatcaatttggcaccatttcgtacag

SEQ ID NO: 99 - DNA, 30 bp, Artificial Sequence
  Description of Artificial Sequence Synthetic probe
attcgttgagatcaatttggcaccatttcg

SEQ ID NO: 100 - DNA, 28 bp, Artificial Sequence
  Description of Artificial Sequence Synthetic probe
ctccattcgttgagatcaatttggcacc

SEQ ID NO: 101 - DNA, 28 bp, Artificial Sequence
  Description of Artificial Sequence Synthetic probe
tctccattcgttgagatcaatttggcac

SEQ ID NO: 102 - DNA, 29 bp, Artificial Sequence
  Description of Artificial Sequence Synthetic probe
atctccattcgttgagatcaatttggcac

SEQ ID NO: 103 - DNA, 21 bp, Artificial Sequence
  Description of Artificial Sequence Synthetic primer
taaggcaagatctccattcgt

SEQ ID NO: 104 - DNA, 21 bp, Artificial Sequence
  Description of Artificial Sequence Synthetic primer
gtaaggcaagatctccattcg

SEQ ID NO: 105 - DNA, 19 bp, Artificial Sequence
  Description of Artificial Sequence Synthetic primer
tcggtaaggcaagatctcc

SEQ ID NO: 106 - DNA, 19 bp, Artificial Sequence
  Description of Artificial Sequence Synthetic primer
ttcagtcggtaaggcaaga

SEQ ID NO: 107 - DNA, 20 bp, Artificial Sequence
  Description of Artificial Sequence Synthetic primer
ttcagtcggtaaggcaagat

SEQ ID NO: 108 - DNA, 20 bp, Artificial Sequence
  Description of Artificial Sequence Synthetic primer
ctttcagtcggtaaggcaag

SEQ ID NO: 109 - DNA, 19 bp, Artificial Sequence
  Description of Artificial Sequence Synthetic primer
ccggcataaatcccctttc

SEQ ID NO: 110 - DNA, 18 bp, Artificial Sequence
  Description of Artificial Sequence Synthetic primer
ggccgaaaaagaatcgga

SEQ ID NO: 111 - DNA, 18 bp, Artificial Sequence
  Description of Artificial Sequence Synthetic primer
tgggccgaaaaagaatcg

SEQ ID NO: 112 - DNA, 19 bp, Artificial Sequence
  Description of Artificial Sequence Synthetic primer
ttgggccgaaaaagaatcg

SEQ ID NO: 113 - DNA, 21 bp, Artificial Sequence
  Description of Artificial Sequence Synthetic primer
gaattgggccgaaaaagaatc

SEQ ID NO: 114 - DNA, 20 bp, Artificial Sequence
  Description of Artificial Sequence Synthetic primer
tacgaattgggccgaaaaag

SEQ ID NO: 115 - DNA, 19 bp, Artificial Sequence
  Description of Artificial Sequence Synthetic primer
ttacgaattgggccgaaaa

SEQ ID NO: 116 - DNA, 18 bp, Artificial Sequence
  Description of Artificial Sequence Synthetic primer
taagaagctcgccgttac

SEQ ID NO: 117 - DNA, 19 bp, Artificial Sequence
  Description of Artificial Sequence Synthetic primer
ttaagaagctcgccgttac

SEQ ID NO: 118 - DNA, 18 bp, Artificial Sequence
  Description of Artificial Sequence Synthetic primer
cttaagaagctcgccgtt

SEQ ID NO: 119 - DNA, 19 bp, Artificial Sequence
  Description of Artificial Sequence Synthetic primer
cttaagaagctcgccgtta

SEQ ID NO: 120 - DNA, 20 bp, Artificial Sequence
  Description of Artificial Sequence Synthetic primer
cttaagaagctcgccgttac

SEQ ID NO: 121 - DNA, 26 bp, Artificial Sequence
  Description of Artificial Sequence Synthetic probe
cttaagaagctcgccgttacgaattg

SEQ ID NO: 122 - DNA, 20 bp, Artificial Sequence
  Description of Artificial Sequence Synthetic primer
acttaagaagctcgccgtta

SEQ ID NO: 123 - DNA, 28 bp, Artificial Sequence
  Description of Artificial Sequence Synthetic probe
cacttaagaagctcgccgttacgaattg

SEQ ID NO: 124 - DNA, 27 bp, Artificial Sequence
  Description of Artificial Sequence Synthetic probe
ccacttaagaagctcgccgttacgaat

SEQ ID NO: 125 - DNA, 19 bp, Artificial Sequence
  Description of Artificial Sequence Synthetic primer
caagagcctttcttgtcca

SEQ ID NO: 126 - DNA, 18 bp, Artificial Sequence
  Description of Artificial Sequence Synthetic primer
ggcaagagcctttcttgt

SEQ ID NO: 127 - DNA, 18 bp, Artificial Sequence
  Description of Artificial Sequence Synthetic primer
gccaaattgtgcaatcgt

SEQ ID NO: 128 - DNA, 19 bp, Artificial Sequence
  Description of Artificial Sequence Synthetic primer
gccaaattgtgcaatcgtt

SEQ ID NO: 129 - DNA, 28 bp, Artificial Sequence
  Description of Artificial Sequence Synthetic probe
tttcacaacctgagaatgtcacaacaca

SEQ ID NO: 130 - DNA, 29 bp, Artificial Sequence
  Description of Artificial Sequence Synthetic probe
tttcacaacctgagaatgtcacaacacat

SEQ ID NO: 131 - DNA, 21 bp, Artificial Sequence
  Description of Artificial Sequence Synthetic primer
ttcacaacctgagaatgtcac

SEQ ID NO: 132 - DNA, 28 bp, Artificial Sequence
  Description of Artificial Sequence Synthetic probe
ttcacaacctgagaatgtcacaacacat

SEQ ID NO: 133 - DNA, 30 bp, Artificial Sequence
  Description of Artificial Sequence Synthetic probe
ttcacaacctgagaatgtcacaacacatta

SEQ ID NO: 134 - DNA, 29 bp, Artificial Sequence
  Description of Artificial Sequence Synthetic probe
tcacaacctgagaatgtcacaacacatta

SEQ ID NO: 135 - DNA, 21 bp, Artificial Sequence
  Description of Artificial Sequence Synthetic primer
caacctgagaatgtcacaaca

SEQ ID NO: 136 - DNA, 21 bp, Artificial Sequence
  Description of Artificial Sequence Synthetic primer
aacctgagaatgtcacaacac

SEQ ID NO: 137 - DNA, 23 bp, Artificial Sequence
  Description of Artificial Sequence Synthetic primer
cctgagaatgtcacaacacatta

SEQ ID NO: 138 - DNA, 26 bp, Artificial Sequence
  Description of Artificial Sequence Synthetic primer
agtccttgatttataatacctttact

SEQ ID NO: 139 - DNA, 10 bp, Artificial Sequence
  Description of Artificial Sequence Synthetic probe
aaacacgtgc

SEQ ID NO: 140 - DNA, 10 bp, Artificial Sequence
  Description of Artificial Sequence Synthetic probe
ccttgttcca

SEQ ID NO: 141 - DNA, 10 bp, Artificial Sequence
  Description of Artificial Sequence Synthetic probe
cagggacgat

(19) United States
(12) Patent Application Publication
(10) Pub. No.: US 2011/0256535 A1
(43) Pub. Date: Oct. 20, 2011

(54) OPTIMIZED OLIGONUCLEOTIDES AND METHODS OF USING SAME FOR THE DETECTION, ISOLATION, AMPLIFICATION, QUANTIFICATION, MONITORING, SCREENING AND SEQUENCING OF CLOSTRIDIUM DIFFICILE GENES ENCODING TOXIN B, AND/OR TOXIN A AND/OR BINARY TOXIN

(75) Inventors: David L. DOLINGER, Medway, MA (US); James R. HULLY, Mundelein, IL (US); Alice A. JACOBS, Cambridge, MA (US); Chesley LESLIN, Boston, MA (US); Heinz R. REISKE, Framingham, MA (US); Chunyang ZHENG, Tianjin (CN)

(73) Assignee: Intelligent Medical Devices, Inc.

(21) Appl. No.: 13/024,896

(22) Filed: Feb. 10, 2011

Related U.S. Application Data
(60) Provisional application No. 61/303,494, filed on Feb. 11, 2010.

(51) Int. Cl. C12Q 1/68; C07H 1/06; G01N 33/53; C07H 21/04; C12P 19/34

(52) U.S. Cl. 435/6.11; 536/24.33; 536/24.32; 435/91.5; 435/6.12; 436/501; 536/25.4

(57) ABSTRACT

Described herein are oligonucleotides useful for detecting, isolating, amplifying, quantitating, monitoring, screening and sequencing the C. Difficile genes encoding toxin B, and/or toxin A, and/or binary toxin, and methods of using the described oligonucleotides.

### RELATED APPLICATION

This application claims the benefit of U.S. Provisional Application No. 61/303,494, filed on Feb. 11, 2010, the contents of which are incorporated by reference herein in their entirety.

### FIELD OF THE INVENTION

The present invention relates to oligonucleotides for detecting Clostridium difficile, including methods for using these oligonucleotides for the detection, isolation, amplification, quantification, monitoring, screening and sequencing of Clostridium difficile genes encoding toxin B, and/or toxin A, and/or binary toxin.

### BACKGROUND

Clostridium difficile (C. Difficile) is a spore-forming, anaerobic, gram-positive bacillus that is recognized as the main etiological agent of antibiotic-associated diarrhea and pseudomembranous colitis. The use of antibiotics disrupts the normal intestinal flora, predisposing patients to colonization by C. Difficile. This is a disease which is encountered mainly in health care centers. The high level of healthy carriers among hospitalized patients, coupled with the presence of patients receiving antibiotic treatment, are some reasons for the high rate of nosocomial diarrhea associated with C. Difficile.

C. Difficile also has been observed as an etiological agent of appendicitis as well as diseases in other organs. C. Difficile can cause pseudomembranous enteritis (small bowel infection), osteomyelitis (bone infection), cellulitis (skin infection) and necrotizing fasciitis (soft tissue infection) as well as infection of prosthetic devices. C. Difficile may also cause reactive arthritis, most commonly in the knees and wrists.

C. Difficile infection (CDI) is considered one of the most important health care-associated infections. The main routes of transmission that cause the spread of bacteria among hospitalized patients are fecal-oral route or aerosols. Infected persons with acute diarrhea can excrete 10⁷ to 10⁹ micro-organisms per gram of feces leading to heavy contamination of the environment with spores. A patient can be exposed to C. Difficile spores through contact with the hospital environment or health care workers. After taking an antibiotic, the patient develops CDI if he or she acquires a toxigenic C. Difficile strain and fails to mount an anamnestic response to the bacteria's toxin. If the patient can mount an antibody response, he or she becomes asymptomatically colonized with C. Difficile. If the patient acquires a non-toxigenic C. Difficile strain, the patient also becomes asymptomatically colonized. Colonized patients have been shown to be protected from CDI.

It is estimated that there are approximately 500,000 cases of CDI per year in US hospitals and long-term care facilities (hospital-acquired CDI), and an estimated 15,000 to 20,000 patients die from CDI in the United States each year. Community-associated CDI, without previous direct or indirect contact with a hospital environment, remains rare compared with hospital-acquired CDI.

The most common symptoms of mild to moderate C. Difficile disease are watery diarrhea three or more times a day for two or more days and mild abdominal pain and tenderness. In more severe cases, C. Difficile causes the colon to become inflamed (colitis) or to form patches of raw tissue that can bleed or produce pus. Signs and symptoms of severe infection include watery diarrhea 10 to 15 times a day, abdominal pain which may be severe, fever, blood or pus in the stool, nausea, dehydration, loss of appetite, and weight loss. The standard treatment for C. Difficile infection is oral vancomycin or intravenous metronidazole.

Infection control measures to prevent CDI in hospitals are of two main types: those that attempt to prevent C. Difficile spores from reaching patients and those that reduce the risk of CDI should the patient ingest the organism. Isolation of patients with CDI and the use of gowns and gloves by health care workers are effective barrier methods. Hand washing is also another important barrier method. In addition, a sporicidal hypochlorite solution can significantly reduce spore contamination and CDI rates.

C. Difficile is difficult to culture as it takes 2 to 3 days to grow on 5% sheep's blood supplemented agar plates under anaerobic conditions at 37° C. The traditional gold standard for C. Difficile diagnosis is a cytotoxin assay that detects the cell cytotoxicity of toxin B and/or A (depending on the cell line used) in fecal eluate. Either toxin A and/or toxin B is confirmed as the cause by neutralization of the cytotoxic effect using specific anti-toxin antibodies. An alternative reference standard test is to culture C. Difficile by a method referred to as cytotoxigenic culture, which detects C. Difficile strains that have the capacity to produce toxin (or toxins) as opposed to detecting the presence of toxins in a stool sample. Several toxin detection kits are commercially available, however, the positive predictive value (PPV) of these assays is unacceptably low (<50% in some cases).

There are currently several real-time PCR assays for C. Difficile in the market. When compared to culture, the PCR assays are faster (hours versus days) and exceed the analytical sensitivity of a culture-based method. When compared to immunoassays, the real-time PCR assays are more sensitive and specific. A positive result in a real-time PCR assay may suggest the presence of a C. Difficile toxin gene (such as toxin B) but does not necessarily mean that the toxin is being expressed. Therefore, the real-time PCR assay will be able to detect a C. Difficile strain that carries the gene for a toxin but is not expressing the toxin protein.

There is a need for rapid and accurate qualitative and quantitative real-time PCR reagents for the detection of toxin A (tcdA), toxin B (tcdB), and binary toxin genes, with robust precision and sensitivity. Specifically, there is a need for qualitative and quantitative real-time PCR reagents that can be used in a multiplex format for detection of each of the C. Difficile toxins. A rapid and accurate diagnostic test for the detection of various C. Difficile strains based on the genes for certain toxins, e.g., toxin A, toxin B, binary toxin, therefore, would provide clinicians with an effective tool for identifying patients or persons that are carriers of C. Difficile or identify C. Difficile as the cause of a specific disease or syndrome.

### SUMMARY

Described herein are oligonucleotides for detecting, isolating, amplifying, quantitating, screening and sequencing bacterial genetic material from the species C. Difficile, including detecting the tcdB gene, tcdA gene, and the binary toxin gene and methods for the use of these oligonucleotides. A diagnostic test that can detect C. Difficile strains based on toxin genes (tcdB, tcdA, and cdtB) is necessary because this pathogen is considered one of the worst health care-associated infections. Furthermore, a screening test is critical to enable the quick and informative determination of whether or not an individual is colonized with C. Difficile at the point of admission, or throughout an individual's stay, in a hospital and/or medical care setting.

One embodiment is directed to an isolated nucleic acid sequence comprising a sequence selected from the group consisting of: SEQ ID NOS 1-69 and 138.

One embodiment is directed to a method of hybridizing one or more isolated nucleic acid sequences comprising a sequence selected from the group consisting of: SEQ ID NOS: 1-69 and 138 to a C. Difficile sequence, comprising contacting one or more isolated nucleic acid sequences to a sample comprising the C. Difficile sequence under conditions suitable for hybridization. In a particular embodiment, the C. Difficile sequence is a genomic sequence, a template sequence or a sequence derived from an artificial construct. In a particular embodiment, the method(s) further comprise isolating, amplifying, quantitating, monitoring and/or sequencing the hybridized C. Difficile sequence.

One embodiment is directed to a primer set comprising at least one forward primer selected from the group consisting of SEQ ID NOS: 1, 4, 6, 8, 10, 12, 13, 18, 21, 23, 24, 26, 28, 30, 35, 36, 37, 40, 43, 45, 48, 51, 53, 55, 58, 63, 66, and 68, and at least one reverse primer selected from the group consisting of SEQ ID NOS: 3, 5, 7, 9, 11, 15, 17, 20, 25, 32, 33, 34, 39, 42, 44, 47, 50, 52, 54, 57, 60, 62, 65, 67 and 138. In a particular embodiment, the primer set is selected from the group consisting of: Groups 1-129 and 184 of Table 4, Groups 130-138 of Table 5, and Groups 139-145 of Table 6.

One embodiment is directed to a method of producing a nucleic acid product, comprising contacting one or more isolated nucleic acid sequences selected from the group consisting of SEQ ID NOS: 1, 3, 4, 5, 6, 7, 8, 9, 10, 11, 12, 13, 15, 17, 18, 20, 21, 23, 24, 25, 26, 28, 30, 32, 33, 34, 35, 36, 37, 39, 40, 42, 43, 44, 45, 47, 48, 50, 51, 52, 53, 54, 55, 57, 58, 60, 62, 63, 65, 66, 67, 68 and 138 to a sample comprising a C. Difficile sequence under conditions suitable for nucleic acid polymerization. In a particular embodiment, the nucleic acid product is an amplicon produced using at least one forward primer selected from the group consisting of SEQ ID NOS: 1, 4, 6, 8, 10, 12, 13, 18, 21, 23, 24, 26, 28, 30, 35, 36, 37, 40, 43, 45, 48, 51, 53, 55, 58, 63, 66, and 68, and at least one reverse primer selected from the group consisting of SEQ ID NOS: 3, 5, 7, 9, 11, 15, 17, 20, 25, 32, 33, 34, 39, 42, 44, 47, 50, 52, 54, 57, 60, 62, 65, 67 and 138.

Particular embodiments are directed to primers and probes that hybridize to, amplify and/or detect C. Difficile toxins selected from the group consisting of: tcdB, tcdA, and cdtB, and methods of using the primers and probes.

One embodiment is directed to a probe that hybridizes to an amplicon produced as described herein, e.g., using the primers described herein. In a particular embodiment, the probe comprises a sequence selected from the group consisting of: SEQ ID NOS: 2, 14, 16, 19, 22, 27, 29, 31, 38, 41, 46, 49, 56, 59, 61, 64, and 69. In a particular embodiment, the probe is labeled with a detectable label selected from the group consisting of: a fluorescent label, a chemiluminescent label, a quencher, a radioactive label, biotin, mass tags and/or gold. The probe may also be labeled with other similar detectable labels used in conjunction with probe technology as known by one of ordinary skill in the art.

One embodiment is directed to a set of probes that hybridize to an amplicon produced as described herein, e.g., using the primers described herein. In a particular embodiment, a first probe comprises a sequence selected from the group consisting of: SEQ ID NOS: 2, 14, 16, 19, 22, 27, 29, 31, and 38, and a second probe comprises a sequence selected from the group consisting of: SEQ ID NOS: 41, 46, 49, and 56. In a particular embodiment, a first probe comprises a sequence selected from the group consisting of: SEQ ID NOS: 2, 14, 16, 19, 22, 27, 29, 31, and 38, a second probe comprises a sequence selected from the group consisting of: SEQ ID NOS: 41, 46, 49, and 56, and a third probe comprises a sequence selected from the group consisting of: SEQ ID NOS: 59, 61, 64, and 69. In a particular embodiment, the first probe is labeled with a first detectable label and the second probe is labeled with a second detectable label. In a particular embodiment, the first probe and the second probe are labeled with the same detectable label. In a particular embodiment, the first probe is labeled with a first detectable label, the second probe is labeled with a second detectable label and the third probe is labeled with a third detectable label. In a particular embodiment, the first probe, the second probe and the third probe are labeled with the same detectable label. In a particular embodiment, the first probe and the third probe are labeled with a first detectable label and the second probe is labeled with a second detectable label. In a particular embodiment, the first probe is labeled with a first detectable label and the second probe and third probe are labeled with a second detectable label. In a particular embodiment, the detectable labels are selected from the group consisting of: a fluorescent label, a chemiluminescent label, a quencher, a radioactive label, biotin, mass tags and gold. The probe may also be labeled with other similar detectable labels used in conjunction with probe technology as known by one of ordinary skill in the art.

One embodiment is directed to a method for detecting a C. Difficile sequence in a sample, comprising: a) contacting the sample with at least one forward primer comprising a sequence selected from the group consisting of: SEQ ID NOS: 1, 4, 6, 8, 10, 12, 13, 18, 21, 23, 24, 26, 28, 30, 35, 36, 37, 40, 43, 45, 48, 51, 53, 55, 58, 63, 66, and 68, and at least one reverse primer comprising a sequence selected from the group consisting of: SEQ ID NOS: 3, 5, 7, 9, 11, 15, 17, 20, 25, 32, 33, 34, 39, 42, 44, 47, 50, 52, 54, 57, 60, 62, 65, 67 and 138 under conditions such that nucleic acid amplification occurs to yield an amplicon; and b) contacting the amplicon with one or more probes comprising one or more sequences selected from the group consisting of: SEQ ID NOS: 2, 14, 16, 19, 22, 27, 29, 31, 38, 41, 46, 49, 56, 59, 61, 64, and 69 under conditions such that hybridization of the probe to the amplicon occurs, wherein hybridization of the probe is indicative of C. Difficile in the sample. In a particular embodiment, each of the one or more probes is labeled with a different detectable label. In a particular embodiment, the one or more probes are labeled with the same detectable label. In a particular embodiment, the sample is selected from the group consisting of: blood, serum, plasma, enriched peripheral blood mononuclear cells, fecal material, urine, neoplastic or other tissue obtained from biopsies, cerebrospinal fluid, saliva, fluids collected from the ear, eye, mouth, and respiratory airways, sputum, stool, skin, gastric secretions, oropharyngeal swabs, nasopharyngeal swabs, throat swabs, rectal swabs, nasal aspirates, nasal wash, renal tissue, and fluid therefrom including perfusion media, pure cultures of bacterial fungal isolates, fluids and cells obtained by the perfusion of tissues of both human and animal origin, and fluids and cells derived from the culturing of human cells, including human stem cells and human cartilage or fibroblasts, pure cultures of bacterial fungal isolates, and swabs or washes of environmental surfaces, or other samples derived from environmental surfaces. In a particular embodiment, the sample is from a human, is non-human in origin, or is derived from an inanimate object.

In a particular embodiment, the at least one forward primer, the at least one reverse primer and the one or more probes are selected from the group consisting of: Groups 1-129 and 184 of Table 4, Groups 130-138 of Table 5, and Groups 139-145 of Table 6. In a particular embodiment, the method(s) further comprise quantitating and/or sequencing C. Difficile sequences in a sample.

One embodiment is directed to a primer set or collection of primer sets for amplifying sequences from C. Difficile, including the toxin genes tcdB, tcdA, and cdtB, comprising a nucleotide sequence selected from the group consisting of: (1) SEQ ID NOS: 1 and 3; (2) SEQ ID NOS: 13 and 15; (3) SEQ ID NOS: 13 and 17; (4) SEQ ID NOS: 18 and 20; (5) SEQ ID NOS: 21 and 15; (6) SEQ ID NOS: 23 and 20; (7) SEQ ID NOS: 24 and 25; (8) SEQ ID NOS: 26 and 15; (9) SEQ ID NOS: 28 and 20; (10) SEQ ID NOS: 4 and 5; (11) SEQ ID NOS: 6 and 7; (12) SEQ ID NOS: 8 and 9; (13) SEQ ID NOS: 10 and 11; (14) SEQ ID NOS: 12 and 5; (15) SEQ ID NOS: 30 and 32; (16) SEQ ID NOS: 37 and 39; (17) SEQ ID NOS: 30 and 33; (18) SEQ ID NOS: 30 and 34; (19) SEQ ID NOS: 35 and 32; (20) SEQ ID NOS: 35 and 33; (21) SEQ ID NOS: 35 and 34; (22) SEQ ID NOS: 36 and 32; (23) SEQ ID NOS: 36 and 33; (24) SEQ ID NOS: 36 and 34; (25) SEQ ID NOS: 40 and 42; (26) SEQ ID NOS: 43 and 44; (27) SEQ ID NOS: 45 and 47; (28) SEQ ID NOS: 48 and 50; (29) SEQ ID NOS: 51 and 42; (30) SEQ ID NOS: 48 and 52; (31) SEQ ID NOS: 53 and 54; (32) SEQ ID NOS: 55 and 42; (33) SEQ ID NOS: 55 and 57; (34) SEQ ID NOS: 58 and 60; (35) SEQ ID NOS: 58 and 62; (36) SEQ ID NOS: 63 and 65; (37) SEQ ID NOS: 66 and 67; (38) SEQ ID NOS: 68 and 60 and (39) SEQ ID NOS: 28 and 138. A particular embodiment is directed to oligonucleotide probes for binding to the C. Difficile sequences encoding toxin B gene, toxin A gene, and binary toxin gene comprising a nucleotide sequence selected from the group consisting of: SEQ ID NOS: 2, 14, 16, 19, 22, 27, 29, 31, 38, 41, 46, 49, 56, 59, 61, 64, and 69.

One embodiment is directed to a primer set for amplifying sequences from a C. Difficile toxin B gene, comprising a nucleotide sequence selected from the group consisting of: (1) SEQ ID NOS: 1 and 3; (2) SEQ ID NOS: 13 and 15; (3) SEQ ID NOS: 13 and 17; (4) SEQ ID NOS: 18 and 20; (5) SEQ ID NOS: 21 and 15; (6) SEQ ID NOS: 23 and 20; (7) SEQ ID NOS: 24 and 25; (8) SEQ ID NOS: 26 and 15; (9) SEQ ID NOS: 28 and 20; (10) SEQ ID NOS: 4 and 5; (11) SEQ ID NOS: 6 and 7; (12) SEQ ID NOS: 8 and 9; (13) SEQ ID NOS: 10 and 11; (14) SEQ ID NOS: 12 and 5; (15) SEQ ID NOS: 30 and 32; (16) SEQ ID NOS: 37 and 39; (17) SEQ ID NOS: 30 and 33; (18) SEQ ID NOS: 30 and 34; (19) SEQ ID NOS: 35 and 32; (20) SEQ ID NOS: 35 and 33; (21) SEQ ID NOS: 35 and 34; (22) SEQ ID NOS: 36 and 32; (23) SEQ ID NOS: 36 and 33; (24) SEQ ID NOS: 36 and 34 and (25) SEQ ID NOS: 28 and 138. A particular embodiment is directed to oligonucleotide probes for binding to sequences encoding the C. Difficile toxin B gene, comprising a nucleotide sequence selected from the group consisting of: SEQ ID NOS: 2, 14, 16, 19, 22, 27, 29, 31, and 38.

One embodiment is directed to a primer set for amplifying sequences from a C. Difficile toxin A gene, comprising a nucleotide sequence selected from the group consisting of: (1) SEQ ID NOS: 40 and 42; (2) SEQ ID NOS: 43 and 44; (3) SEQ ID NOS: 45 and 47; (4) SEQ ID NOS: 48 and 50; (5) SEQ ID NOS: 51 and 42; (6) SEQ ID NOS: 48 and 52; (7) SEQ ID NOS: 53 and 54; (8) SEQ ID NOS: 55 and 42; and (9) SEQ ID NOS: 55 and 57. A particular embodiment is directed to oligonucleotide probes for binding to the C. Difficile toxin A gene, comprising a nucleotide sequence selected from the group consisting of: SEQ ID NOS: 41, 46, 49, and 56.

One embodiment is directed to a primer set for amplifying sequences from a C. Difficile binary toxin gene, comprising a nucleotide sequence selected from the group consisting of: (1) SEQ ID NOS: 58 and 60; (2) SEQ ID NOS: 58 and 62; (3) SEQ ID NOS: 63 and 65; (4) SEQ ID NOS: 66 and 67; and (5) SEQ ID NOS: 68 and 60. A particular embodiment is directed to oligonucleotide probes for binding to the C. Difficile binary toxin gene, comprising a nucleotide sequence selected from the group consisting of: SEQ ID NOS: 59, 61, 64, and 69.

In one embodiment, the present invention is directed to simultaneous detection in a multiplex format of (1) tcdB (toxin B); and/or (2) tcdA (toxin A) and/or (3) cdtB (binary toxin). These probes will provide identification of C. Difficile containing genes that code for toxin B, and/or toxin A, and/or binary toxin. Such an embodiment can be used in a diagnostic assay or in a screening assay.

One embodiment is directed to primer sets for amplifying sequences from C. Difficile containing the genes for toxin B, and/or toxin A, and/or binary toxin, comprising
(a) (1) SEQ ID NOS: 1 and 3; (2) SEQ ID NOS: 13 and 15; (3) SEQ ID NOS: 13 and 17; (4) SEQ ID NOS: 18 and 20; (5) SEQ ID NOS: 21 and 15; (6) SEQ ID NOS: 23 and 20; (7) SEQ ID NOS: 24 and 25; (8) SEQ ID NOS: 26 and 15; (9) SEQ ID NOS: 28 and 20; (10) SEQ ID NOS: 4 and 5; (11) SEQ ID NOS: 6 and 7; (12) SEQ ID NOS: 8 and 9; (13) SEQ ID NOS: 10 and 11; (14) SEQ ID NOS: 12 and 5; (15) SEQ ID NOS: 30 and 32; (16) SEQ ID NOS: 37 and 39; (17) SEQ ID NOS: 30 and 33; (18) SEQ ID NOS: 30 and 34; (19) SEQ ID NOS: 35 and 32; (20) SEQ ID NOS: 35 and 33; (21) SEQ ID NOS: 35 and 34; (22) SEQ ID NOS: 36 and 32; (23) SEQ ID NOS: 36 and 33; (24) SEQ ID NOS: 36 and 34 and (25) SEQ ID NOS: 28 and 138 (forward and reverse primers for amplifying the tcdB gene); and
(b) (1) SEQ ID NOS: 40 and 42; (2) SEQ ID NOS: 43 and 44; (3) SEQ ID NOS: 45 and 47; (4) SEQ ID NOS: 48 and 50; (5) SEQ ID NOS: 51 and 42; (6) SEQ ID NOS: 48 and 52; (7) SEQ ID NOS: 53 and 54; (8) SEQ ID NOS: 55 and 42; and (9) SEQ ID NOS: 55 and 57 (forward and reverse primers for amplifying the tcdA gene); and
(c) (1) SEQ ID NOS: 58 and 60; (2) SEQ ID NOS: 58 and 62; (3) SEQ ID NOS: 63 and 65; (4) SEQ ID NOS: 66 and 67; and (5) SEQ ID NOS: 68 and 60 (forward and reverse primers for amplifying the cdtB gene). A particular embodiment is directed to oligonucleotide probes for binding to the toxin B, and/or toxin A, and/or binary toxin gene, comprising a nucleotide sequence selected from the group consisting of: SEQ ID NOS: 2, 14, 16, 19, 22, 27, 29, 31, and 38 (toxin B probe), SEQ ID NOS: 41, 46, 49, and 56 (toxin A probe) and SEQ ID NOS: 59, 61, 64, and 69 (binary toxin probes).

One embodiment is directed to a kit for detecting C. Difficile sequences in a sample, comprising one or more probes comprising a sequence selected from the group consisting of: SEQ ID NOS: 2, 14, 16, 19, 22, 27, 29, 31, 38, 41, 46, 49, 56, 59, 61, 64, and 69. In a particular embodiment, the kit further comprises a) at least one forward primer comprising the sequence selected from the group consisting of: SEQ ID NOS: 1, 4, 6, 8, 10, 12, 13, 18, 21, 23, 24, 26, 28, 30, 35, 36, 37, 40, 43, 45, 48, 51, 53, 55, 58, 63, 66, and 68; and b) at least one reverse primer comprising the sequence selected from the group consisting of: SEQ ID NOS: 3, 5, 7, 9, 11, 15, 17, 20, 25, 32, 33, 34, 39, 42, 44, 47, 50, 52, 54, 57, 60, 62, 65, 67 and 138. In a particular embodiment, the kit further comprises reagents for quantitating and/or sequencing C. Difficile sequences in the sample. In a particular embodiment, the one or more probes are labeled with different detectable labels. In a particular embodiment, the one or more probes are labeled with the same detectable label. In a particular embodiment, the at least one forward primer and the at least one reverse primer are selected from the group consisting of: Groups 1-129 and 184 of Table 4, Groups 130-138 of Table 5, and Groups 139-145 of Table 6.

One embodiment is directed to a method of diagnosing a C. Difficile-associated colonization, condition, syndrome or disease, comprising: a) contacting a sample with at least one forward and reverse primer set selected from the group consisting of: Groups 1-129 and 184 of Table 4, Groups 130-138 of Table 5, and Groups 139-145 of Table 6; b) conducting an amplification reaction, thereby producing an amplicon; and c) detecting the amplicon using one or more probes selected from the group consisting of: SEQ ID NOS: 2, 14, 16, 19, 22, 27, 29, 31, 38, 41, 46, 49, 56, 59, 61, 64, and 69; wherein the detection of an amplicon is indicative of the presence of C. Difficile in the sample. In a particular embodiment, the sample is selected from the group consisting of: blood, serum, plasma, enriched peripheral blood mononuclear cells, fecal material, urine, neoplastic or other tissue obtained from biopsies, cerebrospinal fluid, saliva, fluids collected from the ear, eye, mouth, and respiratory airways, sputum, stool, skin, gastric secretions, oropharyngeal swabs, nasopharyngeal swabs, throat swabs, rectal swabs, nasal aspirates, nasal wash, renal tissue, and fluid therefrom including perfusion media, pure cultures of bacterial fungal isolates, fluids and cells obtained by the perfusion of tissues of both human and animal origin, and fluids and cells derived from the culturing of human cells, including human stem cells and human cartilage or fibroblasts, pure cultures of bacterial fungal isolates, and swabs or washes of environmental surfaces, or other samples derived from environmental surfaces. In a particular embodiment, the sample is from a human, is non-human in origin, or is derived from an inanimate object. In a particular embodiment, the C. Difficile-associated colonization, condition, syndrome or disease is selected from the group consisting of: watery diarrhea, abdominal pain, inflamed colon (colitis), appendicitis, small bowel enteritis, reactive arthritis, cellulitis, necrotizing fasciitis, osteomyelitis, fever, blood or pus in the stool, nausea, dehydration, loss of appetite, and weight loss.

One embodiment is directed to a kit for amplifying and sequencing C. Difficile sequences in a sample, comprising: a) at least one forward primer comprising the sequence selected from the group consisting of: SEQ ID NOS: 1, 4, 6, 8, 10, 12, 13, 18, 21, 23, 24, 26, 28, 30, 35, 36, 37, 40, 43, 45, 48, 51, 53, 55, 58, 63, 66, and 68; b) at least one reverse primer comprising the sequence selected from the group consisting of: SEQ ID NOS: 3, 5, 7, 9, 11, 15, 17, 20, 25, 32, 33, 34, 39, 42, 44, 47, 50, 52, 54, 57, 60, 62, 65, 67 and 138; c) reagents for the sequencing of amplified DNA fragments; and d) an internal control, positive control plasmids or a process control. In a particular embodiment, the kit further comprises reagents for quantitating C. Difficile sequences in the sample.

One embodiment is directed to an internal control plasmid and positive control plasmids.

The non-competitive internal control plasmid is a synthetic target that does not occur naturally in clinical sample types for which this assay is intended. The synthetic target sequence incorporates an artificial, random polynucleotide sequence with a known GC content. The synthetic target sequence is:

(SEQ ID NO: 73)
5′-GCGAAGTGAGAATACGCCGTGTCGCAGTTTCCTTGAGCAGTGTCT
CTAAATGCCTCAAACCGTCGCATTTTTGGTTATAGCAGTAACTATATG
GAGGTCCGTAGGCGGCGTGCGTGGGGGCACCAAACTCATCCAACGGTC
GACTGCGCCTGTAGGGTCTTAAGAAGCGGCACCTCAGACCGATAGCAT
AGCACTTAAAGAGGAATTGAATAATCAAGATGGGTATCCGACCGACGC
GGAGTGACCGAGGAAGAGGACCCTGCATGTATCCTGAGAGTATAGTTG
TCAGAGCAGCAATTGATTCACCACCAAGGGACTTAGTCT-3′.

This internal control is detected by a forward primer (SEQ ID NO: 70), a reverse primer (SEQ ID NO: 72) and a probe (SEQ ID NO: 71). A plasmid vector containing the internal control target sequence (SEQ ID NO: 73) is included in the assay. The internal control plasmid is added directly to the reaction mix to monitor the integrity of the PCR reagents and the presence of PCR inhibitors.

The C. Difficile positive control plasmid contains partial sequences for one or more of the C. Difficile targets (i.e., toxin A and/or toxin B and/or binary toxin). The positive control plasmid comprises forward primer, probe and reverse primer sequences for the given C. Difficile targets. An artificial polynucleotide sequence is inserted within the positive control sequence corresponding to the given target to allow the amplicon generated by the target primer pairs to be differentiated from the amplicon derived by the same primer pairs from a natural target by size, by a unique restriction digest profile, and by a probe directed against the artificial sequence. The positive control plasmids are intended to be used as a control to confirm that the assay is performing within specifications.

Another embodiment of the invention is directed to a process control. Bacterial material from an organism not related to Clostridium is incorporated into a kit (referred to hereinafter as the “process control bacterial material”). The process control bacterial material will be cultured and aliquoted at a known titer. These aliquots will be provided as nucleic acid extraction controls. Known amounts of the process control bacterial material will be spiked into a test sample by the user of the test kit. Nucleic acids will be extracted from the test sample and subjected to PCR to detect C. Difficile and the process control bacterial nucleic acids. Detection of the process control bacterial nucleic acids indicates that nucleic acid extraction from the test sample was successful.

One embodiment is directed to a method of diagnosing a C. Difficile-associated colonization, condition, syndrome or disease, comprising contacting a denatured target from a sample with one or more probes comprising a sequence selected from the group consisting of: SEQ ID NOS: 2, 14, 16, 19, 22, 27, 29, 31, 38, 41, 46, 49, 56, 59, 61, 64, and 69 under conditions for hybridization to occur; wherein hybridization of the one or more probes to a denatured target is indicative of the presence of C. Difficile in the sample. In a particular embodiment, the sample is selected from the group consisting of: blood, serum, plasma, enriched peripheral blood mononuclear cells, urine, neoplastic or other tissue obtained from biopsies, cerebrospinal fluid, saliva, fluids collected from the ear, eye, mouth, and respiratory airways, sputum, stool, fecal material, skin, gastric secretions, oropharyngeal swabs, nasopharyngeal swabs, throat swabs, rectal swabs, nasal aspirates, nasal wash, renal tissue, and fluid therefrom including perfusion media, pure cultures of bacterial fungal isolates, fluids and cells obtained by the perfusion of tissues of both human and animal origin, and fluids and cells derived from the culturing of human cells, including human stem cells and human cartilage or fibroblasts, pure cultures of bacterial fungal isolates, and swabs or washes of environmental surfaces, or other samples derived from environmental surfaces. In a particular embodiment, the sample is from a human, is non-human in origin, or is derived from an inanimate object. In a particular embodiment, the C. Difficile-associated colonization, condition, syndrome or disease is selected from the group consisting of: watery diarrhea, abdominal pain, inflamed colon (colitis), appendicitis, small bowel enteritis, reactive arthritis, cellulitis, necrotizing fasciitis, osteomyelitis, fever, blood or pus in the stool, nausea, dehydration, loss of appetite, and weight loss.

One embodiment is directed to a method for identifying the causative agent of watery diarrhea by detecting one or more of the toxin genes of a C. Difficile species in a sample, the method comprising: a) contacting the sample with at least one forward primer comprising the sequence selected from the group consisting of: SEQ ID NOS: 1, 4, 6, 8, 10, 12, 13, 18, 21, 23, 24, 26, 28, 30, 35, 36, 37, 40, 43, 45, 48, 51, 53, 55, 58, 63, 66, and 68 and at least one reverse primer comprising the sequence selected from the group consisting of: SEQ ID NOS: 3, 5, 7, 9, 11, 15, 17, 20, 25, 32, 33, 34, 39, 42, 44, 47, 50, 52, 54, 57, 60, 62, 65, 67 and 138 under conditions such that nucleic acid amplification occurs to yield an amplicon; and b) contacting the amplicon with one or more probes comprising one or more sequences selected from the group consisting of: SEQ ID NOS: 2, 14, 16, 19, 22, 27, 29, 31, 38, 41, 46, 49, 56, 59, 61, 64, and 69 under conditions such that hybridization of the probe to the amplicon occurs; wherein the hybridization of the probe is indicative of C. Difficile in the sample. In a particular embodiment, the C. Difficile gene detected is tcdB (toxin B), and/or tcdA (toxin A), and/or cdtB (binary toxin).

One embodiment is directed to a method for identifying the causative agent of colitis (abdominal pain) by detecting one or more of the toxin genes of a C. Difficile species, the method comprising: a) contacting the sample with at least one forward primer comprising the sequence selected from the group consisting of: SEQ ID NOS: 1, 4, 6, 8, 10, 12, 13, 18, 21, 23, 24, 26, 28, 30, 35, 36, 37, 40, 43, 45, 48, 51, 53, 55, 58, 63, 66, and 68 and at least one reverse primer comprising the sequence selected from the group consisting of: SEQ ID NOS: 3, 5, 7, 9, 11, 15, 17, 20, 25, 32, 33, 34, 39, 42, 44, 47, 50, 52, 54, 57, 60, 62, 65, 67 and 138 under conditions such that nucleic acid amplification occurs to yield an amplicon; and b) contacting the amplicon with one or more probes comprising one or more sequences selected from the group consisting of: SEQ ID NOS: 2, 14, 16, 19, 22, 27, 29, 31, 38, 41, 46, 49, 56, 59, 61, 64, and 69 under conditions such that hybridization of the probe to the amplicon occurs; wherein the hybridization of the probe is indicative of C. Difficile in the sample. In a particular embodiment, the C. Difficile genes are selected from the group consisting of: tcdB, tcdA and cdtB.

One embodiment is directed to screening and/or a screening kit for amplifying and sequencing C. Difficile sequences acquired from, for example, individuals in a medical facility and/or the community, comprising: a) at least one forward primer comprising the sequence selected from the group consisting of: SEQ ID NOS: 1, 4, 6, 8, 10, 12, 13, 18, 21, 23, 24, 26, 28, 30, 35, 36, 37, 40, 43, 45, 48, 51, 53, 55, 58, 63, 66, and 68; b) at least one reverse primer comprising the sequence selected from the group consisting of: SEQ ID NOS: 3, 5, 7, 9, 11, 15, 17, 20, 25, 32, 33, 34, 39, 42, 44, 47, 50, 52, 54, 57, 60, 62, 65, 67 and 138; c) reagents for the sequencing of amplified DNA fragments; and d) an internal control and a positive control. In a particular embodiment, the kit further comprises reagents for quantitating C. Difficile sequences in the sample.

### DETAILED DESCRIPTION

The pathogenicity of C. Difficile is associated with the production of two large toxins: toxin A (tcdA, 308 kD) and toxin B (tcdB, 270 kD). Both have a C-terminal receptor-binding domain, a central hydrophobic domain that is believed to mediate the insertion of the toxin into the membrane of the endosome, thereby allowing the N-terminal glucosyltransferase enzymatic domain to enter the cytosol (Kelly et al., N. Engl. J. Med. 359(18):1932-40 (2008)). Toxin A and toxin B are enterotoxic and cytotoxic in the human colon. Inside host cells, both toxins catalyze the transfer of glucose onto the Rho family of GTPases, causing disruption of the actin cytoskeleton and tight junctions, and resulting in decreased transepithelial resistance, fluid accumulation and destruction of the intestinal epithelium. Nontoxigenic strains are not pathogenic. Purified toxin A alone can induce most of the pathology observed after infection of hamsters with C. Difficile and toxin B is not toxic in animals unless it is co-administered with toxin A. However, in the context of a C. Difficile infection, toxin B is a key virulence determinant (Lyras et al., Nature. 458(7242):1176-9 (2009)). Pathogenic strains of C. Difficile producing toxin B only have been isolated from clinical samples. Toxin B has an important variant associated with Toxin A negative, Toxin B positive C. Difficile strains. (Drudy et al., Int. J. Infect. Dis. 11:5-10 (2007). This variant is a growing concern as C. Difficile strains found in hospital environments are dynamic and change over time.

Together with three additional regulatory genes (tcdC, tcdE and tcdR), tcdA and tcdB form a 19.6-kb pathogenicity locus called PaLoc (Kelly et al., N. Engl. J. Med. 359(18):1932-40 (2008)). TcdC protein appears to inhibit toxin transcription during the early, exponential-growth phase of the bacterial life cycle (Dupuy et al., J. Med. Microbiol. 57:685-689 (2008)). Some strains of C. Difficile also produce an actin-specific ADP-ribosyltransferase called binary toxin (CDT). It is unrelated to the pathogenicity locus that encodes toxins A and B. The binary toxin consists of two independent unlinked protein chains, designated CDTa (enzymatic component) and CDTb (binding component). Binary toxin may act synergistically with toxins A and B in causing severe colitis.

Described herein are optimized oligonucleotides that can act as probes and primers that, alone or in various combinations, allow for the detection, isolation, amplification, quantitation, monitoring, screening and sequencing of C. Difficile pathogens. Screening refers to a test or exam performed to find a condition before symptoms begin. Monitoring generally means to be aware of the state of a system. Nucleic acid primers and probes for detecting bacterial or derived genetic material of C. Difficile and methods for designing and optimizing the respective primer and probe sequences are described. Optimized primer and probe sets were designed to target toxin genes that are conserved within the C. Difficile genome.

The primers and probes described herein can be used, for example, to confirm suspected cases of C. Difficile-associated diseases, symptoms, disorders or conditions, e.g., watery diarrhea and colitis (abdominal pain) and to determine if the causative agent is C. Difficile containing toxin gene A, and/or toxin gene B, and/or binary toxin, in a singleplex format.

The primers and probes can also be used to diagnose a co-infection of the bacteria (in a multiplex format) or, using probe(s) to diagnose an infection by C. Difficile having genes coding for a certain toxin (e.g., A, and/or B, and/or binary toxin). Included herein are probe(s), for example, to a) decrease the chance of false positive and false negative results; and b) increase the specificity of the assay.

These oligonucleotides may also be used as part of a screening kit for detecting C. Difficile within a sample acquired from the community and/or a sample acquired from within a medical facility, such as a hospital. The individual from whom the sample is acquired may or may not be symptomatic, thus a positive result from a screen would permit the hospital or doctor to perform the appropriate preventative measures to avoid contamination of others and also determine treatment options.

The primers and probes of the present invention can be used for the detection of C. Difficile species containing the genes (1) tcdB or (2) tcdA or (3) cdtB, or combined in a multiplex format to allow detection of (1) tcdB, and/or (2) tcdA and/or (3) cdtB, without loss of assay precision or sensitivity. Furthermore, the primers and probes of the present invention can be combined with the internal control without a loss of assay sensitivity. The multiplex format option allows relative comparisons to be made between these prevalent toxins. The primers and probes described herein can be used as a diagnostic reagent for C. Difficile-associated diseases, syndromes and conditions and/or be used for screening to detect C. Difficile within a sample (i.e., whether an individual is colonized).

The probe(s) (e.g., used to detect the three different toxins of C. Difficile) described herein have the unique feature of providing a lower rate of false positive and false negative results when used in diagnostic assays.

The C. Difficile-associated colonization, complications, conditions, syndromes or diseases in mammals, e.g., humans, include, but are not limited to, watery diarrhea, abdominal pain, inflamed colon (colitis), appendicitis, small bowel enteritis, reactive arthritis, cellulitis, necrotizing fasciitis, osteomyelitis, fever, blood or pus in the stool, nausea, dehydration, loss of appetite, and weight loss.

A diagnostic test that can determine multiple C. Difficile toxins simultaneously (tcdB, tcdA, and/or cdtB) is needed, as C. Difficile is the major causative agent, for example, of watery diarrhea and colitis.

The oligonucleotides described herein, and their resulting amplicons, do not cross-react and, thus, will work together without negatively impacting either of the individual/singleplex assays. The primers and probes of the present invention also do not cross-react with DNA from the organisms specified in Table 1.

TABLE 1
Panel of organisms in in silico cross reactivity screening.

| GI | Naso/Skin | Blood |
|---|---|---|
| Bacillus cereus | Adenovirus-1 | Cytomegalovirus |
| Bacteroides fragilis | Adenovirus-7 | (Human |
| Bifidobacterium | Aspergillus fumigatus | herpesvirus 5) |
| adolescentis | Bordetella pertussis | Epstein-Barr |
| Bifidobacterium | Candida albicans | Virus (Human |
| breve | Chlamydophila pneumoniae | herpesvirus 4) |
| Campylobacter coli | Corynebacterium diptheriae | |
| Campylobacter | Corynebacterium glutamicum | |
| hominis | Haemophilus influenzae | |
| Campylobacter jejuni | Legionella pneumophila | |
| Clostridium difficile | Moraxella catarrhalis | |
| Clostridium | Mycobacterium tuberculosis | |
| perfringens | Mycoplasma pneumoniae | |
| Enterobacter | Neisseria gonorrhoeae | |
| aerogenes | Neisseria meningitides | |
| Enterobacter cloacae | Neisseria mucosa | |
| Enterococcus | Pneumocystis carinii | |
| faecalis | Pseudomonas aeruginosa | |
| Enterococcus | Streptococcus pneumoniae | |
| faecium | Streptococcus pyogenes | |
| Enterococcus | (Group A strep) | |
| faecium | Streptococcus salivarius | |
| Escherichia coli | HPV-11 plasmid | |
| Esherichia coli | HPV-6b plasmid | |
| 0157:H7 | Staphylococcus aureus | |
| Helicobacter pylori | (MRSA) | |
| Lactobacillus | Staphylococcus epidermidis | |
| acidophilus | (antibiotic resistant) | |
| Lactobacillus | Staphylococcus haemolyticus | |
| plantarum | | |
| Proteus mirabilis | | |
| Proteus vulgaris | | |
| Salmonella enterica | | |
| Shigella flexneri | | |
| Vibrio choerae | | |
| Yersinia | | |
| enterocolitica | | |

Culture-based assays are currently the definitive method of choice for the determination of the cause of C. Difficile. Real-time PCR is becoming more common for testing C. Difficile, however, many of the commercially available tests lack sensitivity and specificity. There are a few real-time PCR tests for C. Difficile, however, some of these assays have high false positive rates because they identify C. Difficile strains that carry a gene coding for a toxin, but are not actively expressing the toxin.

Table 2 demonstrates possible diagnostic outcome scenarios using the probes and primers described herein in diagnostic methods.

### Table 2.

| | Target | | | | | | | |
|---|---|---|---|---|---|---|---|---|
| | tcdB | − | + | + | − | − | + | − |
| | cdtB | − | − | − | − | + | + | − |
| | tcdA | − | − | + | + | − | + | − |
| | IC/Proc | + | +/−^{a} | +/−^{a} | +/−^{a} | +/−^{a} | +/−^{a} | − |
| | Ctrl | | | | | | | |
| Outcome | | Negative | CD with | CD with | CD with | CD with | CD with | Invalid |
| | | | tcdB | tcdA | tcdA | cdtB | tcdA and | |
| | | | | and | | | tcdB and | |
| | | | | tcdB | | | cdtB | |

CD = C. Difficile species
IC/Proc Ctrl = Internal Control or Process Control
^{a}A signal indicating a high starting concentration of specific target in the absence of an internal control signal is considered to be a valid sample result

The advantages of a multiplex format of a test are: (1) simplified and improved testing and analysis; (2) increased efficiency and cost-effectiveness; (3) decreased turnaround time (increased speed of reporting results); (4) increased productivity (less equipment time needed); and (5) coordination/standardization of results for patients for multiple organisms (reduces error from inter-assay variation).

Diagnosis, detection and/or screening of C. Difficile pathogens can lead to earlier and more effective treatment of a subject. The methods for diagnosing and detecting C. Difficile infection described herein can be coupled with effective treatment therapies. The antibiotics comprising metronidazole, oral vancomycin, and linezolid are often prescribed for treatment of a C. Difficile infection. Several nucleic acid diagnostic testing kits are available, but they cannot adequately identify the broad genetic diversity of target C. Difficile strains, specifically whether the strain has toxin B, and/or toxin A, and/or binary toxin.

There is a particular need for a screening kit including oligonucleotides that may be used for detecting C. Difficile within a sample acquired from the community and/or a sample acquired from within a medical facility, such as a hospital. The treatments for C. Difficile infection will depend upon the clinical disease state of the patient, as determinable by one of ordinary skill in the art.

The present invention therefore provides a method for specifically detecting the presence of a C. Difficile pathogen in a given sample using the primers and probes provided herein. Of particular interest in this regard is the ability of the disclosed primers and probes, as well as those that can be designed according to the disclosed methods, to specifically detect all or a majority of presently characterized strains of C. Difficile. The optimized primers and probes are useful, therefore, for identifying and diagnosing the causative or contributing agents of disease caused by a C. Difficile pathogen, whereupon an appropriate treatment can then be administered to the individual to eradicate the bacteria.

The present invention provides one or more sets of primers that can anneal to all currently identified strains of the species C. Difficile and thereby amplify a target from a biological sample. The present invention provides, for example, at least a first primer and at least a second primer for C. Difficile, each of which comprises a nucleotide sequence designed according to the inventive principles disclosed herein, which are used together to amplify DNA from C. Difficile in a sample in a singleplex assay, or C. Difficile in a sample in a multiplex assay, regardless of the actual nucleotide composition of the infecting bacterial strain(s).

Also provided herein are probes that hybridize to the C. Difficile sequences and/or amplified products derived from the C. Difficile sequences. A probe can be labeled, for example, such that when it binds to an amplified or unamplified target sequence, or after it has been cleaved after binding, a fluorescent signal is emitted that is detectable under various spectroscopy and light measuring apparatuses. The use of a labeled probe, therefore, can enhance the sensitivity of detection of a target in an amplification reaction of C. Difficile sequences because it permits the detection of bacterial-derived DNA at low template concentrations that might not be conducive to visual detection as a gel-stained amplification product.

Primers and probes are sequences that anneal to a bacterial genomic or bacterial genomic derived sequence, e.g., C. Difficile sequences, e.g., tcdB, and/or tcdA, and/or cdtB toxin sequences (the “target” sequences). The target sequence can be, for example, a bacterial genome or a subset, “region”, of, in this case, a bacterial genome. In one embodiment, the entire genomic sequence can be “scanned” for optimized primers and probes useful for detecting bacterial strains. In other embodiments, particular regions of the bacterial genome can be scanned, e.g., regions that are documented in the literature as being useful for detecting multiple strains, regions that are conserved, or regions where sufficient information is available in, for example, a public database, with respect to bacterial strains.

Sets or groups of primers and probes are generated based on the target to be detected. The set of all possible primers and probes can include, for example, sequences that include the variability at every site based on the known bacterial strains, or the primers and probes can be generated based on a consensus sequence of the target. The primers and probes are generated such that the primers and probes are able to anneal to a particular strain or sequence under high stringency conditions. For example, one of ordinary skill in the art recognizes that for any particular sequence, it is possible to provide more than one oligonucleotide sequence that will anneal to the particular target sequence, even under high stringency conditions. The set of primers and probes to be sampled includes, for example, all such oligonucleotides for all bacterial strain sequences. Alternatively, the primers and probes include all such oligonucleotides for a given consensus sequence for a target.

Typically, stringent hybridization and washing conditions are used for nucleic acid molecules over about 500 bp. Stringent hybridization conditions include a solution comprising about 1 M Na⁻ at 25° C. to 30° C. below the Tm; e.g., 5× SSPE, 0.5% SDS, at 65° C.; (see, Ausubel, et al., Current Protocols in Molecular Biology, Greene Publishing, 1995; Sambrook et al., Molecular Cloning: A Laboratory Manual, Cold Spring Harbor Press, 1989). Tm is dependent on both the G+C content and the concentration of salt ions, e.g., Na⁺ and K^{-.} A formula to calculate the Tm of nucleic acid molecules greater than about 500 by is Tm=81.5+0.41(%(G+C))−log₁₀[Na⁺]. Washing conditions are generally performed at least at equivalent stringency conditions as the hybridization. If the background levels are high, washing can be performed at higher stringency, such as around 15° C. below the Tm.

The set of primers and probes, once determined as described above, are optimized for hybridizing to a plurality of bacterial strains by employing scoring and/or ranking steps that provide a positive or negative preference or “weight” to certain nucleotides in a target nucleic acid strain sequence. If a consensus sequence is used to generate the full set of primers and probes, for example, then a particular primer sequence is scored for its ability to anneal to the corresponding sequence of every known native strain sequence. Even if a probe were originally generated based on a consensus, therefore, the validation of the probe is in its ability to specifically anneal and detect every, or a large majority of, bacterial strain sequences. The particular scoring or ranking steps performed depend upon the intended use for the primer and/or probe, the particular target nucleic acid sequence, and the number of strains of that target nucleic acid sequence. The methods of the invention provide optimal primer and probe sequences because they hybridize to all or a subset of strains of the species C. Difficile. Once optimized oligonucleotides are identified that can anneal to bacterial strains, the sequences can then further be optimized for use, for example, in conjunction with another optimized sequence as a “primer set” or for use as a probe. A “primer set” is defined as at least one forward primer and one reverse primer.

Described herein are methods for using the C. Difficile primers and probes for producing a nucleic acid product, for example, comprising contacting one or more nucleic acid sequences of SEQ ID NOS: 1-69 and 138 to a sample comprising at least one of the strains of C. Difficile under conditions suitable for nucleic acid polymerization. The primers and probes can additionally be used to quantitate and/or sequence C. Difficile sequences, or used as a diagnostic to, for example, detect C. Difficile in a sample, e.g., obtained from a subject, e.g., a mammalian subject. The primers and probes can additionally be used to screen for C. Difficile in a sample. Particular combinations for amplifying C. Difficile sequences include, for example, using at least one forward primer selected from the group consisting of SEQ ID NOS: 1, 4, 6, 8, 10, 12, 13, 18, 21, 23, 24, 26, 28, 30, 35, 36, 37, 40, 43, 45, 48, 51, 53, 55, 58, 63, 66, and 68 and at least one reverse primer selected from the group consisting of SEQ ID NOS: 3, 5, 7, 9, 11, 15, 17, 20, 25, 32, 33, 34, 39, 42, 44, 47, 50, 52, 54, 57, 60, 62, 65, 67 and 138.

Methods are described for detecting C. Difficile pathogens in a sample, for example, comprising (1) contacting at least one forward and reverse primer set, e.g., SEQ ID NOS 1, 4, 6, 8, 10, 12, 13, 18, 21, 23, 24, 26, 28, 30, 35, 36, 37, 40, 43, 45, 48, 51, 53, 55, 58, 63, 66, and 68 (forward primers) and SEQ ID NOS: 3, 5, 7, 9, 11, 15, 17, 20, 25, 32, 33, 34, 39, 42, 44, 47, 50, 52, 54, 57, 60, 62, 65, 67 and 138 (reverse primers) to a sample; (2) conducting an amplification; and (3) detecting the generation of an amplified product, wherein the generation of an amplified product indicates the presence of C. Difficile in the sample.

The detection of amplicons using probes described herein can be performed, for example, using a labeled probe, e.g., the probe comprising a nucleotide sequence selected from the group consisting of: SEQ ID NOS: 2, 14, 16, 19, 22, 27, 29, 31, 38, 41, 46, 49, 56, 59, 61, 64, and 69, that hybridizes to one of the strands of the amplicon generated by at least one forward and reverse primer set. The probe(s) can be, for example, fluorescently labeled, thereby indicating that the detection of the probe involves measuring the fluorescence of the sample of the bound probe, e.g., after bound probes have been isolated. Probes can also be fluorescently labeled in such a way, for example, such that they only fluoresce upon hybridizing to their target, thereby eliminating the need to isolate hybridized probes. The probe can also comprise a fluorescent reporter moiety and a quencher of fluorescence moiety. Upon probe hybridization with the amplified product, the exonuclease activity of a DNA polymerase can be used to cleave the probe reporter and quencher, resulting in the unquenched emission of fluorescence, which is detected. An increase in the amplified product causes a proportional increase in fluorescence, due to cleavage of the probe and release of the reporter moiety of the probe. The amplified product is quantified in real time as it accumulates. For multiplex reactions involving more than one distinct probe, each of the probes can be labeled with a different distinguishable and detectable label.

The probes can be molecular beacons. Molecular beacons are single-stranded probes that form a stem-loop structure. A fluorophore can be, for example, covalently linked to one end of the stem and a quencher can be covalently linked to the other end of the stem forming a stem hybrid. When a molecular beacon hybridizes to a target nucleic acid sequence, the probe undergoes a conformational change that results in the dissociation of the stem hybrid and, thus the fluorophore and the quencher move away from each other, enabling the probe to fluoresce brightly. Molecular beacons can be labeled with differently colored fluorophores to detect different target sequences. Any of the probes described herein can be modified and utilized as molecular beacons.

Primer or probe sequences can be ranked according to specific hybridization parameters or metrics that assign a score value indicating their ability to anneal to bacterial strains under highly stringent conditions. Where a primer set is being scored, a “first” or “forward” primer is scored and the “second” or “reverse”-oriented primer sequences can be optimized similarly but with potentially additional parameters, followed by an optional evaluation for primer dimmers, for example, between the forward and reverse primers.

The scoring or ranking steps that are used in the methods of determining the primers and probes include, for example, the following parameters: a target sequence score for the target nucleic acid sequence(s), e.g., the PriMD® score; a mean conservation score for the target nucleic acid sequence(s); a mean coverage score for the target nucleic acid sequence(s); 100% conservation score of a portion (e.g., 5′ end, center, 3′ end) of the target nucleic acid sequence(s); a species score; a strain score; a subtype score; a serotype score; an associated disease score; a year score; a country of origin score; a duplicate score; a patent score; and a minimum qualifying score. Other parameters that are used include, for example, the number of mismatches, the number of critical mismatches (e.g., mismatches that result in the predicted failure of the sequence to anneal to a target sequence), the number of native strain sequences that contain critical mismatches, and predicted Tm values. The term “Tm” refers to the temperature at which a population of double-stranded nucleic acid molecules becomes half-dissociated into single strands. Methods for calculating the Tm of nucleic acids are known in the art (Berger and Kimmel (1987) Meth. Enzymol., Vol. 152: Guide To Molecular Cloning Techniques, San Diego: Academic Press, Inc. and Sambrook et al. (1989) Molecular Cloning: A Laboratory Manual, (2nd ed.) Vols. 1-3, Cold Spring Harbor Laboratory).

The resultant scores represent steps in determining nucleotide or whole target nucleic acid sequence preference, while tailoring the primer and/or probe sequences so that they hybridize to a plurality of target nucleic acid strains. The methods of determining the primers and probes also can comprise the step of allowing for one or more nucleotide changes when determining identity between the candidate primer and probe sequences and the target nucleic acid strain sequences, or their complements.

In another embodiment, the methods of determining the primers and probes comprise the steps of comparing the candidate primer and probe nucleic acid sequences to “exclusion nucleic acid sequences” and then rejecting those candidate nucleic acid sequences that share identity with the exclusion nucleic acid sequences. In another embodiment, the methods comprise the steps of comparing the candidate primer and probe nucleic acid sequences to “inclusion nucleic acid sequences” and then rejecting those candidate nucleic acid sequences that do not share identity with the inclusion nucleic acid sequences.

In other embodiments of the methods of determining the primers and probes, optimizing primers and probes comprises using a polymerase chain reaction (PCR) penalty score formula comprising at least one of a weighted sum of: primer Tm—optimal Tm; difference between primer Tms; amplicon length—minimum amplicon length; and distance between the primer and a TaqMan® probe. The optimizing step also can comprise determining the ability of the candidate sequence to hybridize with the most target nucleic acid strain sequences (e.g., the most target organisms or genes). In another embodiment, the selecting or optimizing step comprises determining which sequences have mean conservation scores closest to 1, wherein a standard of deviation on the mean conservation scores is also compared.

In other embodiments, the methods further comprise the step of evaluating which target nucleic acid strain sequences are hybridized by an optimal forward primer and an optimal reverse primer, for example, by determining the number of base differences between target nucleic acid strain sequences in a database. For example, the evaluating step can comprise performing an in silico polymerase chain reaction, involving (1) rejecting the forward primer and/or reverse primer if it does not meet inclusion or exclusion criteria; (2) rejecting the forward primer and/or reverse primer if it does not amplify a medically valuable nucleic acid; (3) conducting a BLAST analysis to identify forward primer sequences and/or reverse primer sequences that overlap with a published and/or patented sequence; (4) and/or determining the secondary structure of the forward primer, reverse primer, and/or target. In an embodiment, the evaluating step includes evaluating whether the forward primer sequence, reverse primer sequence, and/or probe sequence hybridizes to sequences in the database other than the nucleic acid sequences that are representative of the target strains.

The present invention provides oligonucleotides that have preferred primer and probe qualities. These qualities are specific to the sequences of the optimized probes; however, one of ordinary skill in the art would recognize that other molecules with similar sequences could also be used. The oligonucleotides provided herein comprise a sequence that shares at least about 60-70% identity with a sequence described in Tables 4-6. In addition, the sequences can be incorporated into longer sequences, provided they function to specifically anneal to and identify bacterial strains. In another embodiment, the invention provides a nucleic acid comprising a sequence that shares at least about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identity with the sequences of Tables 4-6 or complement thereof. The terms “homology” or “identity” or “similarity” refer to sequence relationships between two nucleic acid molecules and can be determined by comparing a nucleotide position in each sequence when aligned for purposes of comparison. The term “homology” refers to the relatedness of two nucleic acid or protein sequences. The term “identity” refers to the degree to which nucleic acids are the same between two sequences. The term “similarity” refers to the degree to which nucleic acids are the same, but includes neutral degenerate nucleotides that can be substituted within a codon without changing the amino acid identity of the codon, as is well known in the art. The primer and/or probe nucleic acid sequences of the invention are complementary to the target nucleic acid sequence. The probe and/or primer nucleic acid sequences of the invention are optimal for identifying numerous strains of a target nucleic acid, e.g., from pathogens of the species C. Difficile. In an embodiment, the nucleic acids of the invention are primers for the synthesis (e.g., amplification) of target nucleic acid strains and/or probes for identification, isolation, detection, quantitation or analysis of target nucleic acid strains, e.g., an amplified target nucleic acid strain that is amplified using the primers of the invention.

The present oligonucleotides hybridize with more than one bacterial strain (strains as determined by differences in their genomic sequence). The probes and primers provided herein can, for example, allow for the detection and quantitation of currently identified bacterial strains or a subset thereof. In addition, the primers and probes of the present invention, depending on the strain sequence(s), can allow for the detection and quantitation of previously unidentified bacterial strains. In addition, the primers and probes of the present invention, depending on the strain sequence(s), can allow for the detection and quantitation of previously unknown bacterial strains. The methods of the invention provide for optimal primers and probes, and sets thereof, and combinations of sets thereof, which can hybridize with a larger number of target strains than available primers and probes.

In other aspects, the invention also provides vectors (e.g., plasmid, phage, expression), cell lines (e.g., mammalian, insect, yeast, bacterial), and kits comprising any of the sequences of the invention described herein. The invention further provides known or previously unknown target nucleic acid strain sequences that are identified, for example, using the methods of the invention. In an embodiment, the target nucleic acid strain sequence is an amplification product. In another embodiment, the target nucleic acid strain sequence is a native or synthetic nucleic acid. The primers, probes, target nucleic acid strain sequences, vectors, cell lines, and kits can have any number of uses, such as diagnostic, investigative, confirmatory, monitoring, predictive or prognostic.

Diagnostic kits that comprise one or more of the oligonucleotides described herein, which are useful for detecting C. Difficile infection in an individual and/or from a sample, are provided herein. An individual can be a human male, human female, human adult, human child, or human fetus. An individual can also be any mammal, reptile, avian, fish, or amphibian. Hence, an individual can be a primate, pig, horse, cattle, sheep, dog, rabbit, guinea pig, rodent, bird or fish. A sample includes any item, surface, material, clothing, or environment, for example, sewage or water treatment plants, in which it may be desirable to test for the presence of C. Difficile strains. Thus, for instance, the present invention includes testing door handles, faucets, table surfaces, elevator buttons, chairs, toilet seats, sinks, kitchen surfaces, children's cribs, bed linen, pillows, keyboards, and so on, for the presence of C. Difficile strains.

A probe of the present invention can comprise a label such as, for example, a fluorescent label, a chemiluminescent label, a radioactive label, biotin, mass tags, gold, dendrimers, aptamer, enzymes, proteins, quenchers and molecular motors. The probe may also be labeled with other similar detectable labels used in conjunction with probe technology as known by one of ordinary skill in the art. In an embodiment, the probe is a hydrolysis probe, such as, for example, a TaqMan® probe. In other embodiments, the probes of the invention are molecular beacons, any fluorescent probes, and probes that are replaced by any double stranded DNA binding dyes.

Oligonucleotides of the present invention do not only include primers that are useful for conducting the aforementioned amplification reactions, but also include oligonucleotides that are attached to a solid support, such as, for example, a microarray, multiwell plate, column, bead, glass slide, polymeric membrane, glass microfiber, plastic tubes, cellulose, and carbon nanostructures. Hence, detection of C. Difficile strains can be performed by exposing such an oligonucleotide-covered surface to a sample such that the binding of a complementary strain DNA sequence to a surface-attached oligonucleotide elicits a detectable signal or reaction.

Oligonucleotides of the present invention also include primers for isolating, quantitating and sequencing nucleic acid sequences derived from any identified or yet to be isolated and identified C. Difficile genome.

One embodiment of the invention uses solid support-based oligonucleotide hybridization methods to detect gene expression. Solid support-based methods suitable for practicing the present invention are widely known and are described (PCT application WO 95/11755; Huber et al., Anal. Biochem., 299:24, 2001; Meiyanto et al., Biotechniques, 31:406, 2001; Relogio et al., Nucleic Acids Res., 30:e51, 2002; the contents of which are incorporated herein by reference in their entirety). Any solid surface to which oligonucleotides can be bound, covalently or non-covalently, may be used. Such solid supports include, but are not limited to, filters, polyvinyl chloride dishes, silicon or glass based chips.

In certain embodiments, the nucleic acid molecule can be directly bound to the solid support or bound through a linker arm, which is typically positioned between the nucleic acid sequence and the solid support. A linker arm that increases the distance between the nucleic acid molecule and the substrate can increase hybridization efficiency. There are a number of ways to position a linker arm. In one common approach, the solid support is coated with a polymeric layer that provides linker arms with a plurality of reactive ends/sites. A common example of this type is glass slides coated with polylysine (U.S. Pat. No. 5,667,976, the contents of which are incorporated herein by reference in its entirety), which are commercially available. Alternatively, the linker arm can be synthesized as part of or conjugated to the nucleic acid molecule, and then this complex is bonded to the solid support. One approach, for example, takes advantage of the extremely high affinity biotin-streptavidin interaction. The streptavidin-biotinylated reaction is stable enough to withstand stringent washing conditions and is sufficiently stable that it is not cleaved by laser pulses used in some detection systems, such as matrix-assisted laser desorption/ionization time of flight (MALDI-TOF) mass spectrometry. Therefore, streptavidin can be covalently attached to a solid support, and a biotinylated nucleic acid molecule will bind to the streptavidin-coated surface. In one version of this method, an amino-coated silicon wafer is reacted with the n-hydroxysuccinimido-ester of biotin and complexed with streptavidin. Biotinylated oligonucleotides are bound to the surface at a concentration of about 20 fmol DNA per mm².

One can alternatively directly bind DNA to the support using carbodiimides, for example. In one such method, the support is coated with hydrazide groups, and then treated with carbodiimide. Carboxy-modified nucleic acid molecules are then coupled to the treated support. Epoxide-based chemistries are also being employed with amine modified oligonucleotides. Other chemistries for coupling nucleic acid molecules to solid substrates are known to those of one of ordinary skill in the art.

The nucleic acid molecules, e.g., the primers and probes of the present invention, must be delivered to the substrate material, which is suspected of containing or is being tested for the presence and number of C. Difficile molecules. Because of the miniaturization of the arrays, delivery techniques must be capable of positioning very small amounts of liquids in very small regions, very close to one another and amenable to automation. Several techniques and devices are available to achieve such delivery. Among these are mechanical mechanisms (e.g., arrayers from GeneticMicroSystems, MA, USA) and ink jet technology. Very fine pipets can also be used.

Other formats are also suitable within the context of this invention. For example, a 96-well format with fixation of the nucleic acids to a nitrocellulose or nylon membrane can also be employed.

After the nucleic acid molecules have been bound to the solid support, it is often useful to block reactive sites on the solid support that are not consumed in binding to the nucleic acid molecule. In the absence of the blocking step, excess primers and/or probes can, to some extent, bind directly to the solid support itself, giving rise to non-specific binding. Non-specific binding can sometimes hinder the ability to detect low levels of specific binding. A variety of effective blocking agents (e.g., milk powder, serum albumin or other proteins with free amine groups, polyvinylpyrrolidine) can be used and others are known to those skilled in the art (U.S. Pat. No. 5,994,065, the contents of which are incorporated herein by reference in their entirety). The choice depends at least in part upon the binding chemistry.

One embodiment uses oligonucleotide arrays, e.g., microarrays that can be used to simultaneously observe the expression of a number of C. Difficile strain genes. Oligonucleotide arrays comprise two or more oligonucleotide probes provided on a solid support, wherein each probe occupies a unique location on the support. The location of each probe can be predetermined, such that detection of a detectable signal at a given location is indicative of hybridization to an oligonucleotide probe of a known identity. Each predetermined location can contain more than one molecule of a probe, but each molecule within the predetermined location has an identical sequence. Such predetermined locations are termed features. There can be, for example, from 2, 10, 100, 1,000, 2,000 or 5,000 or more of such features on a single solid support. In one embodiment, each oligonucleotide is located at a unique position on an array at least 2, at least 3, at least 4, at least 5, at least 6, or at least 10 times.

Oligonucleotide probe arrays for detecting gene expression can be made and used according to conventional techniques described (Lockhart et al., Nat. Biotech., 14:1675-1680, 1996; McGall et al., Proc. Natl. Acad. Sci. USA, 93:13555, 1996; Hughes et al., Nat. Biotechnol., 19:342, 2001). A variety of oligonucleotide array designs are suitable for the practice of this invention.

Generally, a detectable molecule, also referred to herein as a label, can be incorporated or added to an array's probe nucleic acid sequences. Many types of molecules can be used within the context of this invention. Such molecules include, but are not limited to, fluorochromes, chemiluminescent molecules, chromogenic molecules, radioactive molecules, mass spectrometry tags, proteins, and the like. Other labels will be readily apparent to one skilled in the art.

Oligonucleotide probes used in the methods of the present invention, including microarray techniques, can be generated using PCR. PCR primers used in generating the probes are chosen, for example, based on the sequences of Tables 4-6. In one embodiment, oligonucleotide control probes also are used. Exemplary control probes can fall into at least one of three categories referred to herein as (1) normalization controls, (2) expression level controls and (3) negative controls. In microarray methods, one or more of these control probes can be provided on the array with the inventive cell cycle gene-related oligonucleotides.

Normalization controls correct for dye biases, tissue biases, dust, slide irregularities, malformed slide spots, etc. Normalization controls are oligonucleotide or other nucleic acid probes that are complementary to labeled reference oligonucleotides or other nucleic acid sequences that are added to the nucleic acid sample to be screened. The signals obtained from the normalization controls, after hybridization, provide a control for variations in hybridization conditions, label intensity, reading efficiency and other factors that can cause the signal of a perfect hybridization to vary between arrays. The normalization controls also allow for the semi-quantification of the signals from other features on the microarray. In one embodiment, signals (e.g., fluorescence intensity or radioactivity) read from all other probes used in the method are divided by the signal from the control probes, thereby normalizing the measurements.

Virtually any probe can serve as a normalization control. Hybridization efficiency varies, however, with base composition and probe length. Preferred normalization probes are selected to reflect the average length of the other probes being used, but they also can be selected to cover a range of lengths. Further, the normalization control(s) can be selected to reflect the average base composition of the other probe(s) being used. In one embodiment, only one or a few normalization probes are used, and they are selected such that they hybridize well (i.e., without forming secondary structures) and do not match any test probes. In one embodiment, the normalization controls are mammalian genes.

“Negative control” probes are not complementary to any of the test oligonucleotides (i.e., the inventive cell cycle gene-related oligonucleotides), normalization controls, or expression controls. In one embodiment, the negative control is a mammalian gene which is not complementary to any other sequence in the sample.

The terms “background” and “background signal intensity” refer to hybridization signals resulting from non-specific binding or other interactions between the labeled target nucleic acids (e.g., mRNA present in the biological sample) and components of the oligonucleotide array. Background signals also can be produced by intrinsic fluorescence of the array components themselves. A single background signal can be calculated for the entire array, or a different background signal can be calculated for each target nucleic acid. In one embodiment, background is calculated as the average hybridization signal intensity for the lowest 5 to 10 percent of the oligonucleotide probes being used, or, where a different background signal is calculated for each target gene, for the lowest 5 to 10 percent of the probes for each gene. Where the oligonucleotide probes corresponding to a particular C. Difficile target hybridize well and, hence, appear to bind specifically to a target sequence, they should not be used in a background signal calculation. Alternatively, background can be calculated as the average hybridization signal intensity produced by hybridization to probes that are not complementary to any sequence found in the sample (e.g., probes directed to nucleic acids of the opposite sense or to genes not found in the sample). In microarray methods, background can be calculated as the average signal intensity produced by regions of the array that lack any oligonucleotides probes at all.

In an alternative embodiment, the nucleic acid molecules are directly or indirectly coupled to an enzyme. Following hybridization, a chromogenic substrate is applied and the colored product is detected by a camera, such as a charge-coupled camera. Examples of such enzymes include alkaline phosphatase, horseradish peroxidase and the like. The invention also provides methods of labeling nucleic acid molecules with cleavable mass spectrometry tags (CMST; U.S. Patent Application No: 60/279,890). After an assay is complete, and the uniquely CMST-labeled probes are distributed across the array, a laser beam is sequentially directed to each member of the array. The light from the laser beam both cleaves the unique tag from the tag-nucleic acid molecule conjugate and volatilizes it. The volatilized tag is directed into a mass spectrometer. Based on the mass spectrum of the tag and knowledge of how the tagged nucleotides were prepared, one can unambiguously identify the nucleic acid molecules to which the tag was attached (WO 9905319).

The nucleic acids, primers and probes of the present invention can be labeled readily by any of a variety of techniques. When the diversity panel is generated by amplification, the nucleic acids can be labeled during the reaction by incorporation of a labeled dNTP or use of labeled amplification primer. If the amplification primers include a promoter for an RNA polymerase, a post-reaction labeling can be achieved by synthesizing RNA in the presence of labeled NTPs. Amplified fragments that were unlabeled during amplification or unamplified nucleic acid molecules can be labeled by one of a number of end labeling techniques or by a transcription method, such as nick-translation, random-primed DNA synthesis. Details of these methods are known to one of ordinary skill in the art and are set out in methodology books. Other types of labeling reactions are performed by denaturation of the nucleic acid molecules in the presence of a DNA-binding molecule, such as RecA, and subsequent hybridization under conditions that favor the formation of a stable RecA-incorporated DNA complex.

In another embodiment, PCR-based methods are used to detect gene expression. These methods include reverse-transcriptase-mediated polymerase chain reaction (RT-PCR) including real-time and endpoint quantitative reverse-transcriptase-mediated polymerase chain reaction (Q-RTPCR). These methods are well known in the art. For example, methods of quantitative PCR can be carried out using kits and methods that are commercially available from, for example, Applied BioSystems and Stratagene®. See also Kochanowski, Quantitative PCR Protocols (Humana Press, 1999); Innis et al., supra.; Vandesompele et al., Genome Biol., 3:RESEARCH0034, 2002; Stein, Cell Mol. Life Sci. 59:1235, 2002.

The forward and reverse amplification primers and internal hybridization probe is designed to hybridize specifically and uniquely with one nucleotide sequence derived from the transcript of a target gene. In one embodiment, the selection criteria for primer and probe sequences incorporates constraints regarding nucleotide content and size to accommodate TaqMan® requirements. SYBR Green® can be used as a probe-less Q-RTPCR alternative to the TaqMan®-type assay, discussed above (ABI Prism® 7900 Sequence Detection System User Guide Applied Biosystems, chap. 1-8, App. A-F. (2002)). This device measures changes in fluorescence emission intensity during PCR amplification. The measurement is done in “real time,” that is, as the amplification product accumulates in the reaction. Other methods can be used to measure changes in fluorescence resulting from probe digestion. For example, fluorescence polarization can distinguish between large and small molecules based on molecular tumbling (U.S. Pat. No. 5,593,867).

The primers and probes of the present invention may anneal to or hybridize to various C. Difficile genetic material or genetic material derived therefrom, such as RNA, DNA, cDNA, or a PCR product.

A “sample” that is tested for the presence of C. Difficile strains includes, but is not limited to a tissue sample, such as, for example, blood, serum, plasma, enriched peripheral blood mononuclear cells, fecal material, urine, neoplastic or other tissue obtained from biopsies, cerebrospinal fluid, saliva, fluids collected from the ear, eye, mouth, and respiratory airways, sputum, stool, skin, gastric secretions, oropharyngeal swabs, nasopharyngeal swabs, throat swabs, rectal swabs, nasal aspirates, nasal wash, renal tissue, and fluid therefrom including perfusion media, pure cultures of bacterial fungal isolates, fluids and cells obtained by the perfusion of tissues of both human and animal origin, and fluids and cells derived from the culturing of human cells, including human stem cells and human cartilage or fibroblasts, pure cultures of bacterial fungal isolates, and swabs or washes of environmental surfaces, or other samples derived from environmental surfaces. In a particular embodiment, the sample is from a human, is non-human in origin, or is derived from an inanimate object. The tissue sample may be fresh, fixed, preserved, or frozen. A sample also includes any item, surface, material, or clothing, or environment, for example, sewage or water treatment plants, in which it may be desirable to test for the presence of C. Difficile strains. Thus, for instance, the present invention includes testing door handles, faucets, table surfaces, elevator buttons, chairs, toilet seats, sinks, kitchen surfaces, children's cribs, bed linen, pillows, keyboards, and so on, for the presence of C. Difficile strains.

The target nucleic acid strain that is amplified may be RNA or DNA or a modification thereof. Thus, the amplifying step can comprise isothermal or non-isothermal reactions, such as polymerase chain reaction, Scorpion® primers, molecular beacons, SimpleProbes®, HyBeacons®, cycling probe technology, Invader Assay, self-sustained sequence replication, nucleic acid sequence-based amplification, ramification amplifying method, hybridization signal amplification method, rolling circle amplification, multiple displacement amplification, thermophilic strand displacement amplification, transcription-mediated amplification, ligase chain reaction, signal mediated amplification of RNA, split promoter amplification, Q-Beta replicase, isothermal chain reaction, one cut event amplification, loop-mediated isothermal amplification, molecular inversion probes, ampliprobe, headloop DNA amplification, and ligation activated transcription. The amplifying step can be conducted on a solid support, such as a multiwell plate, array, column, bead, glass slide, polymeric membrane, glass microfiber, plastic tubes, cellulose, and carbon nanostructures. The amplifying step also comprises in situ hybridization. The detecting step can comprise gel electrophoresis, fluorescence resonant energy transfer, or hybridization to a labeled probe, such as a probe labeled with biotin, at least one fluorescent moiety, an antigen, a molecular weight tag, and a modifier of probe Tm. The detection step can also comprise the incorporation of a label (e.g., fluorescent or radioactive) during an extension reaction. The detecting step comprises measuring fluorescence, mass, charge, and/or chemiluminescence.

The target nucleic acid strain may not need amplification and may be RNA or DNA or a modification thereof. If amplification is not necessary, the target nucleic acid strain can be denatured to enable hybridization of a probe to the target nucleic acid sequence.

Hybridization may be detected in a variety of ways and with a variety of equipment. In general, the methods can be categorized as those that rely upon detectable molecules incorporated into the diversity panels and those that rely upon measurable properties of double-stranded nucleic acids (e.g., hybridized nucleic acids) that distinguish them from single-stranded nucleic acids (e.g., unhybridized nucleic acids). The latter category of methods includes intercalation of dyes, such as, for example, ethidium bromide, into double-stranded nucleic acids, differential absorbance properties of double and single stranded nucleic acids, binding of proteins that preferentially bind double-stranded nucleic acids, and the like.

### EXEMPLIFICATION

### Example 1

### Scoring a Set of Predicted Annealing Oligonucleotides

Each of the sets of primers and probes selected is ranked by a combination of methods as individual primers and probes and as a primer/probe set. This involves one or more methods of ranking (e.g., joint ranking, hierarchical ranking, and serial ranking) where sets of primers and probes are eliminated or included based on any combination of the following criteria, and a weighted ranking again based on any combination of the following criteria, for example: (A) Percentage Identity to Target Strains; (B) Conservation Score; (C) Coverage Score; (D) Strain/Subtype/Serotype Score; (E) Associated Disease Score; (F) Duplicates Sequences Score; (G) Year and Country of Origin Score; (H) Patent Score, and (I) Epidemiology Score.

### (A) Percentage Identity

A percentage identity score is based upon the number of target nucleic acid strain (e.g., native) sequences that can hybridize with perfect conservation (the sequences are perfectly complimentary) to each primer or probe of a primer set and probe set. If the score is less than 100%, the program ranks additional primer set and probe sets that are not perfectly conserved. This is a hierarchical scale for percent identity starting with perfect complimentarity, then one base degeneracy through to the number of degenerate bases that would provide the score closest to 100%. The position of these degenerate bases would then be ranked. The methods for calculating the conservation is described under section B.

(i) Individual Base Conservation Score

A set of conservation scores is generated for each nucleotide base in the consensus sequence and these scores represent how many of the target nucleic acid strains sequences have a particular base at this position. For example, a score of 0.95 for a nucleotide with an adenosine, and 0.05 for a nucleotide with a cytidine means that 95% of the native sequences have an A at that position and 5% have a C at that position. A perfectly conserved base position is one where all the target nucleic acid strain sequences have the same base (either an A, C, G, or T/U) at that position. If there is an equal number of bases (e.g., 50% A & 50% T) at a position, it is identified with an N.

(ii) Candidate Primer/Probe Sequence Conservation

An overall conservation score is generated for each candidate primer or probe sequence that represents how many of the target nucleic acid strain sequences will hybridize to the primers or probes. A candidate sequence that is perfectly complimentary to all the target nucleic acid strain sequences will have a score of 1.0 and rank the highest. For example, illustrated below in Table 3 are three different 10-base candidate probe sequences that are targeted to different regions of a consensus target nucleic acid strain sequence. Each candidate probe sequence is compared to a total of 10 native sequences.

```
TABLE 3
#1. A A A C A C G T G C
        0.7 1.0 1.0 1.0 1.0 1.0 1.0 1.0 1.0 1.0
(SEQ ID NO: 139)
→Number of target nucleic acid strain sequences
that are perfectly complimentary-7. Three out
of the ten sequences do not have an A at
position 1.
#2. C C T T G T T C C A
        1.0 0.9 1.0 0.9 0.9 1.0 1.0 1.0 1.0 1.0
(SEQ ID NO: 140)
→Number of target nucleic acid strain sequences
that are perfectly complimentary-7, 8, or 9. At
least one target nucleic acid strain does not
have a C at position 2, T at position 4, or G
at position 5. These differences may all be on
one target nucleic acid strain molecule or may
be on two or three separate molecules.
#3. C A G G G A C G A T
        1.0 1.0 1.0 1.0 1.0 0.9 0.8 1.0 1.0 1.0
(SEQ ID NO: 141)
→Number of target nucleic acid strain sequences
that are perfectly complimentary-7 or 8. At
least one target nucleic acid strain does not
have an A at position 6 and at least two target
nucleic acid strain do not have a C at position
7. These differences may all be on one target
nucleic acid strain molecule or may be on two
separate molecules.
```

A simple arithmetic mean for each candidate sequence would generate the same value of 0.97. The number of target nucleic acid strain sequences identified by each candidate probe sequence, however, can be very different. Sequence #1 can only identify 7 native sequences because of the 0.7 (out of 1.0) score by the first base—A. Sequence #2 has three bases each with a score of 0.9; each of these could represent a different or shared target nucleic acid strain sequence. Consequently, Sequence #2 can identify 7, 8 or 9 target nucleic acid strain sequences. Similarly, Sequence #3 can identify 7 or 8 of the target nucleic acid strain sequences. Sequence #2 would, therefore, be the best choice if all the three bases with a score of 0.9 represented the same 9 target nucleic acid strain sequences.

(iii) Overall Conservation Score of the Primer and Probe Set—Percent Identity

The same method described in (ii) when applied to the complete primer set and probe set will generate the percent identity for the set (see A above). For example, using the same sequences illustrated above, if Sequences #1 and #2 are primers and Sequence #3 is a probe, then the percent identity for the target can be calculated from how many of the target nucleic acid strain sequences are identified with perfect complimentarity by all three primer/probe sequences. The percent identity could be no better than 0.7 (7 out of 10 target nucleic acid strain sequences) but as little as 0.1 if each of the degenerate bases reflects a different target nucleic acid strain sequence. Again, an arithmetic mean of these three sequences would be 0.97. As none of the above examples were able to capture all the target nucleic acid strain sequences because of the degeneracy (scores of less than 1.0), the ranking system takes into account that a certain amount of degeneracy can be tolerated under normal hybridization conditions, for example, during a polymerase chain reaction. The ranking of these degeneracies is described in (iv) below.

An in silico evaluation determines how many native sequences (e.g., original sequences submitted to public databases) are identified by a given candidate primer/probe set. The ideal candidate primer/probe set is one that can perform PCR and the sequences are perfectly complimentary to all the known native sequences that were used to generate the consensus sequence. If there is no such candidate, then the sets are ranked according to how many degenerate bases can be accepted and still hybridize to only the target sequence during the PCR and yet identify all the native sequences.

The hybridization conditions, for TaqMan® as an example, are: 10-50 mM Tris-HCl pH 8.3, 50 mM KCl, 0.1-0.2% Triton® X-100 or 0.1% Tween®, 1-5 mM MgCl₂. The hybridization is performed at 58-60° C. for the primers and 68-70° C. for the probe. The in silico PCR identifies native sequences that are not amplifiable using the candidate primers and probe set. The rules can be as simple as counting the number of degenerate bases to more sophisticated approaches based on exploiting the PCR criteria used by the PriMD® software. Each target nucleic acid strain sequence has a value or weight (see Score assignment above). If the failed target nucleic acid strain sequence is medically valuable, the primer/probe set is rejected. This in silico analysis provides a degree of confidence for a given genotype and is important when new sequences are added to the databases. New target nucleic acid strain sequences are automatically entered into both the “include” and “exclude” categories. Published primer and probes will also be ranked by the PriMD software.

The PriMD® software provides comprehensive analysis of all known target sequences to design primers and probes with the best possible sensitivity and specificity. In addition, PriMD software facilitates design of multiplex real-time PCR tests, where compatibility and performance of the separate reagent sets is important and can be used together in the same reaction. Using PriMD, optimal TaqMan primer and probe sets can be designed to target conserved regions of the tcdA, tcdB, and binary toxin genes that are known to be in certain C. Difficile strains.

The PriMD® software generated TaqMan primer and probe candidates that detect tcdA, tcdB, and binary toxin genes. PriMD analyzes all available sequences from a GenBank for these genes, and selected primer and probe sets with the highest predicted specificity and sensitivity. The weighted distribution of oligo sets also includes length, amplicon size, Tm, and other oligo sequence characteristics (e.g., repetitive sequences, presence of a 3′ clamp).

(iv) Position (5′ to 3′) of the Base Conservation Score

In an embodiment, primers do not have bases in the terminal five positions at the 3′ end with a score less than 1. This is one of the last parameters to be relaxed if the method fails to select any candidate sequences. The next best candidate having a perfectly conserved primer would be one where the poorer conserved positions are limited to the terminal bases at the 5′ end. The closer the poorer conserved position is to the 5′ end, the better the score. For probes, the position criteria are different. For example, with a TaqMan® probe, the most destabilizing effect occurs in the center of the probe. The 5′ end of the probe is also important as this contains the reporter molecule that must be cleaved, following hybridization to the target, by the polymerase to generate a sequence-specific signal. The 3′ end is less critical. Therefore, a sequence with a perfectly conserved middle region will have the higher score. The remaining ends of the probe are ranked in a similar fashion to the 5′ end of the primer. Thus, the next best candidate to a perfectly conserved TaqMan® probe would be one where the poorer conserved positions are limited to the terminal bases at either the 5′ or 3′ ends. The hierarchical scoring will select primers with only one degeneracy first, then primers with two degeneracies next and so on. The relative position of each degeneracy will then be ranked favoring those that are closest to the 5′ end of the primers and those closest to the 3′ end of the TaqMan® probe. If there are two or more degenerate bases in a primer and probe set, the ranking will initially select the sets where the degeneracies occur on different sequences.

### B. Coverage Score

The total number of aligned sequences is considered under a coverage score. A value is assigned to each position based on how many times that position has been reported or sequenced. Alternatively, coverage can be defined as how representative the sequences are of the known strains, subtypes etc., or their relevance to a certain diseases. For example, the target nucleic acid strain sequences for a particular gene may be very well conserved and show complete coverage but certain strains are not represented in those sequences.

A sequence is included if it aligns with any part of the consensus sequence, which is usually a whole gene or a functional unit, or has been described as being a representative of this gene. Even though a base position is perfectly conserved it may only represent a fraction of the total number of sequences (for example, if there are very few sequences). For example, region A of a gene shows a 100% conservation from 20 sequence entries while region B in the same gene shows a 98% conservation but from 200 sequence entries. There is a relationship between conservation and coverage if the sequence shows some persistent variability. As more sequences are aligned, the conservation score falls, but this effect is lessened as the number of sequences gets larger. Unless the number of sequences is very small (e.g., under 10) the value of the coverage score is small compared to that of the conservation score. To obtain the best consensus sequence, artificial spaces are allowed to be introduced. Such spaces are not considered in the coverage score.

### C. Strain/Subtype/Serotype Score

A value is assigned to each strain or subtype or serotype based upon its relevance to a disease. For example, strains of C. Difficile that are linked to high frequencies of infection will have a higher score than strains that are generally regarded as benign. The score is based upon sufficient evidence to automatically associate a particular strain with a disease.

### D. Associated Disease Score

The associated disease score pertains to strains that are not known to be associated with a particular disease (to differentiate from D above). Here, a value is assigned only if the submitted sequence is directly linked to the disease and that disease is pertinent to the assay.

### E. Duplicate Sequences Score

If a particular sequence has been sequenced more than once it will have an effect on representation, for example, a strain that is represented by 12 entries in GenBank of which six are identical and the other six are unique. Unless the identical sequences can be assigned to different strains/subtypes (usually by sequencing other genes or by immunology methods) they will be excluded from the scoring.

### F. Year and Country of Origin Score

The year and country of origin scores are important in terms of the age of the human population and the need to provide a product for a global market. For example, strains identified or collected many years ago may not be relevant today. Furthermore, it is probably difficult to obtain samples that contain these older strains. Certain divergent strains from more obscure countries or sources may also be less relevant to the locations that will likely perform clinical tests, or may be more important for certain countries (e.g., North America, Europe, or Asia).

### G. Patent Score

Candidate target strain sequences published in patents are searched electronically and annotated such that patented regions are excluded. Alternatively, candidate sequences are checked against a patented sequence database.

### H. Minimum Qualifying Score

The minimum qualifying score is determined by expanding the number of allowed mismatches in each set of candidate primers and probes until all possible native sequences are represented (e.g., has a qualifying hit).

### I. Other

A score is given to based on other parameters, such as relevance to certain patients (e.g., pediatrics, immunocompromised) or certain therapies (e.g., target those strains that respond to treatment) or epidemiology. The prevalence of an organism/strain and the number of times it has been tested for in the community can add value to the selection of the candidate sequences. If a particular strain is more commonly tested then selection of it would be more likely. Strain identification can be used to select better vaccines.

### Example 2

### Primer/Probe Evaluation

Once the candidate primers and probes have received their scores and have been ranked, they are evaluated using any of a number of methods of the invention, such as BLAST analysis and secondary structure analysis.

### A. BLAST Analysis

The candidate primer/probe sets are submitted for BLAST analysis to check for possible overlap with any published sequences that might be missed by the Include/Exclude function. It also provides a useful summary.

### B. Secondary Structure

The methods of the present invention include analysis of nucleic acid secondary structure. This includes the structures of the primers and/or probes, as well as their intended target strain sequences. The methods and software of the invention predict the optimal temperatures for annealing, but assumes that the target (e.g., RNA or DNA) does not have any significant secondary structure. For example, if the starting material is RNA, the first stage is the creation of a complimentary strand of DNA (cDNA) using a specific primer. This is usually performed at temperatures where the RNA template can have significant secondary structure thereby preventing the annealing of the primer. Similarly, after denaturation of a double stranded DNA target (for example, an amplicon after PCR), the binding of the probe is dependent on there being no major secondary structure in the amplicon.

The methods of the invention can either use this information as a criteria for selecting primers and probes or evaluate any secondary structure of a selected sequence, for example, by cutting and pasting candidate primer or probe sequences into a commercial internet link that uses software dedicated to analyzing secondary structure, such as, for example, MFOLD (Zuker et al. (1999) Algorithms and Thermodynamics for RNA Secondary Structure Prediction: A Practical Guide in RNA Biochemistry and Biotechnology, J. Barciszewski and B. F. C. Clark, eds., NATO ASI Series, Kluwer Academic Publishers).

### C. Evaluating the Primer and Probe Sequences

The methods and software of the invention may also analyze any nucleic acid sequence to determine its suitability in a nucleic acid amplification-based assay. For example, it can accept a competitor's primer set and determine the following information: (1) How it compares to the primers of the invention (e.g., overall rank, PCR and conservation ranking, etc.); (2) How it aligns to the excluded libraries (e.g., assessing cross-hybridization)—also used to compare primer and probe sets to newly published sequences; and (3) If the sequence has been previously published. This step requires keeping a database of sequences published in scientific journals, posters, and other presentations.

### Example 3

### Multiplexing

The Exclude/Include capability is ideally suited for designing multiplex reactions. The parameters for designing multiple primer and probe sets adhere to a more stringent set of parameters than those used for the initial Exclude/Include function. Each set of primers and probes, together with the resulting amplicon, is screened against the other sets that constitute the multiplex reaction. As new targets are accepted, their sequences are automatically added to the Exclude category.

The database is designed to interrogate the online databases to determine and acquire, if necessary, any new sequences relevant to the targets. These sequences are evaluated against the optimal primer/probe set. If they represent a new genotype or strain, then a multiple sequence alignment may be required.

### Example 4

### Sequences Identified for Detecting the Genes tcdB (Toxin B), and/or tcdA (Toxin A), and/or cdtB (Binary Toxin) of C. Difficile

The set of primers and probes were then scored according to the methods described herein to identify the optimized primers and probes of Tables 4-6. It should be noted that the primers, as they are sequences that anneal to a plurality of all identified or unidentified C. Difficile strains, can also be used as probes either in the presence or absence of amplification of a sample.

TABLE 4
Optimized C. Difficile Primers and Probes for Detecting tcdB Gene (toxin B)

| Group | Oligonucleotide type | SEQ ID NO: | Sequence |
|---|---|---|---|
| 1 | Forward Primer | SEQ ID NO: 1 | GATGGAATCTTGCTGGTGCAT |
| | Probe | SEQ ID NO: 2 | CCCAATCTACAGCTGTCTTTACTGAATCAGGC |
| | Reverse Primer | SEQ ID NO: 3 | TTATGGCTTCTAACTGCATCTCTT |
| | Forward Primer | SEQ ID NO: 13 | CAGAATATACCTCAGAACATTTTGACA |
| | Probe | SEQ ID NO: 14 | TCTAGTGGTGATGCCTCCATATCACCAAG |
| | Reverse Primer | SEQ ID NO: 15 | AGCCCTTGATTTATAATACCCTTACTA |
| 2 | Forward Primer | SEQ ID NO: 1 | GATGGAATCTTGCTGGTGCAT |
| | Probe | SEQ ID NO: 2 | CCCAATCTACAGCTGTCTTTACTGAATCAGGC |
| | Reverse Primer | SEQ ID NO: 3 | TTATGGCTTCTAACTGCATCTCTT |
| | Forward Primer | SEQ ID NO: 13 | CAGAATATACCTCAGAACATTTTGACA |
| | Probe | SEQ ID NO: 16 | CTAGTGGTGATGCCTCCATATCACCAAGT |
| | Reverse Primer | SEQ ID NO: 17 | CTTTCACAGAAATTAGCCCTTGAT |
| 3 | Forward Primer | SEQ ID NO: 1 | GATGGAATCTTGCTGGTGCAT |
| | Probe | SEQ ID NO: 2 | CCCAATCTACAGCTGTCTTTACTGAATCAGGC |
| | Reverse Primer | SEQ ID NO: 3 | TTATGGCTTCTAACTGCATCTCTT |
| | Forward Primer | SEQ ID NO: 18 | GAACATTTTGACATGTTAGACGAAGA |
| | Probe | SEQ ID NO: 19 | AGTGGTGATGCCTCCATATCACCAAGTG |
| | Reverse Primer | SEQ ID NO: 20 | AGCCCTTGATTTATAATACCCTTACT |
| 4 | Forward Primer | SEQ ID NO: 1 | GATGGAATCTTGCTGGTGCAT |
| | Probe | SEQ ID NO: 2 | CCCAATCTACAGCTGTCTTTACTGAATCAGGC |
| | Reverse Primer | SEQ ID NO: 3 | TTATGGCTTCTAACTGCATCTCTT |
| | Forward Primer | SEQ ID NO: 21 | TGACATGTTAGACGAAGAAGTTCAA |
| | Probe | SEQ ID NO: 22 | TGCAATTTTAACTTCTAGTGGTGATGCCTCCA |
| | Reverse Primer | SEQ ID NO: 15 | AGCCCTTGATTTATAATACCCTTACTA |
| 5 | Forward Primer | SEQ ID NO: 1 | GATGGAATCTTGCTGGTGCAT |
| | Probe | SEQ ID NO: 2 | CCCAATCTACAGCTGTCTTTACTGAATCAGGC |
| | Reverse Primer | SEQ ID NO: 3 | TTATGGCTTCTAACTGCATCTCTT |
| | Forward Primer | SEQ ID NO: 23 | ACATGTTAGACGAAGAAGTTCAAAG |
| | Probe | SEQ ID NO: 19 | AGTGGTGATGCCTCCATATCACCAAGTG |
| | Reverse Primer | SEQ ID NO: 20 | AGCCCTTGATTTATAATACCCTTACT |
| 6 | Forward Primer | SEQ ID NO: 1 | GATGGAATCTTGCTGGTGCAT |
| | Probe | SEQ ID NO: 2 | CCCAATCTACAGCTGTCTTTACTGAATCAGGC |
| | Reverse Primer | SEQ ID NO: 3 | TTATGGCTTCTAACTGCATCTCTT |
| | Forward Primer | SEQ ID NO: 24 | CATGTTAGACGAAGAAGTTCAAAGT |
| | Probe | SEQ ID NO: 16 | CTAGTGGTGATGCCTCCATATCACCAAGT |
| | Reverse Primer | SEQ ID NO: 25 | CTTTCACAGAAATTAGCCCTTGATT |
| 7 | Forward Primer | SEQ ID NO: 1 | GATGGAATCTTGCTGGTGCAT |
| | Probe | SEQ ID NO: 2 | CCCAATCTACAGCTGTCTTTACTGAATCAGGC |
| | Reverse Primer | SEQ ID NO: 3 | TTATGGCTTCTAACTGCATCTCTT |
| | Forward Primer | SEQ ID NO: 26 | AGTAGTTTTGAATCTGTTCTAGCTTCT |
| | Probe | SEQ ID NO: 27 | TAGTGGTGATGCCTCCATATCACCAAGTG |
| | Reverse Primer | SEQ ID NO: 15 | AGCCCTTGATTTATAATACCCTTACTA |
| 8 | Forward Primer | SEQ ID NO: 1 | GATGGAATCTTGCTGGTGCAT |
| | Probe | SEQ ID NO: 2 | CCCAATCTACAGCTGTCTTTACTGAATCAGGC |
| | Reverse Primer | SEQ ID NO: 3 | TTATGGCTTCTAACTGCATCTCTT |
| | Forward Primer | SEQ ID NO: 28 | TGAATCTGTTCTAGCTTCTAAGTCA |
| | Probe | SEQ ID NO: 29 | CACTTGGTGATATGGAGGCATCACCACT |
| | Reverse Primer | SEQ ID NO: 20 | AGCCCTTGATTTATAATACCCTTACT |
| 9 | Forward Primer | SEQ ID NO: 4 | ATGGAATCTTGCTGGTGCAT |
| | Probe | SEQ ID NO: 2 | CCCAATCTACAGCTGTCTTTACTGAATCAGGC |
| | Reverse Primer | SEQ ID NO: 5 | TGGCTTCTAACTGCATCTCT |
| | Forward Primer | SEQ ID NO: 13 | CAGAATATACCTCAGAACATTTTGACA |
| | Probe | SEQ ID NO: 14 | TCTAGTGGTGATGCCTCCATATCACCAAG |
| | Reverse Primer | SEQ ID NO: 15 | AGCCCTTGATTTATAATACCCTTACTA |
| 10 | Forward Primer | SEQ ID NO: 4 | ATGGAATCTTGCTGGTGCAT |
| | Probe | SEQ ID NO: 2 | CCCAATCTACAGCTGTCTTTACTGAATCAGGC |
| | Reverse Primer | SEQ ID NO: 5 | TGGCTTCTAACTGCATCTCT |
| | Forward Primer | SEQ ID NO: 13 | CAGAATATACCTCAGAACATTTTGACA |
| | Probe | SEQ ID NO: 16 | CTAGTGGTGATGCCTCCATATCACCAAGT |
| | Reverse Primer | SEQ ID NO: 17 | CTTTCACAGAAATTAGCCCTTGAT |
| 11 | Forward Primer | SEQ ID NO: 4 | ATGGAATCTTGCTGGTGCAT |
| | Probe | SEQ ID NO: 2 | CCCAATCTACAGCTGTCTTTACTGAATCAGGC |
| | Reverse Primer | SEQ ID NO: 5 | TGGCTTCTAACTGCATCTCT |
| | Forward Primer | SEQ ID NO: 18 | GAACATTTTGACATGTTAGACGAAGA |
| | Probe | SEQ ID NO: 19 | AGTGGTGATGCCTCCATATCACCAAGTG |
| | Reverse Primer | SEQ ID NO: 20 | AGCCCTTGATTTATAATACCCTTACT |
| 12 | Forward Primer | SEQ ID NO: 4 | ATGGAATCTTGCTGGTGCAT |
| | Probe | SEQ ID NO: 2 | CCCAATCTACAGCTGTCTTTACTGAATCAGGC |
| | Reverse Primer | SEQ ID NO: 5 | TGGCTTCTAACTGCATCTCT |
| | Forward Primer | SEQ ID NO: 21 | TGACATGTTAGACGAAGAAGTTCAA |
| | Probe | SEQ ID NO: 22 | TGCAATTTTAACTTCTAGTGGTGATGCCTCCA |
| | Reverse Primer | SEQ ID NO: 15 | AGCCCTTGATTTATAATACCCTTACTA |
| 13 | Forward Primer | SEQ ID NO: 4 | ATGGAATCTTGCTGGTGCAT |
| | Probe | SEQ ID NO: 2 | CCCAATCTACAGCTGTCTTTACTGAATCAGGC |
| | Reverse Primer | SEQ ID NO: 5 | TGGCTTCTAACTGCATCTCT |
| | Forward Primer | SEQ ID NO: 23 | ACATGTTAGACGAAGAAGTTCAAAG |
| | Probe | SEQ ID NO: 19 | AGTGGTGATGCCTCCATATCACCAAGTG |
| | Reverse Primer | SEQ ID NO: 20 | AGCCCTTGATTTATAATACCCTTACT |
| 14 | Forward Primer | SEQ ID NO: 4 | ATGGAATCTTGCTGGTGCAT |
| | Probe | SEQ ID NO: 2 | CCCAATCTACAGCTGTCTTTACTGAATCAGGC |
| | Reverse Primer | SEQ ID NO: 5 | TGGCTTCTAACTGCATCTCT |
| | Forward Primer | SEQ ID NO: 24 | CATGTTAGACGAAGAAGTTCAAAGT |
| | Probe | SEQ ID NO: 16 | CTAGTGGTGATGCCTCCATATCACCAAGT |
| | Reverse Primer | SEQ ID NO: 25 | CTTTCACAGAAATTAGCCCTTGATT |
| 15 | Forward Primer | SEQ ID NO: 4 | ATGGAATCTTGCTGGTGCAT |
| | Probe | SEQ ID NO: 2 | CCCAATCTACAGCTGTCTTTACTGAATCAGGC |
| | Reverse Primer | SEQ ID NO: 5 | TGGCTTCTAACTGCATCTCT |
| | Forward Primer | SEQ ID NO: 26 | AGTAGTTTTGAATCTGTTCTAGCTTCT |
| | Probe | SEQ ID NO: 27 | TAGTGGTGATGCCTCCATATCACCAAGTG |
| | Reverse Primer | SEQ ID NO: 15 | AGCCCTTGATTTATAATACCCTTACTA |
| 16 | Forward Primer | SEQ ID NO: 4 | ATGGAATCTTGCTGGTGCAT |
| | Probe | SEQ ID NO: 2 | CCCAATCTACAGCTGTCTTTACTGAATCAGGC |
| | Reverse Primer | SEQ ID NO: 5 | TGGCTTCTAACTGCATCTCT |
| | Forward Primer | SEQ ID NO: 28 | TGAATCTGTTCTAGCTTCTAAGTCA |
| | Probe | SEQ ID NO: 29 | CACTTGGTGATATGGAGGCATCACCACT |
| | Reverse Primer | SEQ ID NO: 20 | AGCCCTTGATTTATAATACCCTTACT |
| 17 | Forward Primer | SEQ ID NO: 6 | GCTATATTGAAAAATATTGGTGGAGTCT |
| | Probe | SEQ ID NO: 2 | CCCAATCTACAGCTGTCTTTACTGAATCAGGC |
| | Reverse Primer | SEQ ID NO: 7 | TGGCTTCTAACTGCATCTCTT |
| | Forward Primer | SEQ ID NO: 13 | CAGAATATACCTCAGAACATTTTGACA |
| | Probe | SEQ ID NO: 14 | TCTAGTGGTGATGCCTCCATATCACCAAG |
| | Reverse Primer | SEQ ID NO: 15 | AGCCCTTGATTTATAATACCCTTACTA |
| 18 | Forward Primer | SEQ ID NO: 6 | GCTATATTGAAAAATATTGGTGGAGTCT |
| | Probe | SEQ ID NO: 2 | CCCAATCTACAGCTGTCTTTACTGAATCAGGC |
| | Reverse Primer | SEQ ID NO: 7 | TGGCTTCTAACTGCATCTCTT |
| | Forward Primer | SEQ ID NO: 13 | CAGAATATACCTCAGAACATTTTGACA |
| | Probe | SEQ ID NO: 16 | CTAGTGGTGATGCCTCCATATCACCAAGT |
| | Reverse Primer | SEQ ID NO: 17 | CTTTCACAGAAATTAGCCCTTGAT |
| 19 | Forward Primer | SEQ ID NO: 6 | GCTATATTGAAAAATATTGGTGGAGTCT |
| | Probe | SEQ ID NO: 2 | CCCAATCTACAGCTGTCTTTACTGAATCAGGC |
| | Reverse Primer | SEQ ID NO: 7 | TGGCTTCTAACTGCATCTCTT |
| | Forward Primer | SEQ ID NO: 18 | GAACATTTTGACATGTTAGACGAAGA |
| | Probe | SEQ ID NO: 19 | AGTGGTGATGCCTCCATATCACCAAGTG |
| | Reverse Primer | SEQ ID NO: 20 | AGCCCTTGATTTATAATACCCTTACT |
| 20 | Forward Primer | SEQ ID NO: 6 | GCTATATTGAAAAATATTGGTGGAGTCT |
| | Probe | SEQ ID NO: 2 | CCCAATCTACAGCTGTCTTTACTGAATCAGGC |
| | Reverse Primer | SEQ ID NO: 7 | TGGCTTCTAACTGCATCTCTT |
| | Forward Primer | SEQ ID NO: 21 | TGACATGTTAGACGAAGAAGTTCAA |
| | Probe | SEQ ID NO: 22 | TGCAATTTTAACTTCTAGTGGTGATGCCTCCA |
| | Reverse Primer | SEQ ID NO: 15 | AGCCCTTGATTTATAATACCCTTACTA |
| 21 | Forward Primer | SEQ ID NO: 6 | GCTATATTGAAAAATATTGGTGGAGTCT |
| | Probe | SEQ ID NO: 2 | CCCAATCTACAGCTGTCTTTACTGAATCAGGC |
| | Reverse Primer | SEQ ID NO: 7 | TGGCTTCTAACTGCATCTCTT |
| | Forward Primer | SEQ ID NO: 23 | ACATGTTAGACGAAGAAGTTCAAAG |
| | Probe | SEQ ID NO: 19 | AGTGGTGATGCCTCCATATCACCAAGTG |
| | Reverse Primer | SEQ ID NO: 20 | AGCCCTTGATTTATAATACCCTTACT |
| 22 | Forward Primer | SEQ ID NO: 6 | GCTATATTGAAAAATATTGGTGGAGTCT |
| | Probe | SEQ ID NO: 2 | CCCAATCTACAGCTGTCTTTACTGAATCAGGC |
| | Reverse Primer | SEQ ID NO: 7 | TGGCTTCTAACTGCATCTCTT |
| | Forward Primer | SEQ ID NO: 24 | CATGTTAGACGAAGAAGTTCAAAGT |
| | Probe | SEQ ID NO: 16 | CTAGTGGTGATGCCTCCATATCACCAAGT |
| | Reverse Primer | SEQ ID NO: 25 | CTTTCACAGAAATTAGCCCTTGATT |
| 23 | Forward Primer | SEQ ID NO: 6 | GCTATATTGAAAAATATTGGTGGAGTCT |
| | Probe | SEQ ID NO: 2 | CCCAATCTACAGCTGTCTTTACTGAATCAGGC |
| | Reverse Primer | SEQ ID NO: 7 | TGGCTTCTAACTGCATCTCTT |
| | Forward Primer | SEQ ID NO: 26 | AGTAGTTTTGAATCTGTTCTAGCTTCT |
| | Probe | SEQ ID NO: 27 | TAGTGGTGATGCCTCCATATCACCAAGTG |
| | Reverse Primer | SEQ ID NO: 15 | AGCCCTTGATTTATAATACCCTTACTA |
| 24 | Forward Primer | SEQ ID NO: 6 | GCTATATTGAAAAATATTGGTGGAGTCT |
| | Probe | SEQ ID NO: 2 | CCCAATCTACAGCTGTCTTTACTGAATCAGGC |
| | Reverse Primer | SEQ ID NO: 7 | TGGCTTCTAACTGCATCTCTT |
| | Forward Primer | SEQ ID NO: 28 | TGAATCTGTTCTAGCTTCTAAGTCA |
| | Probe | SEQ ID NO: 29 | CACTTGGTGATATGGAGGCATCACCACT |
| | Reverse Primer | SEQ ID NO: 20 | AGCCCTTGATTTATAATACCCTTACT |
| 25 | Forward Primer | SEQ ID NO: 8 | AATATTGGTGGAGTCTATCTAGATGTTG |
| | Probe | SEQ ID NO: 2 | CCCAATCTACAGCTGTCTTTACTGAATCAGGC |
| | Reverse Primer | SEQ ID NO: 9 | ATTATGGCTTCTAACTGCATCTCTT |
| | Forward Primer | SEQ ID NO: 13 | CAGAATATACCTCAGAACATTTTGACA |
| | Probe | SEQ ID NO: 14 | TCTAGTGGTGATGCCTCCATATCACCAAG |
| | Reverse Primer | SEQ ID NO: 15 | AGCCCTTGATTTATAATACCCTTACTA |
| 26 | Forward Primer | SEQ ID NO: 8 | AATATTGGTGGAGTCTATCTAGATGTTG |
| | Probe | SEQ ID NO: 2 | CCCAATCTACAGCTGTCTTTACTGAATCAGGC |
| | Reverse Primer | SEQ ID NO: 9 | ATTATGGCTTCTAACTGCATCTCTT |
| | Forward Primer | SEQ ID NO: 13 | CAGAATATACCTCAGAACATTTTGACA |
| | Probe | SEQ ID NO: 16 | CTAGTGGTGATGCCTCCATATCACCAAGT |
| | Reverse Primer | SEQ ID NO: 17 | CTTTCACAGAAATTAGCCCTTGAT |
| 27 | Forward Primer | SEQ ID NO: 8 | AATATTGGTGGAGTCTATCTAGATGTTG |
| | Probe | SEQ ID NO: 2 | CCCAATCTACAGCTGTCTTTACTGAATCAGGC |
| | Reverse Primer | SEQ ID NO: 9 | ATTATGGCTTCTAACTGCATCTCTT |
| | Forward Primer | SEQ ID NO: 18 | GAACATTTTGACATGTTAGACGAAGA |
| | Probe | SEQ ID NO: 19 | AGTGGTGATGCCTCCATATCACCAAGTG |
| | Reverse Primer | SEQ ID NO: 20 | AGCCCTTGATTTATAATACCCTTACT |
| 28 | Forward Primer | SEQ ID NO: 8 | AATATTGGTGGAGTCTATCTAGATGTTG |
| | Probe | SEQ ID NO: 2 | CCCAATCTACAGCTGTCTTTACTGAATCAGGC |
| | Reverse Primer | SEQ ID NO: 9 | ATTATGGCTTCTAACTGCATCTCTT |
| | Forward Primer | SEQ ID NO: 21 | TGACATGTTAGACGAAGAAGTTCAA |
| | Probe | SEQ ID NO: 22 | TGCAATTTTAACTTCTAGTGGTGATGCCTCCA |
| | Reverse Primer | SEQ ID NO: 15 | AGCCCTTGATTTATAATACCCTTACTA |
| 29 | Forward Primer | SEQ ID NO: 8 | AATATTGGTGGAGTCTATCTAGATGTTG |
| | Probe | SEQ ID NO: 2 | CCCAATCTACAGCTGTCTTTACTGAATCAGGC |
| | Reverse Primer | SEQ ID NO: 9 | ATTATGGCTTCTAACTGCATCTCTT |
| | Forward Primer | SEQ ID NO: 23 | ACATGTTAGACGAAGAAGTTCAAAG |
| | Probe | SEQ ID NO: 19 | AGTGGTGATGCCTCCATATCACCAAGTG |
| | Reverse Primer | SEQ ID NO: 20 | AGCCCTTGATTTATAATACCCTTACT |
| 30 | Forward Primer | SEQ ID NO: 8 | AATATTGGTGGAGTCTATCTAGATGTTG |
| | Probe | SEQ ID NO: 2 | CCCAATCTACAGCTGTCTTTACTGAATCAGGC |
| | Reverse Primer | SEQ ID NO: 9 | ATTATGGCTTCTAACTGCATCTCTT |
| | Forward Primer | SEQ ID NO: 24 | CATGTTAGACGAAGAAGTTCAAAGT |
| | Probe | SEQ ID NO: 16 | CTAGTGGTGATGCCTCCATATCACCAAGT |
| | Reverse Primer | SEQ ID NO: 25 | CTTTCACAGAAATTAGCCCTTGATT |
| 31 | Forward Primer | SEQ ID NO: 8 | AATATTGGTGGAGTCTATCTAGATGTTG |
| | Probe | SEQ ID NO: 2 | CCCAATCTACAGCTGTCTTTACTGAATCAGGC |
| | Reverse Primer | SEQ ID NO: 9 | ATTATGGCTTCTAACTGCATCTCTT |
| | Forward Primer | SEQ ID NO: 26 | AGTAGTTTTGAATCTGTTCTAGCTTCT |
| | Probe | SEQ ID NO: 27 | TAGTGGTGATGCCTCCATATCACCAAGTG |
| | Reverse Primer | SEQ ID NO: 15 | AGCCCTTGATTTATAATACCCTTACTA |
| 32 | Forward Primer | SEQ ID NO: 8 | AATATTGGTGGAGTCTATCTAGATGTTG |
| | Probe | SEQ ID NO: 2 | CCCAATCTACAGCTGTCTTTACTGAATCAGGC |
| | Reverse Primer | SEQ ID NO: 9 | ATTATGGCTTCTAACTGCATCTCTT |
| | Forward Primer | SEQ ID NO: 28 | TGAATCTGTTCTAGCTTCTAAGTCA |
| | Probe | SEQ ID NO: 29 | CACTTGGTGATATGGAGGCATCACCACT |
| | Reverse Primer | SEQ ID NO: 20 | AGCCCTTGATTTATAATACCCTTACT |
| 33 | Forward Primer | SEQ ID NO: 10 | TATTGGTGGAGTCTATCTAGATGTTG |
| | Probe | SEQ ID NO: 2 | CCCAATCTACAGCTGTCTTTACTGAATCAGGC |
| | Reverse Primer | SEQ ID NO: 11 | TATGGCTTCTAACTGCATCTCTT |
| | Forward Primer | SEQ ID NO: 13 | CAGAATATACCTCAGAACATTTTGACA |
| | Probe | SEQ ID NO: 14 | TCTAGTGGTGATGCCTCCATATCACCAAG |
| | Reverse Primer | SEQ ID NO: 15 | AGCCCTTGATTTATAATACCCTTACTA |
| 34 | Forward Primer | SEQ ID NO: 10 | TATTGGTGGAGTCTATCTAGATGTTG |
| | Probe | SEQ ID NO: 2 | CCCAATCTACAGCTGTCTTTACTGAATCAGGC |
| | Reverse Primer | SEQ ID NO: 11 | TATGGCTTCTAACTGCATCTCTT |
| | Forward Primer | SEQ ID NO: 13 | CAGAATATACCTCAGAACATTTTGACA |
| | Probe | SEQ ID NO: 16 | CTAGTGGTGATGCCTCCATATCACCAAGT |
| | Reverse Primer | SEQ ID NO: 17 | CTTTCACAGAAATTAGCCCTTGAT |
| 35 | Forward Primer | SEQ ID NO: 10 | TATTGGTGGAGTCTATCTAGATGTTG |
| | Probe | SEQ ID NO: 2 | CCCAATCTACAGCTGTCTTTACTGAATCAGGC |
| | Reverse Primer | SEQ ID NO: 11 | TATGGCTTCTAACTGCATCTCTT |
| | Forward Primer | SEQ ID NO: 18 | GAACATTTTGACATGTTAGACGAAGA |
| | Probe | SEQ ID NO: 19 | AGTGGTGATGCCTCCATATCACCAAGTG |
| | Reverse Primer | SEQ ID NO: 20 | AGCCCTTGATTTATAATACCCTTACT |
| 36 | Forward Primer | SEQ ID NO: 10 | TATTGGTGGAGTCTATCTAGATGTTG |
| | Probe | SEQ ID NO: 2 | CCCAATCTACAGCTGTCTTTACTGAATCAGGC |
| | Reverse Primer | SEQ ID NO: 11 | TATGGCTTCTAACTGCATCTCTT |
| | Forward Primer | SEQ ID NO: 21 | TGACATGTTAGACGAAGAAGTTCAA |
| | Probe | SEQ ID NO: 22 | TGCAATTTTAACTTCTAGTGGTGATGCCTCCA |
| | Reverse Primer | SEQ ID NO: 15 | AGCCCTTGATTTATAATACCCTTACTA |
| 37 | Forward Primer | SEQ ID NO: 10 | TATTGGTGGAGTCTATCTAGATGTTG |
| | Probe | SEQ ID NO: 2 | CCCAATCTACAGCTGTCTTTACTGAATCAGGC |
| | Reverse Primer | SEQ ID NO: 11 | TATGGCTTCTAACTGCATCTCTT |
| | Forward Primer | SEQ ID NO: 23 | ACATGTTAGACGAAGAAGTTCAAAG |
| | Probe | SEQ ID NO: 19 | AGTGGTGATGCCTCCATATCACCAAGTG |
| | Reverse Primer | SEQ ID NO: 20 | AGCCCTTGATTTATAATACCCTTACT |
| 38 | Forward Primer | SEQ ID NO: 10 | TATTGGTGGAGTCTATCTAGATGTTG |
| | Probe | SEQ ID NO: 2 | CCCAATCTACAGCTGTCTTTACTGAATCAGGC |
| | Reverse Primer | SEQ ID NO: 11 | TATGGCTTCTAACTGCATCTCTT |
| | Forward Primer | SEQ ID NO: 24 | CATGTTAGACGAAGAAGTTCAAAGT |
| | Probe | SEQ ID NO: 16 | CTAGTGGTGATGCCTCCATATCACCAAGT |
| | Reverse Primer | SEQ ID NO: 25 | CTTTCACAGAAATTAGCCCTTGATT |
| 39 | Forward Primer | SEQ ID NO: 10 | TATTGGTGGAGTCTATCTAGATGTTG |
| | Probe | SEQ ID NO: 2 | CCCAATCTACAGCTGTCTTTACTGAATCAGGC |
| | Reverse Primer | SEQ ID NO: 11 | TATGGCTTCTAACTGCATCTCTT |
| | Forward Primer | SEQ ID NO: 26 | AGTAGTTTTGAATCTGTTCTAGCTTCT |
| | Probe | SEQ ID NO: 27 | TAGTGGTGATGCCTCCATATCACCAAGTG |
| | Reverse Primer | SEQ ID NO: 15 | AGCCCTTGATTTATAATACCCTTACTA |
| 40 | Forward Primer | SEQ ID NO: 10 | TATTGGTGGAGTCTATCTAGATGTTG |
| | Probe | SEQ ID NO: 2 | CCCAATCTACAGCTGTCTTTACTGAATCAGGC |
| | Reverse Primer | SEQ ID NO: 11 | TATGGCTTCTAACTGCATCTCTT |
| | Forward Primer | SEQ ID NO: 28 | TGAATCTGTTCTAGCTTCTAAGTCA |
| | Probe | SEQ ID NO: 29 | CACTTGGTGATATGGAGGCATCACCACT |
| | Reverse Primer | SEQ ID NO: 20 | AGCCCTTGATTTATAATACCCTTACT |
| 41 | Forward Primer | SEQ ID NO: 12 | TGGTGGAGTCTATCTAGATGTTG |
| | Probe | SEQ ID NO: 2 | CCCAATCTACAGCTGTCTTTACTGAATCAGGC |
| | Reverse Primer | SEQ ID NO: 5 | TGGCTTCTAACTGCATCTCT |
| | Forward Primer | SEQ ID NO: 13 | CAGAATATACCTCAGAACATTTTGACA |
| | Probe | SEQ ID NO: 14 | TCTAGTGGTGATGCCTCCATATCACCAAG |
| | Reverse Primer | SEQ ID NO: 15 | AGCCCTTGATTTATAATACCCTTACTA |
| 42 | Forward Primer | SEQ ID NO: 12 | TGGTGGAGTCTATCTAGATGTTG |
| | Probe | SEQ ID NO: 2 | CCCAATCTACAGCTGTCTTTACTGAATCAGGC |
| | Reverse Primer | SEQ ID NO: 5 | TGGCTTCTAACTGCATCTCT |
| | Forward Primer | SEQ ID NO: 13 | CAGAATATACCTCAGAACATTTTGACA |
| | Probe | SEQ ID NO: 16 | CTAGTGGTGATGCCTCCATATCACCAAGT |
| | Reverse Primer | SEQ ID NO: 17 | CTTTCACAGAAATTAGCCCTTGAT |
| 43 | Forward Primer | SEQ ID NO: 12 | TGGTGGAGTCTATCTAGATGTTG |
| | Probe | SEQ ID NO: 2 | CCCAATCTACAGCTGTCTTTACTGAATCAGGC |
| | Reverse Primer | SEQ ID NO: 5 | TGGCTTCTAACTGCATCTCT |
| | Forward Primer | SEQ ID NO: 18 | GAACATTTTGACATGTTAGACGAAGA |
| | Probe | SEQ ID NO: 19 | AGTGGTGATGCCTCCATATCACCAAGTG |
| | Reverse Primer | SEQ ID NO: 20 | AGCCCTTGATTTATAATACCCTTACT |
| 44 | Forward Primer | SEQ ID NO: 12 | TGGTGGAGTCTATCTAGATGTTG |
| | Probe | SEQ ID NO: 2 | CCCAATCTACAGCTGTCTTTACTGAATCAGGC |
| | Reverse Primer | SEQ ID NO: 5 | TGGCTTCTAACTGCATCTCT |
| | Forward Primer | SEQ ID NO: 21 | TGACATGTTAGACGAAGAAGTTCAA |
| | Probe | SEQ ID NO: 22 | TGCAATTTTAACTTCTAGTGGTGATGCCTCCA |
| | Reverse Primer | SEQ ID NO: 15 | AGCCCTTGATTTATAATACCCTTACTA |
| 45 | Forward Primer | SEQ ID NO: 12 | TGGTGGAGTCTATCTAGATGTTG |
| | Probe | SEQ ID NO: 2 | CCCAATCTACAGCTGTCTTTACTGAATCAGGC |
| | Reverse Primer | SEQ ID NO: 5 | TGGCTTCTAACTGCATCTCT |
| | Forward Primer | SEQ ID NO: 23 | ACATGTTAGACGAAGAAGTTCAAAG |
| | Probe | SEQ ID NO: 19 | AGTGGTGATGCCTCCATATCACCAAGTG |
| | Reverse Primer | SEQ ID NO: 20 | AGCCCTTGATTTATAATACCCTTACT |
| 46 | Forward Primer | SEQ ID NO: 12 | TGGTGGAGTCTATCTAGATGTTG |
| | Probe | SEQ ID NO: 2 | CCCAATCTACAGCTGTCTTTACTGAATCAGGC |
| | Reverse Primer | SEQ ID NO: 5 | TGGCTTCTAACTGCATCTCT |
| | Forward Primer | SEQ ID NO: 24 | CATGTTAGACGAAGAAGTTCAAAGT |
| | Probe | SEQ ID NO: 16 | CTAGTGGTGATGCCTCCATATCACCAAGT |
| | Reverse Primer | SEQ ID NO: 25 | CTTTCACAGAAATTAGCCCTTGATT |
| 47 | Forward Primer | SEQ ID NO: 12 | TGGTGGAGTCTATCTAGATGTTG |
| | Probe | SEQ ID NO: 2 | CCCAATCTACAGCTGTCTTTACTGAATCAGGC |
| | Reverse Primer | SEQ ID NO: 5 | TGGCTTCTAACTGCATCTCT |
| | Forward Primer | SEQ ID NO: 26 | AGTAGTTTTGAATCTGTTCTAGCTTCT |
| | Probe | SEQ ID NO: 27 | TAGTGGTGATGCCTCCATATCACCAAGTG |
| | Reverse Primer | SEQ ID NO: 15 | AGCCCTTGATTTATAATACCCTTACTA |
| 48 | Forward Primer | SEQ ID NO: 12 | TGGTGGAGTCTATCTAGATGTTG |
| | Probe | SEQ ID NO: 2 | CCCAATCTACAGCTGTCTTTACTGAATCAGGC |
| | Reverse Primer | SEQ ID NO: 5 | TGGCTTCTAACTGCATCTCT |
| | Forward Primer | SEQ ID NO: 28 | TGAATCTGTTCTAGCTTCTAAGTCA |
| | Probe | SEQ ID NO: 29 | CACTTGGTGATATGGAGGCATCACCACT |
| | Reverse Primer | SEQ ID NO: 20 | AGCCCTTGATTTATAATACCCTTACT |
| 49 | Forward Primer | SEQ ID NO: 30 | ACTTTAGGTCCAATTATTAGTCAAGGTAA |
| | Probe | SEQ ID NO: 31 | AGCTCCCAAACTTTCACCAAAATTGTTCATTGTAG |
| | Reverse Primer | SEQ ID NO: 32 | CCAACCCTTAAATAACTTCCGATT |
| | Forward Primer | SEQ ID NO: 13 | CAGAATATACCTCAGAACATTTTGACA |
| | Probe | SEQ ID NO: 14 | TCTAGTGGTGATGCCTCCATATCACCAAG |
| | Reverse Primer | SEQ ID NO: 15 | AGCCCTTGATTTATAATACCCTTACTA |
| 50 | Forward Primer | SEQ ID NO: 30 | ACTTTAGGTCCAATTATTAGTCAAGGTAA |
| | Probe | SEQ ID NO: 31 | AGCTCCCAAACTTTCACCAAAATTGTTCATTGTAG |
| | Reverse Primer | SEQ ID NO: 32 | CCAACCCTTAAATAACTTCCGATT |
| | Forward Primer | SEQ ID NO: 13 | CAGAATATACCTCAGAACATTTTGACA |
| | Probe | SEQ ID NO: 16 | CTAGTGGTGATGCCTCCATATCACCAAGT |
| | Reverse Primer | SEQ ID NO: 17 | CTTTCACAGAAATTAGCCCTTGAT |
| 51 | Forward Primer | SEQ ID NO: 30 | ACTTTAGGTCCAATTATTAGTCAAGGTAA |
| | Probe | SEQ ID NO: 31 | AGCTCCCAAACTTTCACCAAAATTGTTCATTGTAG |
| | Reverse Primer | SEQ ID NO: 32 | CCAACCCTTAAATAACTTCCGATT |
| | Forward Primer | SEQ ID NO: 18 | GAACATTTTGACATGTTAGACGAAGA |
| | Probe | SEQ ID NO: 19 | AGTGGTGATGCCTCCATATCACCAAGTG |
| | Reverse Primer | SEQ ID NO: 20 | AGCCCTTGATTTATAATACCCTTACT |
| 52 | Forward Primer | SEQ ID NO: 30 | ACTTTAGGTCCAATTATTAGTCAAGGTAA |
| | Probe | SEQ ID NO: 31 | AGCTCCCAAACTTTCACCAAAATTGTTCATTGTAG |
| | Reverse Primer | SEQ ID NO: 32 | CCAACCCTTAAATAACTTCCGATT |
| | Forward Primer | SEQ ID NO: 21 | TGACATGTTAGACGAAGAAGTTCAA |
| | Probe | SEQ ID NO: 22 | TGCAATTTTAACTTCTAGTGGTGATGCCTCCA |
| | Reverse Primer | SEQ ID NO: 15 | AGCCCTTGATTTATAATACCCTTACTA |
| 53 | Forward Primer | SEQ ID NO: 30 | ACTTTAGGTCCAATTATTAGTCAAGGTAA |
| | Probe | SEQ ID NO: 31 | AGCTCCCAAACTTTCACCAAAATTGTTCATTGTAG |
| | Reverse Primer | SEQ ID NO: 32 | CCAACCCTTAAATAACTTCCGATT |
| | Forward Primer | SEQ ID NO: 23 | ACATGTTAGACGAAGAAGTTCAAAG |
| | Probe | SEQ ID NO: 19 | AGTGGTGATGCCTCCATATCACCAAGTG |
| | Reverse Primer | SEQ ID NO: 20 | AGCCCTTGATTTATAATACCCTTACT |
| 54 | Forward Primer | SEQ ID NO: 30 | ACTTTAGGTCCAATTATTAGTCAAGGTAA |
| | Probe | SEQ ID NO: 31 | AGCTCCCAAACTTTCACCAAAATTGTTCATTGTAG |
| | Reverse Primer | SEQ ID NO: 32 | CCAACCCTTAAATAACTTCCGATT |
| | Forward Primer | SEQ ID NO: 24 | CATGTTAGACGAAGAAGTTCAAAGT |
| | Probe | SEQ ID NO: 16 | CTAGTGGTGATGCCTCCATATCACCAAGT |
| | Reverse Primer | SEQ ID NO: 25 | CTTTCACAGAAATTAGCCCTTGATT |
| 55 | Forward Primer | SEQ ID NO: 30 | ACTTTAGGTCCAATTATTAGTCAAGGTAA |
| | Probe | SEQ ID NO: 31 | AGCTCCCAAACTTTCACCAAAATTGTTCATTGTAG |
| | Reverse Primer | SEQ ID NO: 32 | CCAACCCTTAAATAACTTCCGATT |
| | Forward Primer | SEQ ID NO: 26 | AGTAGTTTTGAATCTGTTCTAGCTTCT |
| | Probe | SEQ ID NO: 27 | TAGTGGTGATGCCTCCATATCACCAAGTG |
| | Reverse Primer | SEQ ID NO: 15 | AGCCCTTGATTTATAATACCCTTACTA |
| 56 | Forward Primer | SEQ ID NO: 30 | ACTTTAGGTCCAATTATTAGTCAAGGTAA |
| | Probe | SEQ ID NO: 31 | AGCTCCCAAACTTTCACCAAAATTGTTCATTGTAG |
| | Reverse Primer | SEQ ID NO: 32 | CCAACCCTTAAATAACTTCCGATT |
| | Forward Primer | SEQ ID NO: 28 | TGAATCTGTTCTAGCTTCTAAGTCA |
| | Probe | SEQ ID NO: 29 | CACTTGGTGATATGGAGGCATCACCACT |
| | Reverse Primer | SEQ ID NO: 20 | AGCCCTTGATTTATAATACCCTTACT |
| 57 | Forward Primer | SEQ ID NO: 30 | ACTTTAGGTCCAATTATTAGTCAAGGTAA |
| | Probe | SEQ ID NO: 31 | AGCTCCCAAACTTTCACCAAAATTGTTCATTGTAG |
| | Reverse Primer | SEQ ID NO: 32 | CCAACCCTTAAATAACTTCCGATT |
| | Forward Primer | SEQ ID NO: 37 | ATCTGCATTAAAAGAAATTGGTGGTA |
| | Probe | SEQ ID NO: 38 | TCCCAAAAATCCACTGTTACTGAACTAGGTTTCTC |
| | Reverse Primer | SEQ ID NO: 39 | CATGTCAAAATGTTCTGAGGTATATTCT |
| 58 | Forward Primer | SEQ ID NO: 30 | ACTTTAGGTCCAATTATTAGTCAAGGTAA |
| | Probe | SEQ ID NO: 31 | AGCTCCCAAACTTTCACCAAAATTGTTCATTGTAG |
| | Reverse Primer | SEQ ID NO: 33 | CAACCCTTAAATAACTTCCGATTTTTG |
| | Forward Primer | SEQ ID NO: 13 | CAGAATATACCTCAGAACATTTTGACA |
| | Probe | SEQ ID NO: 14 | TCTAGTGGTGATGCCTCCATATCACCAAG |
| | Reverse Primer | SEQ ID NO: 15 | AGCCCTTGATTTATAATACCCTTACTA |
| 59 | Forward Primer | SEQ ID NO: 30 | ACTTTAGGTCCAATTATTAGTCAAGGTAA |
| | Probe | SEQ ID NO: 31 | AGCTCCCAAACTTTCACCAAAATTGTTCATTGTAG |
| | Reverse Primer | SEQ ID NO: 33 | CAACCCTTAAATAACTTCCGATTTTTG |
| | Forward Primer | SEQ ID NO: 13 | CAGAATATACCTCAGAACATTTTGACA |
| | Probe | SEQ ID NO: 16 | CTAGTGGTGATGCCTCCATATCACCAAGT |
| | Reverse Primer | SEQ ID NO: 17 | CTTTCACAGAAATTAGCCCTTGAT |
| 60 | Forward Primer | SEQ ID NO: 30 | ACTTTAGGTCCAATTATTAGTCAAGGTAA |
| | Probe | SEQ ID NO: 31 | AGCTCCCAAACTTTCACCAAAATTGTTCATTGTAG |
| | Reverse Primer | SEQ ID NO: 33 | CAACCCTTAAATAACTTCCGATTTTTG |
| | Forward Primer | SEQ ID NO: 18 | GAACATTTTGACATGTTAGACGAAGA |
| | Probe | SEQ ID NO: 19 | AGTGGTGATGCCTCCATATCACCAAGTG |
| | Reverse Primer | SEQ ID NO: 20 | AGCCCTTGATTTATAATACCCTTACT |
| 61 | Forward Primer | SEQ ID NO: 30 | ACTTTAGGTCCAATTATTAGTCAAGGTAA |
| | Probe | SEQ ID NO: 31 | AGCTCCCAAACTTTCACCAAAATTGTTCATTGTAG |
| | Reverse Primer | SEQ ID NO: 33 | CAACCCTTAAATAACTTCCGATTTTTG |
| | Forward Primer | SEQ ID NO: 21 | TGACATGTTAGACGAAGAAGTTCAA |
| | Probe | SEQ ID NO: 22 | TGCAATTTTAACTTCTAGTGGTGATGCCTCCA |
| | Reverse Primer | SEQ ID NO: 15 | AGCCCTTGATTTATAATACCCTTACTA |
| 62 | Forward Primer | SEQ ID NO: 30 | ACTTTAGGTCCAATTATTAGTCAAGGTAA |
| | Probe | SEQ ID NO: 31 | AGCTCCCAAACTTTCACCAAAATTGTTCATTGTAG |
| | Reverse Primer | SEQ ID NO: 33 | CAACCCTTAAATAACTTCCGATTTTTG |
| | Forward Primer | SEQ ID NO: 23 | ACATGTTAGACGAAGAAGTTCAAAG |
| | Probe | SEQ ID NO: 19 | AGTGGTGATGCCTCCATATCACCAAGTG |
| | Reverse Primer | SEQ ID NO: 20 | AGCCCTTGATTTATAATACCCTTACT |
| 63 | Forward Primer | SEQ ID NO: 30 | ACTTTAGGTCCAATTATTAGTCAAGGTAA |
| | Probe | SEQ ID NO: 31 | AGCTCCCAAACTTTCACCAAAATTGTTCATTGTAG |
| | Reverse Primer | SEQ ID NO: 33 | CAACCCTTAAATAACTTCCGATTTTTG |
| | Forward Primer | SEQ ID NO: 24 | CATGTTAGACGAAGAAGTTCAAAGT |
| | Probe | SEQ ID NO: 16 | CTAGTGGTGATGCCTCCATATCACCAAGT |
| | Reverse Primer | SEQ ID NO: 25 | CTTTCACAGAAATTAGCCCTTGATT |
| 64 | Forward Primer | SEQ ID NO: 30 | ACTTTAGGTCCAATTATTAGTCAAGGTAA |
| | Probe | SEQ ID NO: 31 | AGCTCCCAAACTTTCACCAAAATTGTTCATTGTAG |
| | Reverse Primer | SEQ ID NO: 33 | CAACCCTTAAATAACTTCCGATTTTTG |
| | Forward Primer | SEQ ID NO: 26 | AGTAGTTTTGAATCTGTTCTAGCTTCT |
| | Probe | SEQ ID NO: 27 | TAGTGGTGATGCCTCCATATCACCAAGTG |
| | Reverse Primer | SEQ ID NO: 15 | AGCCCTTGATTTATAATACCCTTACTA |
| 65 | Forward Primer | SEQ ID NO: 30 | ACTTTAGGTCCAATTATTAGTCAAGGTAA |
| | Probe | SEQ ID NO: 31 | AGCTCCCAAACTTTCACCAAAATTGTTCATTGTAG |
| | Reverse Primer | SEQ ID NO: 33 | CAACCCTTAAATAACTTCCGATTTTTG |
| | Forward Primer | SEQ ID NO: 28 | TGAATCTGTTCTAGCTTCTAAGTCA |
| | Probe | SEQ ID NO: 29 | CACTTGGTGATATGGAGGCATCACCACT |
| | Reverse Primer | SEQ ID NO: 20 | AGCCCTTGATTTATAATACCCTTACT |
| 66 | Forward Primer | SEQ ID NO: 30 | ACTTTAGGTCCAATTATTAGTCAAGGTAA |
| | Probe | SEQ ID NO: 31 | AGCTCCCAAACTTTCACCAAAATTGTTCATTGTAG |
| | Reverse Primer | SEQ ID NO: 33 | CAACCCTTAAATAACTTCCGATTTTTG |
| | Forward Primer | SEQ ID NO: 37 | ATCTGCATTAAAAGAAATTGGTGGTA |
| | Probe | SEQ ID NO: 38 | TCCCAAAAATCCACTGTTACTGAACTAGGTTTCTC |
| | Reverse Primer | SEQ ID NO: 39 | CATGTCAAAATGTTCTGAGGTATATTCT |
| 67 | Forward Primer | SEQ ID NO: 30 | ACTTTAGGTCCAATTATTAGTCAAGGTAA |
| | Probe | SEQ ID NO: 31 | AGCTCCCAAACTTTCACCAAAATTGTTCATTGTAG |
| | Reverse Primer | SEQ ID NO: 34 | CTTCAGGATAAAATCCAACCCTTAAATA |
| | Forward Primer | SEQ ID NO: 13 | CAGAATATACCTCAGAACATTTTGACA |
| | Probe | SEQ ID NO: 14 | TCTAGTGGTGATGCCTCCATATCACCAAG |
| | Reverse Primer | SEQ ID NO: 15 | AGCCCTTGATTTATAATACCCTTACTA |
| 68 | Forward Primer | SEQ ID NO: 30 | ACTTTAGGTCCAATTATTAGTCAAGGTAA |
| | Probe | SEQ ID NO: 31 | AGCTCCCAAACTTTCACCAAAATTGTTCATTGTAG |
| | Reverse Primer | SEQ ID NO: 34 | CTTCAGGATAAAATCCAACCCTTAAATA |
| | Forward Primer | SEQ ID NO: 13 | CAGAATATACCTCAGAACATTTTGACA |
| | Probe | SEQ ID NO: 16 | CTAGTGGTGATGCCTCCATATCACCAAGT |
| | Reverse Primer | SEQ ID NO: 17 | CTTTCACAGAAATTAGCCCTTGAT |
| 69 | Forward Primer | SEQ ID NO: 30 | ACTTTAGGTCCAATTATTAGTCAAGGTAA |
| | Probe | SEQ ID NO: 31 | AGCTCCCAAACTTTCACCAAAATTGTTCATTGTAG |
| | Reverse Primer | SEQ ID NO: 34 | CTTCAGGATAAAATCCAACCCTTAAATA |
| | Forward Primer | SEQ ID NO: 18 | GAACATTTTGACATGTTAGACGAAGA |
| | Probe | SEQ ID NO: 19 | AGTGGTGATGCCTCCATATCACCAAGTG |
| | Reverse Primer | SEQ ID NO: 20 | AGCCCTTGATTTATAATACCCTTACT |
| 70 | Forward Primer | SEQ ID NO: 30 | ACTTTAGGTCCAATTATTAGTCAAGGTAA |
| | Probe | SEQ ID NO: 31 | AGCTCCCAAACTTTCACCAAAATTGTTCATTGTAG |
| | Reverse Primer | SEQ ID NO: 34 | CTTCAGGATAAAATCCAACCCTTAAATA |
| | Forward Primer | SEQ ID NO: 21 | TGACATGTTAGACGAAGAAGTTCAA |
| | Probe | SEQ ID NO: 22 | TGCAATTTTAACTTCTAGTGGTGATGCCTCCA |
| | Reverse Primer | SEQ ID NO: 15 | AGCCCTTGATTTATAATACCCTTACTA |
| 71 | Forward Primer | SEQ ID NO: 30 | ACTTTAGGTCCAATTATTAGTCAAGGTAA |
| | Probe | SEQ ID NO: 31 | AGCTCCCAAACTTTCACCAAAATTGTTCATTGTAG |
| | Reverse Primer | SEQ ID NO: 34 | CTTCAGGATAAAATCCAACCCTTAAATA |
| | Forward Primer | SEQ ID NO: 23 | ACATGTTAGACGAAGAAGTTCAAAG |
| | Probe | SEQ ID NO: 19 | AGTGGTGATGCCTCCATATCACCAAGTG |
| | Reverse Primer | SEQ ID NO: 20 | AGCCCTTGATTTATAATACCCTTACT |
| 72 | Forward Primer | SEQ ID NO: 30 | ACTTTAGGTCCAATTATTAGTCAAGGTAA |
| | Probe | SEQ ID NO: 31 | AGCTCCCAAACTTTCACCAAAATTGTTCATTGTAG |
| | Reverse Primer | SEQ ID NO: 34 | CTTCAGGATAAAATCCAACCCTTAAATA |
| | Forward Primer | SEQ ID NO: 24 | CATGTTAGACGAAGAAGTTCAAAGT |
| | Probe | SEQ ID NO: 16 | CTAGTGGTGATGCCTCCATATCACCAAGT |
| | Reverse Primer | SEQ ID NO: 25 | CTTTCACAGAAATTAGCCCTTGATT |
| 73 | Forward Primer | SEQ ID NO: 30 | ACTTTAGGTCCAATTATTAGTCAAGGTAA |
| | Probe | SEQ ID NO: 31 | AGCTCCCAAACTTTCACCAAAATTGTTCATTGTAG |
| | Reverse Primer | SEQ ID NO: 34 | CTTCAGGATAAAATCCAACCCTTAAATA |
| | Forward Primer | SEQ ID NO: 26 | AGTAGTTTTGAATCTGTTCTAGCTTCT |
| | Probe | SEQ ID NO: 27 | TAGTGGTGATGCCTCCATATCACCAAGTG |
| | Reverse Primer | SEQ ID NO: 15 | AGCCCTTGATTTATAATACCCTTACTA |
| 74 | Forward Primer | SEQ ID NO: 30 | ACTTTAGGTCCAATTATTAGTCAAGGTAA |
| | Probe | SEQ ID NO: 31 | AGCTCCCAAACTTTCACCAAAATTGTTCATTGTAG |
| | Reverse Primer | SEQ ID NO: 34 | CTTCAGGATAAAATCCAACCCTTAAATA |
| | Forward Primer | SEQ ID NO: 28 | TGAATCTGTTCTAGCTTCTAAGTCA |
| | Probe | SEQ ID NO: 29 | CACTTGGTGATATGGAGGCATCACCACT |
| | Reverse Primer | SEQ ID NO: 20 | AGCCCTTGATTTATAATACCCTTACT |
| 75 | Forward Primer | SEQ ID NO: 30 | ACTTTAGGTCCAATTATTAGTCAAGGTAA |
| | Probe | SEQ ID NO: 31 | AGCTCCCAAACTTTCACCAAAATTGTTCATTGTAG |
| | Reverse Primer | SEQ ID NO: 34 | CTTCAGGATAAAATCCAACCCTTAAATA |
| | Forward Primer | SEQ ID NO: 37 | ATCTGCATTAAAAGAAATTGGTGGTA |
| | Probe | SEQ ID NO: 38 | TCCCAAAAATCCACTGTTACTGAACTAGGTTTCTC |
| | Reverse Primer | SEQ ID NO: 39 | CATGTCAAAATGTTCTGAGGTATATTCT |
| 76 | Forward Primer | SEQ ID NO: 35 | TTTAGGTCCAATTATTAGTCAAGGTAATG |
| | Probe | SEQ ID NO: 31 | AGCTCCCAAACTTTCACCAAAATTGTTCATTGTAG |
| | Reverse Primer | SEQ ID NO: 32 | CCAACCCTTAAATAACTTCCGATT |
| | Forward Primer | SEQ ID NO: 13 | CAGAATATACCTCAGAACATTTTGACA |
| | Probe | SEQ ID NO: 14 | TCTAGTGGTGATGCCTCCATATCACCAAG |
| | Reverse Primer | SEQ ID NO: 15 | AGCCCTTGATTTATAATACCCTTACTA |
| 77 | Forward Primer | SEQ ID NO: 35 | TTTAGGTCCAATTATTAGTCAAGGTAATG |
| | Probe | SEQ ID NO: 31 | AGCTCCCAAACTTTCACCAAAATTGTTCATTGTAG |
| | Reverse Primer | SEQ ID NO: 32 | CCAACCCTTAAATAACTTCCGATT |
| | Forward Primer | SEQ ID NO: 13 | CAGAATATACCTCAGAACATTTTGACA |
| | Probe | SEQ ID NO: 16 | CTAGTGGTGATGCCTCCATATCACCAAGT |
| | Reverse Primer | SEQ ID NO: 17 | CTTTCACAGAAATTAGCCCTTGAT |
| 78 | Forward Primer | SEQ ID NO: 35 | TTTAGGTCCAATTATTAGTCAAGGTAATG |
| | Probe | SEQ ID NO: 31 | AGCTCCCAAACTTTCACCAAAATTGTTCATTGTAG |
| | Reverse Primer | SEQ ID NO: 32 | CCAACCCTTAAATAACTTCCGATT |
| | Forward Primer | SEQ ID NO: 18 | GAACATTTTGACATGTTAGACGAAGA |
| | Probe | SEQ ID NO: 19 | AGTGGTGATGCCTCCATATCACCAAGTG |
| | Reverse Primer | SEQ ID NO: 20 | AGCCCTTGATTTATAATACCCTTACT |
| 79 | Forward Primer | SEQ ID NO: 35 | TTTAGGTCCAATTATTAGTCAAGGTAATG |
| | Probe | SEQ ID NO: 31 | AGCTCCCAAACTTTCACCAAAATTGTTCATTGTAG |
| | Reverse Primer | SEQ ID NO: 32 | CCAACCCTTAAATAACTTCCGATT |
| | Forward Primer | SEQ ID NO: 21 | TGACATGTTAGACGAAGAAGTTCAA |
| | Probe | SEQ ID NO: 22 | TGCAATTTTAACTTCTAGTGGTGATGCCTCCA |
| | Reverse Primer | SEQ ID NO: 15 | AGCCCTTGATTTATAATACCCTTACTA |
| 80 | Forward Primer | SEQ ID NO: 35 | TTTAGGTCCAATTATTAGTCAAGGTAATG |
| | Probe | SEQ ID NO: 31 | AGCTCCCAAACTTTCACCAAAATTGTTCATTGTAG |
| | Reverse Primer | SEQ ID NO: 32 | CCAACCCTTAAATAACTTCCGATT |
| | Forward Primer | SEQ ID NO: 23 | ACATGTTAGACGAAGAAGTTCAAAG |
| | Probe | SEQ ID NO: 19 | AGTGGTGATGCCTCCATATCACCAAGTG |
| | Reverse Primer | SEQ ID NO: 20 | AGCCCTTGATTTATAATACCCTTACT |
| 81 | Forward Primer | SEQ ID NO: 35 | TTTAGGTCCAATTATTAGTCAAGGTAATG |
| | Probe | SEQ ID NO: 31 | AGCTCCCAAACTTTCACCAAAATTGTTCATTGTAG |
| | Reverse Primer | SEQ ID NO: 32 | CCAACCCTTAAATAACTTCCGATT |
| | Forward Primer | SEQ ID NO: 24 | CATGTTAGACGAAGAAGTTCAAAGT |
| | Probe | SEQ ID NO: 16 | CTAGTGGTGATGCCTCCATATCACCAAGT |
| | Reverse Primer | SEQ ID NO: 25 | CTTTCACAGAAATTAGCCCTTGATT |
| 82 | Forward Primer | SEQ ID NO: 35 | TTTAGGTCCAATTATTAGTCAAGGTAATG |
| | Probe | SEQ ID NO: 31 | AGCTCCCAAACTTTCACCAAAATTGTTCATTGTAG |
| | Reverse Primer | SEQ ID NO: 32 | CCAACCCTTAAATAACTTCCGATT |
| | Forward Primer | SEQ ID NO: 26 | AGTAGTTTTGAATCTGTTCTAGCTTCT |
| | Probe | SEQ ID NO: 27 | TAGTGGTGATGCCTCCATATCACCAAGTG |
| | Reverse Primer | SEQ ID NO: 15 | AGCCCTTGATTTATAATACCCTTACTA |
| 83 | Forward Primer | SEQ ID NO: 35 | TTTAGGTCCAATTATTAGTCAAGGTAATG |
| | Probe | SEQ ID NO: 31 | AGCTCCCAAACTTTCACCAAAATTGTTCATTGTAG |
| | Reverse Primer | SEQ ID NO: 32 | CCAACCCTTAAATAACTTCCGATT |
| | Forward Primer | SEQ ID NO: 28 | TGAATCTGTTCTAGCTTCTAAGTCA |
| | Probe | SEQ ID NO: 29 | CACTTGGTGATATGGAGGCATCACCACT |
| | Reverse Primer | SEQ ID NO: 20 | AGCCCTTGATTTATAATACCCTTACT |
| 84 | Forward Primer | SEQ ID NO: 35 | TTTAGGTCCAATTATTAGTCAAGGTAATG |
| | Probe | SEQ ID NO: 31 | AGCTCCCAAACTTTCACCAAAATTGTTCATTGTAG |
| | Reverse Primer | SEQ ID NO: 32 | CCAACCCTTAAATAACTTCCGATT |
| | Forward Primer | SEQ ID NO: 37 | ATCTGCATTAAAAGAAATTGGTGGTA |
| | Probe | SEQ ID NO: 38 | TCCCAAAAATCCACTGTTACTGAACTAGGTTTCTC |
| | Reverse Primer | SEQ ID NO: 39 | CATGTCAAAATGTTCTGAGGTATATTCT |
| 85 | Forward Primer | SEQ ID NO: 35 | TTTAGGTCCAATTATTAGTCAAGGTAATG |
| | Probe | SEQ ID NO: 31 | AGCTCCCAAACTTTCACCAAAATTGTTCATTGTAG |
| | Reverse Primer | SEQ ID NO: 33 | CAACCCTTAAATAACTTCCGATTTTTG |
| | Forward Primer | SEQ ID NO: 13 | CAGAATATACCTCAGAACATTTTGACA |
| | Probe | SEQ ID NO: 14 | TCTAGTGGTGATGCCTCCATATCACCAAG |
| | Reverse Primer | SEQ ID NO: 15 | AGCCCTTGATTTATAATACCCTTACTA |
| 86 | Forward Primer | SEQ ID NO: 35 | TTTAGGTCCAATTATTAGTCAAGGTAATG |
| | Probe | SEQ ID NO: 31 | AGCTCCCAAACTTTCACCAAAATTGTTCATTGTAG |
| | Reverse Primer | SEQ ID NO: 33 | CAACCCTTAAATAACTTCCGATTTTTG |
| | Forward Primer | SEQ ID NO: 13 | CAGAATATACCTCAGAACATTTTGACA |
| | Probe | SEQ ID NO: 16 | CTAGTGGTGATGCCTCCATATCACCAAGT |
| | Reverse Primer | SEQ ID NO: 17 | CTTTCACAGAAATTAGCCCTTGAT |
| 87 | Forward Primer | SEQ ID NO: 35 | TTTAGGTCCAATTATTAGTCAAGGTAATG |
| | Probe | SEQ ID NO: 31 | AGCTCCCAAACTTTCACCAAAATTGTTCATTGTAG |
| | Reverse Primer | SEQ ID NO: 33 | CAACCCTTAAATAACTTCCGATTTTTG |
| | Forward Primer | SEQ ID NO: 18 | GAACATTTTGACATGTTAGACGAAGA |
| | Probe | SEQ ID NO: 19 | AGTGGTGATGCCTCCATATCACCAAGTG |
| | Reverse Primer | SEQ ID NO: 20 | AGCCCTTGATTTATAATACCCTTACT |
| 88 | Forward Primer | SEQ ID NO: 35 | TTTAGGTCCAATTATTAGTCAAGGTAATG |
| | Probe | SEQ ID NO: 31 | AGCTCCCAAACTTTCACCAAAATTGTTCATTGTAG |
| | Reverse Primer | SEQ ID NO: 33 | CAACCCTTAAATAACTTCCGATTTTTG |
| | Forward Primer | SEQ ID NO: 21 | TGACATGTTAGACGAAGAAGTTCAA |
| | Probe | SEQ ID NO: 22 | TGCAATTTTAACTTCTAGTGGTGATGCCTCCA |
| | Reverse Primer | SEQ ID NO: 15 | AGCCCTTGATTTATAATACCCTTACTA |
| 89 | Forward Primer | SEQ ID NO: 35 | TTTAGGTCCAATTATTAGTCAAGGTAATG |
| | Probe | SEQ ID NO: 31 | AGCTCCCAAACTTTCACCAAAATTGTTCATTGTAG |
| | Reverse Primer | SEQ ID NO: 33 | CAACCCTTAAATAACTTCCGATTTTTG |
| | Forward Primer | SEQ ID NO: 23 | ACATGTTAGACGAAGAAGTTCAAAG |
| | Probe | SEQ ID NO: 19 | AGTGGTGATGCCTCCATATCACCAAGTG |
| | Reverse Primer | SEQ ID NO: 20 | AGCCCTTGATTTATAATACCCTTACT |
| 90 | Forward Primer | SEQ ID NO: 35 | TTTAGGTCCAATTATTAGTCAAGGTAATG |
| | Probe | SEQ ID NO: 31 | AGCTCCCAAACTTTCACCAAAATTGTTCATTGTAG |
| | Reverse Primer | SEQ ID NO: 33 | CAACCCTTAAATAACTTCCGATTTTTG |
| | Forward Primer | SEQ ID NO: 24 | CATGTTAGACGAAGAAGTTCAAAGT |
| | Probe | SEQ ID NO: 16 | CTAGTGGTGATGCCTCCATATCACCAAGT |
| | Reverse Primer | SEQ ID NO: 25 | CTTTCACAGAAATTAGCCCTTGATT |
| 91 | Forward Primer | SEQ ID NO: 35 | TTTAGGTCCAATTATTAGTCAAGGTAATG |
| | Probe | SEQ ID NO: 31 | AGCTCCCAAACTTTCACCAAAATTGTTCATTGTAG |
| | Reverse Primer | SEQ ID NO: 33 | CAACCCTTAAATAACTTCCGATTTTTG |
| | Forward Primer | SEQ ID NO: 26 | AGTAGTTTTGAATCTGTTCTAGCTTCT |
| | Probe | SEQ ID NO: 27 | TAGTGGTGATGCCTCCATATCACCAAGTG |
| | Reverse Primer | SEQ ID NO: 15 | AGCCCTTGATTTATAATACCCTTACTA |
| 92 | Forward Primer | SEQ ID NO: 35 | TTTAGGTCCAATTATTAGTCAAGGTAATG |
| | Probe | SEQ ID NO: 31 | AGCTCCCAAACTTTCACCAAAATTGTTCATTGTAG |
| | Reverse Primer | SEQ ID NO: 33 | CAACCCTTAAATAACTTCCGATTTTTG |
| | Forward Primer | SEQ ID NO: 28 | TGAATCTGTTCTAGCTTCTAAGTCA |
| | Probe | SEQ ID NO: 29 | CACTTGGTGATATGGAGGCATCACCACT |
| | Reverse Primer | SEQ ID NO: 20 | AGCCCTTGATTTATAATACCCTTACT |
| 93 | Forward Primer | SEQ ID NO: 35 | TTTAGGTCCAATTATTAGTCAAGGTAATG |
| | Probe | SEQ ID NO: 31 | AGCTCCCAAACTTTCACCAAAATTGTTCATTGTAG |
| | Reverse Primer | SEQ ID NO: 33 | CAACCCTTAAATAACTTCCGATTTTTG |
| | Forward Primer | SEQ ID NO: 37 | ATCTGCATTAAAAGAAATTGGTGGTA |
| | Probe | SEQ ID NO: 38 | TCCCAAAAATCCACTGTTACTGAACTAGGTTTCTC |
| | Reverse Primer | SEQ ID NO: 39 | CATGTCAAAATGTTCTGAGGTATATTCT |
| 94 | Forward Primer | SEQ ID NO: 35 | TTTAGGTCCAATTATTAGTCAAGGTAATG |
| | Probe | SEQ ID NO: 31 | AGCTCCCAAACTTTCACCAAAATTGTTCATTGTAG |
| | Reverse Primer | SEQ ID NO: 34 | CTTCAGGATAAAATCCAACCCTTAAATA |
| | Forward Primer | SEQ ID NO: 13 | CAGAATATACCTCAGAACATTTTGACA |
| | Probe | SEQ ID NO: 14 | TCTAGTGGTGATGCCTCCATATCACCAAG |
| | Reverse Primer | SEQ ID NO: 15 | AGCCCTTGATTTATAATACCCTTACTA |
| 95 | Forward Primer | SEQ ID NO: 35 | TTTAGGTCCAATTATTAGTCAAGGTAATG |
| | Probe | SEQ ID NO: 31 | AGCTCCCAAACTTTCACCAAAATTGTTCATTGTAG |
| | Reverse Primer | SEQ ID NO: 34 | CTTCAGGATAAAATCCAACCCTTAAATA |
| | Forward Primer | SEQ ID NO: 13 | CAGAATATACCTCAGAACATTTTGACA |
| | Probe | SEQ ID NO: 16 | CTAGTGGTGATGCCTCCATATCACCAAGT |
| | Reverse Primer | SEQ ID NO: 17 | CTTTCACAGAAATTAGCCCTTGAT |
| 96 | Forward Primer | SEQ ID NO: 35 | TTTAGGTCCAATTATTAGTCAAGGTAATG |
| | Probe | SEQ ID NO: 31 | AGCTCCCAAACTTTCACCAAAATTGTTCATTGTAG |
| | Reverse Primer | SEQ ID NO: 34 | CTTCAGGATAAAATCCAACCCTTAAATA |
| | Forward Primer | SEQ ID NO: 18 | GAACATTTTGACATGTTAGACGAAGA |
| | Probe | SEQ ID NO: 19 | AGTGGTGATGCCTCCATATCACCAAGTG |
| | Reverse Primer | SEQ ID NO: 20 | AGCCCTTGATTTATAATACCCTTACT |
| 97 | Forward Primer | SEQ ID NO: 35 | TTTAGGTCCAATTATTAGTCAAGGTAATG |
| | Probe | SEQ ID NO: 31 | AGCTCCCAAACTTTCACCAAAATTGTTCATTGTAG |
| | Reverse Primer | SEQ ID NO: 34 | CTTCAGGATAAAATCCAACCCTTAAATA |
| | Forward Primer | SEQ ID NO: 21 | TGACATGTTAGACGAAGAAGTTCAA |
| | Probe | SEQ ID NO: 22 | TGCAATTTTAACTTCTAGTGGTGATGCCTCCA |
| | Reverse Primer | SEQ ID NO: 15 | AGCCCTTGATTTATAATACCCTTACTA |
| 98 | Forward Primer | SEQ ID NO: 35 | TTTAGGTCCAATTATTAGTCAAGGTAATG |
| | Probe | SEQ ID NO: 31 | AGCTCCCAAACTTTCACCAAAATTGTTCATTGTAG |
| | Reverse Primer | SEQ ID NO: 34 | CTTCAGGATAAAATCCAACCCTTAAATA |
| | Forward Primer | SEQ ID NO: 23 | ACATGTTAGACGAAGAAGTTCAAAG |
| | Probe | SEQ ID NO: 19 | AGTGGTGATGCCTCCATATCACCAAGTG |
| | Reverse Primer | SEQ ID NO: 20 | AGCCCTTGATTTATAATACCCTTACT |
| 99 | Forward Primer | SEQ ID NO: 35 | TTTAGGTCCAATTATTAGTCAAGGTAATG |
| | Probe | SEQ ID NO: 31 | AGCTCCCAAACTTTCACCAAAATTGTTCATTGTAG |
| | Reverse Primer | SEQ ID NO: 34 | CTTCAGGATAAAATCCAACCCTTAAATA |
| | Forward Primer | SEQ ID NO: 24 | CATGTTAGACGAAGAAGTTCAAAGT |
| | Probe | SEQ ID NO: 16 | CTAGTGGTGATGCCTCCATATCACCAAGT |
| | Reverse Primer | SEQ ID NO: 25 | CTTTCACAGAAATTAGCCCTTGATT |
| 100 | Forward Primer | SEQ ID NO: 35 | TTTAGGTCCAATTATTAGTCAAGGTAATG |
| | Probe | SEQ ID NO: 31 | AGCTCCCAAACTTTCACCAAAATTGTTCATTGTAG |
| | Reverse Primer | SEQ ID NO: 34 | CTTCAGGATAAAATCCAACCCTTAAATA |
| | Forward Primer | SEQ ID NO: 26 | AGTAGTTTTGAATCTGTTCTAGCTTCT |
| | Probe | SEQ ID NO: 27 | TAGTGGTGATGCCTCCATATCACCAAGTG |
| | Reverse Primer | SEQ ID NO: 15 | AGCCCTTGATTTATAATACCCTTACTA |
| 101 | Forward Primer | SEQ ID NO: 35 | TTTAGGTCCAATTATTAGTCAAGGTAATG |
| | Probe | SEQ ID NO: 31 | AGCTCCCAAACTTTCACCAAAATTGTTCATTGTAG |
| | Reverse Primer | SEQ ID NO: 34 | CTTCAGGATAAAATCCAACCCTTAAATA |
| | Forward Primer | SEQ ID NO: 28 | TGAATCTGTTCTAGCTTCTAAGTCA |
| | Probe | SEQ ID NO: 29 | CACTTGGTGATATGGAGGCATCACCACT |
| | Reverse Primer | SEQ ID NO: 20 | AGCCCTTGATTTATAATACCCTTACT |
| 102 | Forward Primer | SEQ ID NO: 35 | TTTAGGTCCAATTATTAGTCAAGGTAATG |
| | Probe | SEQ ID NO: 31 | AGCTCCCAAACTTTCACCAAAATTGTTCATTGTAG |
| | Reverse Primer | SEQ ID NO: 34 | CTTCAGGATAAAATCCAACCCTTAAATA |
| | Forward Primer | SEQ ID NO: 37 | ATCTGCATTAAAAGAAATTGGTGGTA |
| | Probe | SEQ ID NO: 38 | TCCCAAAAATCCACTGTTACTGAACTAGGTTTCTC |
| | Reverse Primer | SEQ ID NO: 39 | CATGTCAAAATGTTCTGAGGTATATTCT |
| 103 | Forward Primer | SEQ ID NO: 36 | TTAGGTCCAATTATTAGTCAAGGTAATGA |
| | Probe | SEQ ID NO: 31 | AGCTCCCAAACTTTCACCAAAATTGTTCATTGTAG |
| | Reverse Primer | SEQ ID NO: 32 | CCAACCCTTAAATAACTTCCGATT |
| | Forward Primer | SEQ ID NO: 13 | CAGAATATACCTCAGAACATTTTGACA |
| | Probe | SEQ ID NO: 14 | TCTAGTGGTGATGCCTCCATATCACCAAG |
| | Reverse Primer | SEQ ID NO: 15 | AGCCCTTGATTTATAATACCCTTACTA |
| 104 | Forward Primer | SEQ ID NO: 36 | TTAGGTCCAATTATTAGTCAAGGTAATGA |
| | Probe | SEQ ID NO: 31 | AGCTCCCAAACTTTCACCAAAATTGTTCATTGTAG |
| | Reverse Primer | SEQ ID NO: 32 | CCAACCCTTAAATAACTTCCGATT |
| | Forward Primer | SEQ ID NO: 13 | CAGAATATACCTCAGAACATTTTGACA |
| | Probe | SEQ ID NO: 16 | CTAGTGGTGATGCCTCCATATCACCAAGT |
| | Reverse Primer | SEQ ID NO: 17 | CTTTCACAGAAATTAGCCCTTGAT |
| 105 | Forward Primer | SEQ ID NO: 36 | TTAGGTCCAATTATTAGTCAAGGTAATGA |
| | Probe | SEQ ID NO: 31 | AGCTCCCAAACTTTCACCAAAATTGTTCATTGTAG |
| | Reverse Primer | SEQ ID NO: 32 | CCAACCCTTAAATAACTTCCGATT |
| | Forward Primer | SEQ ID NO: 18 | GAACATTTTGACATGTTAGACGAAGA |
| | Probe | SEQ ID NO: 19 | AGTGGTGATGCCTCCATATCACCAAGTG |
| | Reverse Primer | SEQ ID NO: 20 | AGCCCTTGATTTATAATACCCTTACT |
| 106 | Forward Primer | SEQ ID NO: 36 | TTAGGTCCAATTATTAGTCAAGGTAATGA |
| | Probe | SEQ ID NO: 31 | AGCTCCCAAACTTTCACCAAAATTGTTCATTGTAG |
| | Reverse Primer | SEQ ID NO: 32 | CCAACCCTTAAATAACTTCCGATT |
| | Forward Primer | SEQ ID NO: 21 | TGACATGTTAGACGAAGAAGTTCAA |
| | Probe | SEQ ID NO: 22 | TGCAATTTTAACTTCTAGTGGTGATGCCTCCA |
| | Reverse Primer | SEQ ID NO: 15 | AGCCCTTGATTTATAATACCCTTACTA |
| 107 | Forward Primer | SEQ ID NO: 36 | TTAGGTCCAATTATTAGTCAAGGTAATGA |
| | Probe | SEQ ID NO: 31 | AGCTCCCAAACTTTCACCAAAATTGTTCATTGTAG |
| | Reverse Primer | SEQ ID NO: 32 | CCAACCCTTAAATAACTTCCGATT |
| | Forward Primer | SEQ ID NO: 23 | ACATGTTAGACGAAGAAGTTCAAAG |
| | Probe | SEQ ID NO: 19 | AGTGGTGATGCCTCCATATCACCAAGTG |
| | Reverse Primer | SEQ ID NO: 20 | AGCCCTTGATTTATAATACCCTTACT |
| 108 | Forward Primer | SEQ ID NO: 36 | TTAGGTCCAATTATTAGTCAAGGTAATGA |
| | Probe | SEQ ID NO: 31 | AGCTCCCAAACTTTCACCAAAATTGTTCATTGTAG |
| | Reverse Primer | SEQ ID NO: 32 | CCAACCCTTAAATAACTTCCGATT |
| | Forward Primer | SEQ ID NO: 24 | CATGTTAGACGAAGAAGTTCAAAGT |
| | Probe | SEQ ID NO: 16 | CTAGTGGTGATGCCTCCATATCACCAAGT |
| | Reverse Primer | SEQ ID NO: 25 | CTTTCACAGAAATTAGCCCTTGATT |
| 109 | Forward Primer | SEQ ID NO: 36 | TTAGGTCCAATTATTAGTCAAGGTAATGA |
| | Probe | SEQ ID NO: 31 | AGCTCCCAAACTTTCACCAAAATTGTTCATTGTAG |
| | Reverse Primer | SEQ ID NO: 32 | CCAACCCTTAAATAACTTCCGATT |
| | Forward Primer | SEQ ID NO: 26 | AGTAGTTTTGAATCTGTTCTAGCTTCT |
| | Probe | SEQ ID NO: 27 | TAGTGGTGATGCCTCCATATCACCAAGTG |
| | Reverse Primer | SEQ ID NO: 15 | AGCCCTTGATTTATAATACCCTTACTA |
| 110 | Forward Primer | SEQ ID NO: 36 | TTAGGTCCAATTATTAGTCAAGGTAATGA |
| | Probe | SEQ ID NO: 31 | AGCTCCCAAACTTTCACCAAAATTGTTCATTGTAG |
| | Reverse Primer | SEQ ID NO: 32 | CCAACCCTTAAATAACTTCCGATT |
| | Forward Primer | SEQ ID NO: 28 | TGAATCTGTTCTAGCTTCTAAGTCA |
| | Probe | SEQ ID NO: 29 | CACTTGGTGATATGGAGGCATCACCACT |
| | Reverse Primer | SEQ ID NO: 20 | AGCCCTTGATTTATAATACCCTTACT |
| 111 | Forward Primer | SEQ ID NO: 36 | TTAGGTCCAATTATTAGTCAAGGTAATGA |
| | Probe | SEQ ID NO: 31 | AGCTCCCAAACTTTCACCAAAATTGTTCATTGTAG |
| | Reverse Primer | SEQ ID NO: 32 | CCAACCCTTAAATAACTTCCGATT |
| | Forward Primer | SEQ ID NO: 37 | ATCTGCATTAAAAGAAATTGGTGGTA |
| | Probe | SEQ ID NO: 38 | TCCCAAAAATCCACTGTTACTGAACTAGGTTTCTC |
| | Reverse Primer | SEQ ID NO: 39 | CATGTCAAAATGTTCTGAGGTATATTCT |
| 112 | Forward Primer | SEQ ID NO: 36 | TTAGGTCCAATTATTAGTCAAGGTAATGA |
| | Probe | SEQ ID NO: 31 | AGCTCCCAAACTTTCACCAAAATTGTTCATTGTAG |
| | Reverse Primer | SEQ ID NO: 33 | CAACCCTTAAATAACTTCCGATTTTTG |
| | Forward Primer | SEQ ID NO: 13 | CAGAATATACCTCAGAACATTTTGACA |
| | Probe | SEQ ID NO: 14 | TCTAGTGGTGATGCCTCCATATCACCAAG |
| | Reverse Primer | SEQ ID NO: 15 | AGCCCTTGATTTATAATACCCTTACTA |
| 113 | Forward Primer | SEQ ID NO: 36 | TTAGGTCCAATTATTAGTCAAGGTAATGA |
| | Probe | SEQ ID NO: 31 | AGCTCCCAAACTTTCACCAAAATTGTTCATTGTAG |
| | Reverse Primer | SEQ ID NO: 33 | CAACCCTTAAATAACTTCCGATTTTTG |
| | Forward Primer | SEQ ID NO: 13 | CAGAATATACCTCAGAACATTTTGACA |
| | Probe | SEQ ID NO: 16 | CTAGTGGTGATGCCTCCATATCACCAAGT |
| | Reverse Primer | SEQ ID NO: 17 | CTTTCACAGAAATTAGCCCTTGAT |
| 114 | Forward Primer | SEQ ID NO: 36 | TTAGGTCCAATTATTAGTCAAGGTAATGA |
| | Probe | SEQ ID NO: 31 | AGCTCCCAAACTTTCACCAAAATTGTTCATTGTAG |
| | Reverse Primer | SEQ ID NO: 33 | CAACCCTTAAATAACTTCCGATTTTTG |
| | Forward Primer | SEQ ID NO: 18 | GAACATTTTGACATGTTAGACGAAGA |
| | Probe | SEQ ID NO: 19 | AGTGGTGATGCCTCCATATCACCAAGTG |
| | Reverse Primer | SEQ ID NO: 20 | AGCCCTTGATTTATAATACCCTTACT |
| 115 | Forward Primer | SEQ ID NO: 36 | TTAGGTCCAATTATTAGTCAAGGTAATGA |
| | Probe | SEQ ID NO: 31 | AGCTCCCAAACTTTCACCAAAATTGTTCATTGTAG |
| | Reverse Primer | SEQ ID NO: 33 | CAACCCTTAAATAACTTCCGATTTTTG |
| | Forward Primer | SEQ ID NO: 21 | TGACATGTTAGACGAAGAAGTTCAA |
| | Probe | SEQ ID NO: 22 | TGCAATTTTAACTTCTAGTGGTGATGCCTCCA |
| | Reverse Primer | SEQ ID NO: 15 | AGCCCTTGATTTATAATACCCTTACTA |
| 116 | Forward Primer | SEQ ID NO: 36 | TTAGGTCCAATTATTAGTCAAGGTAATGA |
| | Probe | SEQ ID NO: 31 | AGCTCCCAAACTTTCACCAAAATTGTTCATTGTAG |
| | Reverse Primer | SEQ ID NO: 33 | CAACCCTTAAATAACTTCCGATTTTTG |
| | Forward Primer | SEQ ID NO: 23 | ACATGTTAGACGAAGAAGTTCAAAG |
| | Probe | SEQ ID NO: 19 | AGTGGTGATGCCTCCATATCACCAAGTG |
| | Reverse Primer | SEQ ID NO: 20 | AGCCCTTGATTTATAATACCCTTACT |
| 117 | Forward Primer | SEQ ID NO: 36 | TTAGGTCCAATTATTAGTCAAGGTAATGA |
| | Probe | SEQ ID NO: 31 | AGCTCCCAAACTTTCACCAAAATTGTTCATTGTAG |
| | Reverse Primer | SEQ ID NO: 33 | CAACCCTTAAATAACTTCCGATTTTTG |
| | Forward Primer | SEQ ID NO: 24 | CATGTTAGACGAAGAAGTTCAAAGT |
| | Probe | SEQ ID NO: 16 | CTAGTGGTGATGCCTCCATATCACCAAGT |
| | Reverse Primer | SEQ ID NO: 25 | CTTTCACAGAAATTAGCCCTTGATT |
| 118 | Forward Primer | SEQ ID NO: 36 | TTAGGTCCAATTATTAGTCAAGGTAATGA |
| | Probe | SEQ ID NO: 31 | AGCTCCCAAACTTTCACCAAAATTGTTCATTGTAG |
| | Reverse Primer | SEQ ID NO: 33 | CAACCCTTAAATAACTTCCGATTTTTG |
| | Forward Primer | SEQ ID NO: 26 | AGTAGTTTTGAATCTGTTCTAGCTTCT |
| | Probe | SEQ ID NO: 27 | TAGTGGTGATGCCTCCATATCACCAAGTG |
| | Reverse Primer | SEQ ID NO: 15 | AGCCCTTGATTTATAATACCCTTACTA |
| 119 | Forward Primer | SEQ ID NO: 36 | TTAGGTCCAATTATTAGTCAAGGTAATGA |
| | Probe | SEQ ID NO: 31 | AGCTCCCAAACTTTCACCAAAATTGTTCATTGTAG |
| | Reverse Primer | SEQ ID NO: 33 | CAACCCTTAAATAACTTCCGATTTTTG |
| | Forward Primer | SEQ ID NO: 28 | TGAATCTGTTCTAGCTTCTAAGTCA |
| | Probe | SEQ ID NO: 29 | CACTTGGTGATATGGAGGCATCACCACT |
| | Reverse Primer | SEQ ID NO: 20 | AGCCCTTGATTTATAATACCCTTACT |
| 120 | Forward Primer | SEQ ID NO: 36 | TTAGGTCCAATTATTAGTCAAGGTAATGA |
| | Probe | SEQ ID NO: 31 | AGCTCCCAAACTTTCACCAAAATTGTTCATTGTAG |
| | Reverse Primer | SEQ ID NO: 33 | CAACCCTTAAATAACTTCCGATTTTTG |
| | Forward Primer | SEQ ID NO: 37 | ATCTGCATTAAAAGAAATTGGTGGTA |
| | Probe | SEQ ID NO: 38 | TCCCAAAAATCCACTGTTACTGAACTAGGTTTCTC |
| | Reverse Primer | SEQ ID NO: 39 | CATGTCAAAATGTTCTGAGGTATATTCT |
| 121 | Forward Primer | SEQ ID NO: 36 | TTAGGTCCAATTATTAGTCAAGGTAATGA |
| | Probe | SEQ ID NO: 31 | AGCTCCCAAACTTTCACCAAAATTGTTCATTGTAG |
| | Reverse Primer | SEQ ID NO: 34 | CTTCAGGATAAAATCCAACCCTTAAATA |
| | Forward Primer | SEQ ID NO: 13 | CAGAATATACCTCAGAACATTTTGACA |
| | Probe | SEQ ID NO: 14 | TCTAGTGGTGATGCCTCCATATCACCAAG |
| | Reverse Primer | SEQ ID NO: 15 | AGCCCTTGATTTATAATACCCTTACTA |
| 122 | Forward Primer | SEQ ID NO: 36 | TTAGGTCCAATTATTAGTCAAGGTAATGA |
| | Probe | SEQ ID NO: 31 | AGCTCCCAAACTTTCACCAAAATTGTTCATTGTAG |
| | Reverse Primer | SEQ ID NO: 34 | CTTCAGGATAAAATCCAACCCTTAAATA |
| | Forward Primer | SEQ ID NO: 13 | CAGAATATACCTCAGAACATTTTGACA |
| | Probe | SEQ ID NO: 16 | CTAGTGGTGATGCCTCCATATCACCAAGT |
| | Reverse Primer | SEQ ID NO: 17 | CTTTCACAGAAATTAGCCCTTGAT |
| 123 | Forward Primer | SEQ ID NO: 36 | TTAGGTCCAATTATTAGTCAAGGTAATGA |
| | Probe | SEQ ID NO: 31 | AGCTCCCAAACTTTCACCAAAATTGTTCATTGTAG |
| | Reverse Primer | SEQ ID NO: 34 | CTTCAGGATAAAATCCAACCCTTAAATA |
| | Forward Primer | SEQ ID NO: 18 | GAACATTTTGACATGTTAGACGAAGA |
| | Probe | SEQ ID NO: 19 | AGTGGTGATGCCTCCATATCACCAAGTG |
| | Reverse Primer | SEQ ID NO: 20 | AGCCCTTGATTTATAATACCCTTACT |
| 124 | Forward Primer | SEQ ID NO: 36 | TTAGGTCCAATTATTAGTCAAGGTAATGA |
| | Probe | SEQ ID NO: 31 | AGCTCCCAAACTTTCACCAAAATTGTTCATTGTAG |
| | Reverse Primer | SEQ ID NO: 34 | CTTCAGGATAAAATCCAACCCTTAAATA |
| | Forward Primer | SEQ ID NO: 21 | TGACATGTTAGACGAAGAAGTTCAA |
| | Probe | SEQ ID NO: 22 | TGCAATTTTAACTTCTAGTGGTGATGCCTCCA |
| | Reverse Primer | SEQ ID NO: 15 | AGCCCTTGATTTATAATACCCTTACTA |
| 125 | Forward Primer | SEQ ID NO: 36 | TTAGGTCCAATTATTAGTCAAGGTAATGA |
| | Probe | SEQ ID NO: 31 | AGCTCCCAAACTTTCACCAAAATTGTTCATTGTAG |
| | Reverse Primer | SEQ ID NO: 34 | CTTCAGGATAAAATCCAACCCTTAAATA |
| | Forward Primer | SEQ ID NO: 23 | ACATGTTAGACGAAGAAGTTCAAAG |
| | Probe | SEQ ID NO: 19 | AGTGGTGATGCCTCCATATCACCAAGTG |
| | Reverse Primer | SEQ ID NO: 20 | AGCCCTTGATTTATAATACCCTTACT |
| 126 | Forward Primer | SEQ ID NO: 36 | TTAGGTCCAATTATTAGTCAAGGTAATGA |
| | Probe | SEQ ID NO: 31 | AGCTCCCAAACTTTCACCAAAATTGTTCATTGTAG |
| | Reverse Primer | SEQ ID NO: 34 | CTTCAGGATAAAATCCAACCCTTAAATA |
| | Forward Primer | SEQ ID NO: 24 | CATGTTAGACGAAGAAGTTCAAAGT |
| | Probe | SEQ ID NO: 16 | CTAGTGGTGATGCCTCCATATCACCAAGT |
| | Reverse Primer | SEQ ID NO: 25 | CTTTCACAGAAATTAGCCCTTGATT |
| 127 | Forward Primer | SEQ ID NO: 36 | TTAGGTCCAATTATTAGTCAAGGTAATGA |
| | Probe | SEQ ID NO: 31 | AGCTCCCAAACTTTCACCAAAATTGTTCATTGTAG |
| | Reverse Primer | SEQ ID NO: 34 | CTTCAGGATAAAATCCAACCCTTAAATA |
| | Forward Primer | SEQ ID NO: 26 | AGTAGTTTTGAATCTGTTCTAGCTTCT |
| | Probe | SEQ ID NO: 27 | TAGTGGTGATGCCTCCATATCACCAAGTG |
| | Reverse Primer | SEQ ID NO: 15 | AGCCCTTGATTTATAATACCCTTACTA |
| 128 | Forward Primer | SEQ ID NO: 36 | TTAGGTCCAATTATTAGTCAAGGTAATGA |
| | Probe | SEQ ID NO: 31 | AGCTCCCAAACTTTCACCAAAATTGTTCATTGTAG |
| | Reverse Primer | SEQ ID NO: 34 | CTTCAGGATAAAATCCAACCCTTAAATA |
| | Forward Primer | SEQ ID NO: 28 | TGAATCTGTTCTAGCTTCTAAGTCA |
| | Probe | SEQ ID NO: 29 | CACTTGGTGATATGGAGGCATCACCACT |
| | Reverse Primer | SEQ ID NO: 20 | AGCCCTTGATTTATAATACCCTTACT |
| 129 | Forward Primer | SEQ ID NO: 36 | TTAGGTCCAATTATTAGTCAAGGTAATGA |
| | Probe | SEQ ID NO: 31 | AGCTCCCAAACTTTCACCAAAATTGTTCATTGTAG |
| | Reverse Primer | SEQ ID NO: 34 | CTTCAGGATAAAATCCAACCCTTAAATA |
| | Forward Primer | SEQ ID NO: 37 | ATCTGCATTAAAAGAAATTGGTGGTA |
| | Probe | SEQ ID NO: 38 | TCCCAAAAATCCACTGTTACTGAACTAGGTTTCTC |
| | Reverse Primer | SEQ ID NO: 39 | CATGTCAAAATGTTCTGAGGTATATTCT |
| 184 | Forward Primer | SEQ ID NO: 12 | TGGTGGAGTCTATCTAGATGTTG |
| | Probe | SEQ ID NO: 2 | CCCAATCTACAGCTGTCTTTACTGAATCAGGC |
| | Reverse Primer | SEQ ID NO: 5 | TGGCTTCTAACTGCATCTCT |
| | Forward Primer | SEQ ID NO: 28 | TGAATCTGTTCTAGCTTCTAAGTCA |
| | Probe | SEQ ID NO: 29 | CACTTGGTGATATGGAGGCATCACCACT |
| | Reverse Primer | SEQ ID NO: 20 | AGCCCTTGATTTATAATACCCTTACT |
| | Reverse Primer | SEQ ID NO: 138 | AGTCCTTGATTTATAATACCTTTACT |

TABLE 5
Optimized C. Difficile Primers and Probes for Detecting tcdA Gene (toxin A)

| Group | Oligo type | SEQ ID NO: | Sequence |
|---|---|---|---|
| 130 | Forward Primer | SEQ ID NO: 40 | TTAACCCAGCCATAGAGTCTGA |
| | Probe | SEQ ID NO: 41 | AGCGAGCTTCTGGCATAAAACCTACTTG |
| | Reverse Primer | SEQ ID NO: 42 | TCCTGGACCACTTAAACTTATTGT |
| 131 | Forward Primer | SEQ ID NO: 43 | TAACCCAGCCATAGAGTCTGA |
| | Probe | SEQ ID NO: 41 | AGCGAGCTTCTGGCATAAAACCTACTTG |
| | Reverse Primer | SEQ ID NO: 44 | CTCCTGGACCACTTAAACTTATTGT |
| 132 | Forward Primer | SEQ ID NO: 45 | AACCCAGCCATAGAGTCTGA |
| | Probe | SEQ ID NO: 46 | TGGAGCGAGCTTCTGGCATAAAACCTAC |
| | Reverse Primer | SEQ ID NO: 47 | ATAAGCTCCTGGACCACTTAAACT |
| 133 | Forward Primer | SEQ ID NO: 48 | CCTTAACCCAGCCATAGAGT |
| | Probe | SEQ ID NO: 49 | AGCGAGCTTCTGGCATAAAACCTACTT |
| | Reverse Primer | SEQ ID NO: 50 | GCTCCTGGACCACTTAAACT |
| 134 | Forward Primer | SEQ ID NO: 51 | CAACACCTTAACCCAGCCAT |
| | Probe | SEQ ID NO: 41 | AGCGAGCTTCTGGCATAAAACCTACTTG |
| | Reverse Primer | SEQ ID NO: 42 | TCCTGGACCACTTAAACTTATTGT |
| 135 | Forward Primer | SEQ ID NO: 48 | CCTTAACCCAGCCATAGAGT |
| | Probe | SEQ ID NO: 46 | TGGAGCGAGCTTCTGGCATAAAACCTAC |
| | Reverse Primer | SEQ ID NO: 52 | ATAAGCTCCTGGACCACTTAAAC |
| 136 | Forward Primer | SEQ ID NO: 53 | CAACACCTTAACCCAGCCATAG |
| | Probe | SEQ ID NO: 46 | TGGAGCGAGCTTCTGGCATAAAACCTAC |
| | Reverse Primer | SEQ ID NO: 54 | AGCTCCTGGACCACTTAAACT |
| 137 | Forward Primer | SEQ ID NO: 55 | AATTTTTAAACCAACACCTTAACCCA |
| | Probe | SEQ ID NO: 56 | AGCGAGCTTCTGGCATAAAACCTACTTGT |
| | Reverse Primer | SEQ ID NO: 42 | TCCTGGACCACTTAAACTTATTGT |
| 138 | Forward Primer | SEQ ID NO: 55 | AATTTTTAAACCAACACCTTAACCCA |
| | Probe | SEQ ID NO: 56 | AGCGAGCTTCTGGCATAAAACCTACTTGT |
| | Reverse Primer | SEQ ID NO: 57 | TAAGCTCCTGGACCACTTAAACT |

TABLE 6
Optimized C. Difficile Primers and Probes for
Detecting cdtB Gene (binary toxin)

| | | SEQ ID NO: | |
|---|---|---|---|
| Group | Oligo type | NO: | Sequence |
| 139 | Forward Primer | SEQ ID NO: 58 | CCATTTATCCCAAATAACAATTTCTTTGAC |
| | Probe | SEQ ID NO: 59 | CCAAATCTTCGTCTTCCCAATCAGACATCAACT |
| | Reverse Primer | SEQ ID NO: 60 | AGTCCTTAATAGTATATCCATTTCGTTCA |
| 140 | Forward Primer | SEQ ID NO: 58 | CCATTTATCCCAAATAACAATTTCTTTGAC |
| | Probe | SEQ ID NO: 61 | AAATCTTCGTCTTCCCAATCAGACATCAACTTTGG |
| | Reverse Primer | SEQ ID NO: 60 | AGTCCTTAATAGTATATCCATTTCGTTCA |
| 141 | Forward Primer | SEQ ID NO: 58 | CCATTTATCCCAAATAACAATTTCTTTGAC |
| | Probe | SEQ ID NO: 59 | CCAAATCTTCGTCTTCCCAATCAGACATCAACT |
| | Reverse Primer | SEQ ID NO: 62 | AAGTCCTTAATAGTATATCCATTTCGTTCA |
| 142 | Forward Primer | SEQ ID NO: 63 | AGACGAAGATTTGGATACAGATAATGA |
| | Probe | SEQ ID NO: 64 | TTCTTATAGCCTTGTTCTGCAAAACTATCTTCCCA |
| | Reverse Primer | SEQ ID NO: 65 | TGGATCTCCAGCAGTATTTGA |
| 143 | Forward Primer | SEQ ID NO: 66 | AAGACGAAGATTTGGATACAGATAATGA |
| | Probe | SEQ ID NO: 64 | TTCTTATAGCCTTGTTCTGCAAAACTATCTTCCCA |
| | Reverse Primer | SEQ ID NO: 67 | TATGGATCTCCAGCAGTATTTGA |
| 144 | Forward Primer | SEQ ID NO: 68 | GATGATCCATTTATCCCAAATAACAATTTC |
| | Probe | SEQ ID NO: 69 | CCAAATCTTCGTCTTCCCAATCAGACATCAACTT |
| | Reverse Primer | SEQ ID NO: 60 | AGTCCTTAATAGTATATCCATTTCGTTCA |
| 145 | Forward Primer | SEQ ID NO: 68 | GATGATCCATTTATCCCAAATAACAATTTC |
| | Probe | SEQ ID NO: 59 | CCAAATCTTCGTCTTCCCAATCAGACATCAACT |
| | Reverse Primer | SEQ ID NO: 60 | AGTCCTTAATAGTATATCCATTTCGTTCA |

TABLE 7
Primers and Probe for Internal Control

| Group | Forward Primer | Probe | Reverse Primer |
|---|---|---|---|
| 146 | SEQ ID NO: 70 | SEQ ID NO: 71 | SEQ ID NO: 72 |
| | CAGACCGATAGCA | TGCTGCTCTGACA | TCCCTTGGTGGTG |
| | TAGCACTTAAA | ACTATACTCTCAG | AATCAAT |
| | | GATACA | |

TABLE 8
Primers and Probes for Detecting Geobacillus stearothermophilus (Process Control)

| Group | Forward Primer | Probe | Reverse Primer |
|---|---|---|---|
| 147 | SEQ ID NO: 88 | SEQ ID NO: 102 | SEQ ID NO: 108 |
| | ATTGTAGGTCTAGATCGGGAAG | ATCTCCATTCGTTGAGATCAATTTGGCAC | CTTTCAGTCGGTAAGGCAAG |
| 148 | SEQ ID NO: 84 | SEQ ID NO: 94 | SEQ ID NO: 96 |
| | AAATGCAGATGTTGTAATTGTAGGT | CAAGACAGGCTACAGCCAATTTTTCATACG | TTTGGCACCATTTCGTACAG |
| 149 | SEQ ID NO: 83 | SEQ ID NO: 94 | SEQ ID NO: 96 |
| | GAAAATGCAGATGTTGTAATTGTAGG | CAAGACAGGCTACAGCCAATTTTTCATACG | TTTGGCACCATTTCGTACAG |
| 150 | SEQ ID NO: 85 | SEQ ID NO: 94 | SEQ ID NO: 96 |
| | ATGCAGATGTTGTAATTGTAGGTC | CAAGACAGGCTACAGCCAATTTTTCATACG | TTTGGCACCATTTCGTACAG |
| 151 | SEQ ID NO: 75 | SEQ ID NO: 78 | SEQ ID NO: 80 |
| | CTGTTTCAGCGTTTAGGCAT | TCCTTTCATTACTTAACACACTTATGTCCCCT | CCATTATATTTTCTCATCGAA |
| | | | CCTGT |
| 152 | SEQ ID NO: 90 | SEQ ID NO: 98 | SEQ ID NO: 104 |
| | GATCGGGAAGTTACGTATGAAAAA | TTGAGATCAATTTGGCACCATTTCGTACAG | GTAAGGCAAGATCTCCATTCG |
| 153 | SEQ ID NO: 88 | SEQ ID NO: 98 | SEQ ID NO: 104 |
| | ATTGTAGGTCTAGATCGGGAAG | TTGAGATCAATTTGGCACCATTTCGTACAG | GTAAGGCAAGATCTCCATTCG |
| 154 | SEQ ID NO: 76 | SEQ ID NO: 81 | SEQ ID NO: 82 |
| | GAAACAGGGGACATAAGTGTG | CCCGATACATTGTTCCGTCCAAATCAA | CATCAATCCGCTCCGTTC |
| 155 | SEQ ID NO: 89 | SEQ ID NO: 102 | SEQ ID NO: 108 |
| | TCTAGATCGGGAAGTTACGTATG | ATCTCCATTCGTTGAGATCAATTTGGCAC | CTTTCAGTCGGTAAGGCAAG |
| 156 | SEQ ID NO: 93 | SEQ ID NO: 95 | SEQ ID NO: 109 |
| | TGTAGCCTGTCTTGCTGT | ACGAAATGGTGCCAAATTGATCTCAACG | CCGGCATAAATCCCCTTTC |
| 157 | SEQ ID NO: 87 | SEQ ID NO: 100 | SEQ ID NO: 107 |
| | TAATTGTAGGTCTAGATCGGGAAG | CTCCATTCGTTGAGATCAATTTGGCACC | TTCAGTCGGTAAGGCAAGAT |
| 158 | SEQ ID NO: 87 | SEQ ID NO: 97 | SEQ ID NO: 103 |
| | TAATTGTAGGTCTAGATCGGGAAG | TGAGATCAATTTGGCACCATTTCGTACAG | TAAGGCAAGATCTCCATTCGT |
| 159 | SEQ ID NO: 88 | SEQ IN NO: 97 | SEQ ID NO: 103 |
| | ATTGTAGGTCTAGATCGGGAAG | TGAGATCAATTTGGCACCATTTCGTACAG | TAAGGCAAGATCTCCATTCGT |
| 160 | SEQ ID NO: 92 | SEQ ID NO: 101 | SEQ ID NO: 106 |
| | AGTTACGTATGAAAAATTGGCTGT | TCTCCATTCGTTGAGATCAATTTGGCAC | TTCAGTCGGTAAGGCAAGA |
| 161 | SEQ ID NO: 88 | SEQ ID NO: 101 | SEQ ID NO: 106 |
| | ATTGTAGGTCTAGATCGGGAAG | TCTCCATTCGTTGAGATCAATTTGGCAC | TTCAGTCGGTAAGGCAAGA |
| 162 | SEQ ID NO: 87 | SEQ ID NO: 102 | SEQ ID NO: 108 |
| | TAATTGTAGGTCTAGATCGGGAAG | ATCTCCATTCGTTGAGATCAATTTGGCAC | CTTTCAGTCGGTAAGGCAAG |
| 163 | SEQ ID NO: 74 | SEQ ID NO: 77 | SEQ ID NO: 79 |
| | CAAACGAATTAGGGCCTGTT | AACACACTTATGTCCCCTGTTTCATCTCAT | CGAACCTGTTCCTTTCATTACTT |
| 164 | SEQ ID NO: 86 | SEQ ID NO: 94 | SEQ ID NO: 96 |
| | TGCAGATGTTGTAATTGTAGGTC | CAAGACAGGCTACAGCCAATTTTTCATACG | TTTGGCACCATTTCGTACAG |
| 165 | SEQ ID NO: 91 | SEQ ID NO: 99 | SEQ ID NO: 105 |
| | AAGTTACGTATGAAAAATTGGCTGTA | ATTCGTTGAGATCAATTTGGCACCATTTCG | TCGGTAAGGCAAGATCTCC |

TABLE 9
Primers and Probes for Detecting Corynebacterium glutamicum (Process Control)

| Group No. | Forward Primer | Probe | Reverse Primer |
|---|---|---|---|
| 166 | SEQ ID NO: 127 | SEQ ID NO: 130 | SEQ ID NO: 120 |
| | GCCAAATTGTGCAATCGT | TTTCACAACCTGAGAATGTCACAACACAT | CTTAAGAAGCTCGCCGTTAC |
| 167 | SEQ ID NO: 128 | SEQ ID NO: 134 | SEQ ID NO: 114 |
| | GCCAAATTGTGCAATCGTT | TCACAACCTGAGAATGTCACAACACATTA | TACGAATTGGGCCGAAAAAG |
| 168 | SEQ ID NO: 128 | SEQ ID NO: 133 | SEQ ID NO: 122 |
| | GCCAAATTGTGCAATCGTT | TTCACAACCTGAGAATGTCACAACACATTA | ACTTAAGAAGCTCGCCGTTA |
| 169 | SEQ ID NO: 128 | SEQ ID NO: 133 | SEQ ID NO: 112 |
| | GCCAAATTGTGCAATCGTT | TTCACAACCTGAGAATGTCACAACACATTA | TTGGGCCGAAAAAGAATCG |
| 170 | SEQ ID NO: 128 | SEQ ID NO: 133 | SEQ ID NO: 113 |
| | GCCAAATTGTGCAATCGTT | TTCACAACCTGAGAATGTCACAACACATTA | GAATTGGGCCGAAAAAGAATC |
| 171 | SEQ ID NO: 128 | SEQ ID NO: 132 | SEQ ID NO: 115 |
| | GCCAAATTGTGCAATCGTT | TTCACAACCTGAGAATGTCACAACACAT | TTACGAATTGGGCCGAAAA |
| 172 | SEQ ID NO: 128 | SEQ ID NO: 134 | SEQ ID NO: 117 |
| | GCCAAATTGTGCAATCGTT | TCACAACCTGAGAATGTCACAACACATTA | TTAAGAAGCTCGCCGTTAC |
| 173 | SEQ ID NO: 128 | SEQ ID NO: 133 | SEQ ID NO: 117 |
| | GCCAAATTGTGCAATCGTT | TTCACAACCTGAGAATGTCACAACACATTA | TTAAGAAGCTCGCCGTTAC |
| 174 | SEQ ID NO: 127 | SEQ ID NO: 130 | SEQ ID NO: 119 |
| | GCCAAATTGTGCAATCGT | TTTCACAACCTGAGAATGTCACAACACAT | CTTAAGAAGCTCGCCGTTA |
| 175 | SEQ ID NO: 127 | SEQ ID NO: 129 | SEQ ID NO: 111 |
| | GCCAAATTGTGCAATCGT | TTTCACAACCTGAGAATGTCACAACACA | TGGGCCGAAAAAGAATCG |
| 176 | SEQ ID NO: 127 | SEQ ID NO: 130 | SEQ ID NO: 110 |
| | GCCAAATTGTGCAATCGT | TTTCACAACCTGAGAATGTCACAACACAT | GGCCGAAAAAGAATCGGA |
| 177 | SEQ ID NO: 127 | SEQ ID NO: 130 | SEQ ID NO: 118 |
| | GCCAAATTGTGCAATCGT | TTTCACAACCTGAGAATGTCACAACACAT | CTTAAGAAGCTCGCCGTT |
| 178 | SEQ ID NO: 128 | SEQ ID NO: 133 | SEQ ID NO: 116 |
| | GCCAAATTGTGCAATCGTT | TTCACAACCTGAGAATGTCACAACACATTA | TAAGAAGCTCGCCGTTAC |
| 179 | SEQ ID NO: 127 | SEQ ID NO: 129 | SEQ ID NO: 116 |
| | GCCAAATTGTGCAATCGT | TTTCACAACCTGAGAATGTCACAACACA | TAAGAAGCTCGCCGTTAC |
| 180 | SEQ ID NO: 136 | SEQ ID NO: 123 | SEQ ID NO: 126 |
| | AACCTGAGAATGTCACAACAC | CACTTAAGAAGCTCGCCGTTACGAATTG | GGCAAGAGCCTTTCTTGT |
| 181 | SEQ ID NO: 135 | SEQ ID NO: 121 | SEQ ID NO: 125 |
| | CAACCTGAGAATGTCACAACA | CTTAAGAAGCTCGCCGTTACGAATTG | CAAGAGCCTTTCTTGTCCA |
| 182 | SEQ ID NO: 131 | SEQ ID NO: 123 | SEQ ID NO: 126 |
| | TTCACAACCTGAGAATGTCAC | CACTTAAGAAGCTCGCCGTTACGAATTG | GGCAAGAGCCTTTCTTGT |
| 183 | SEQ ID NO: 137 | SEQ ID NO: 124 | SEQ ID NO: 126 |
| | CCTGAGAATGTCACAACACATTA | CCACTTAAGAAGCTCGCCGTTACGAAT | GGCAAGAGCCTTTCTTGT |

A PCR primer set for amplifying C. Difficile sequences comprises at least one of the following sets of primer sequences: (1) SEQ ID NOS: 1 and 3; (2) SEQ ID NOS: 13 and 15; (3) SEQ ID NOS: 13 and 17; (4) SEQ ID NOS: 18 and 20; (5) SEQ ID NOS: 21 and 15; (6) SEQ ID NOS: 23 and 20; (7) SEQ ID NOS: 24 and 25; (8) SEQ ID NOS: 26 and 15; (9) SEQ ID NOS: 28 and 20; (10) SEQ ID NOS: 4 and 5; (11) SEQ ID NOS: 6 and 7; (12) SEQ ID NOS: 8 and 9; (13) SEQ ID NOS: 10 and 11; (14) SEQ ID NOS: 12 and 5; (15) SEQ ID NOS: 30 and 32; (16) SEQ ID NOS: 37 and 39; (17) SEQ ID NOS: 30 and 33; (18) SEQ ID NOS: 30 and 34; (19) SEQ ID NOS: 35 and 32; (20) SEQ ID NOS: 35 and 33; (21) SEQ ID NOS: 35 and 34; (22) SEQ ID NOS: 36 and 32; (23) SEQ ID NOS: 36 and 33; (24) SEQ ID NOS: 36 and 34; (25) SEQ ID NOS: 40 and 42; (26) SEQ ID NOS: 43 and 44; (27) SEQ ID NOS: 45 and 47; (28) SEQ ID NOS: 48 and 50; (29) SEQ ID NOS: 51 and 42; (30) SEQ ID NOS: 48 and 52; (31) SEQ ID NOS: 53 and 54; (32) SEQ ID NOS: 55 and 42; (33) SEQ ID NOS: 55 and 57; (34) SEQ ID NOS: 58 and 60; (35) SEQ ID NOS: 58 and 62; (36) SEQ ID NOS: 63 and 65; (37) SEQ ID NOS: 66 and 67; (38) SEQ ID NOS: 68 and 60; and (39) SEQ ID NOS: 28 and 138.

Any set of primers can be used simultaneously in a multiplex reaction with one or more other primer sets, so that multiple amplicons are amplified simultaneously.

A probe for binding to a C. Difficile sequence comprises at least one of the following probe sequences: SEQ ID NOS: 2, 14, 16, 19, 22, 27, 29, 31, 38, 41, 46, 49, 56, 59, 61, 64, and 69.

A PCR primer set for amplifying sequences encoding C. Difficile tcdB gene (toxin B) comprises at least one of the following sets of primer sequences: (1) SEQ ID NOS: 1 and 3; (2) SEQ ID NOS: 13 and 15; (3) SEQ ID NOS: 13 and 17; (4) SEQ ID NOS: 18 and 20; (5) SEQ ID NOS: 21 and 15; (6) SEQ ID NOS: 23 and 20; (7) SEQ ID NOS: 24 and 25; (8) SEQ ID NOS: 26 and 15; (9) SEQ ID NOS: 28 and 20; (10) SEQ ID NOS: 4 and 5; (11) SEQ ID NOS: 6 and 7; (12) SEQ ID NOS: 8 and 9; (13) SEQ ID NOS: 10 and 11; (14) SEQ ID NOS: 12 and 5; (15) SEQ ID NOS: 30 and 32; (16) SEQ ID NOS: 37 and 39; (17) SEQ ID NOS: 30 and 33; (18) SEQ ID NOS: 30 and 34; (19) SEQ ID NOS: 35 and 32; (20) SEQ ID NOS: 35 and 33; (21) SEQ ID NOS: 35 and 34; (22) SEQ ID NOS: 36 and 32; (23) SEQ ID NOS: 36 and 33; (24) SEQ ID NOS: 36 and 34; and (25) SEQ ID NOS: 28 and 138.

Any set of primers can be used simultaneously in a multiplex reaction with one or more other primer sets, so that multiple amplicons are amplified simultaneously.

A probe for binding to a sequence encoding C. Difficile toxin B gene comprises at least one of the following probe sequences: SEQ ID NOS: 2, 14, 16, 19, 22, 27, 29, 31, and 38.

A PCR primer set for amplifying sequences encoding C. Difficile tcdA gene (toxin A) comprises at least one of the following sets of primer sequences: (1) SEQ ID NOS: 40 and 42; (2) SEQ ID NOS: 43 and 44; (3) SEQ ID NOS: 45 and 47; (4) SEQ ID NOS: 48 and 50; (5) SEQ ID NOS: 51 and 42; (6) SEQ ID NOS: 48 and 52; (7) SEQ ID NOS: 53 and 54; (8) SEQ ID NOS: 55 and 42; and (9) SEQ ID NOS: 55 and 57.

Any set of primers can be used simultaneously in a multiplex reaction with one or more other primer sets, so that multiple amplicons are amplified simultaneously.

A probe for binding to a sequence encoding C. Difficile toxin A gene comprises at least one of the following probe sequences: SEQ ID NOS: 41, 46, 49, and 56.

A PCR primer set for amplifying sequences encoding C. Difficile cdtB gene (binary toxin) comprises at least one of the following sets of primer sequences: (1) SEQ ID NOS: 58 and 60; (2) SEQ ID NOS: 58 and 62; (3) SEQ ID NOS: 63 and 65; (4) SEQ ID NOS: 66 and 67; and (5) SEQ ID NOS: 68 and 60.

Any set of primers can be used simultaneously in a multiplex reaction with one or more other primer sets, so that multiple amplicons are amplified simultaneously.

A probe for binding to a sequence encoding C. Difficile binary toxin gene comprises at least one of the following probe sequences: SEQ ID NOS: 59, 61, 64, and 69.

Primer sets for simultaneously amplifying sequences encoding the genes for toxin B, and/or toxin A, and/or binary toxin comprises a nucleotide sequence selected from the primer sets consisting of: Groups 1-129 and 184 of Table 4 (toxin B), Groups 130-138 of Table 5 (toxin A), and Groups 139-145 of Table 6 (binary toxin). Oligonucleotide probes for binding to the genes for toxin B, and/or toxin A, and/or binary toxin comprises a nucleotide sequence selected from the group consisting of: SEQ ID NOS: 2, 14, 16, 19, 22, 27, 29, 31, and 38 (toxin B probes), SEQ ID NOS: 41, 46, 49, and 56 (toxin A probes), and SEQ ID NOS: 59, 61, 64, and 69 (binary toxin probes).

### Other Embodiments

Other embodiments will be evident to those of skill in the art. It should be understood that the foregoing detailed description is provided for clarity only and is merely exemplary. The spirit and scope of the present invention are not limited to the above examples, but are encompassed by the following claims. The contents of all references cited herein are incorporated by reference in their entireties.

What is claimed is:

1. An isolated nucleic acid sequence comprising a sequence selected from the group consisting of: SEQ ID NOS: 1-69 and 138.

2. A method of hybridizing one or more isolated nucleic acid sequences comprising a sequence selected from the group consisting of: SEQ ID NOS: 1-69 and 138 to a C. Difficile sequence, comprising contacting one or more isolated nucleic acid sequences to a sample comprising the C. Difficile sequence under conditions suitable for hybridization.

3. The method of claim 2, wherein the C. Difficile sequence is a genomic sequence, a template sequence, a sequence derived from an artificial construct or an artificially synthesized sequence.

4. The method of claim 2, further comprising the isolation of nucleic acid sequences containing a C. Difficile sequence.

5. The method of claim 2, further comprising quantitating the hybridized C. Difficile sequence.

6. The method of claim 2, further comprising sequencing of the hybridized C. Difficile sequence.

7. The method of claim 2, further comprising monitoring and/or screening for the presence of the hybridized C. Difficile sequence.

8. A primer set comprising at least one forward primer selected from the group consisting of SEQ ID NOS: 1, 4, 6, 8, 10, 12, 13, 18, 21, 23, 24, 26, 28, 30, 35, 36, 37, 40, 43, 45, 48, 51, 53, 55, 58, 63, 66, and 68, and at least one reverse primer selected from the group consisting of SEQ ID NOS: 3, 5, 7, 9, 11, 15, 17, 20, 25, 32, 33, 34, 39, 42, 44, 47, 50, 52, 54, 57, 60, 62, 65, 67 and 138.

9. The primer set of claim 8, wherein the primer set is selected from the group consisting of: Groups 1-129 and 184 of Table 4, Groups 130-138 of Table 5, and Groups 139-145 of Table 6.

10. A method of producing a nucleic acid product, comprising contacting one or more isolated nucleic acid sequences selected from the group consisting of SEQ ID NOS: 1, 3, 4, 5, 6, 7, 8, 9, 10, 11, 12, 13, 15, 17, 18, 20, 21, 23, 24, 25, 26, 28, 30, 32, 33, 34, 35, 36, 37, 39, 40, 42, 43, 44, 45, 47, 48, 50, 51, 52, 53, 54, 55, 57, 58, 60, 62, 63, 65, 66, 67, 68 and 138 to a sample comprising a C. Difficile sequence under conditions suitable for nucleic acid polymerization.

11. The method of claim 10, wherein the nucleic acid product is an amplicon produced using at least one forward primer selected from the group consisting of SEQ ID NOS: 1, 4, 6, 8, 10, 12, 13, 18, 21, 23, 24, 26, 28, 30, 35, 36, 37, 40, 43, 45, 48, 51, 53, 55, 58, 63, 66, and 68, and at least one reverse primer selected from the group consisting of SEQ ID NOS: 3, 5, 7, 9, 11, 15, 17, 20, 25, 32, 33, 34, 39, 42, 44, 47, 50, 52, 54, 57, 60, 62, 65, 67 and 138.

12. The method of claim 2, wherein the C. Difficile sequences are selected from the group consisting of: toxin B, toxin A, and binary toxin.

13. The method of claim 10, further comprising a probe that hybridizes to the nucleic acid product.

14. The probe of claim 13, wherein the probe comprises a sequence selected from the group consisting of: SEQ ID NOS: 2, 14, 16, 19, 22, 27, 29, 31, 38, 41, 46, 49, 56, 59, 61, 64, and 69.

15. The probe of claim 13, wherein the probe is labeled with a detectable label selected from the group consisting of: a fluorescent label, a chemiluminescent label, a quencher, a radioactive label, biotin, mass tags and gold.

16. The method of claim 11, further comprising a set of probes that hybridize to the amplicon, wherein a first probe comprises a sequence selected from the group consisting of: SEQ ID NOS: 2, 14, 16, 19, 22, 27, 29, 31, and 38, and a second probe comprises a sequence selected from the group consisting of: SEQ ID NOS: 41, 46, 49, and 56.

17. The method of claim 11, further comprising a set of probes that hybridize to the amplicon, wherein a first probe comprises a sequence selected from the group consisting of: SEQ ID NOS: 2, 14, 16, 19, 22, 27, 29, 31, and 38, a second probe comprises a sequence selected from the group consisting of: SEQ ID NOS: SEQ ID NOS: 41, 46, 49, and 56, and a third probe comprises a sequence selected from the group consisting of: SEQ ID NOS: 59, 61, 64, and 69.

18. The set of probes of claim 16, wherein the first probe is labeled with a first detectable label and the second probe is labeled with a second detectable label.

19. The set of probes of claim 16, wherein the first probe and the second probe are labeled with the same detectable label.

20. The set of probes of claim 18, wherein the detectable labels are selected from the group consisting of: a fluorescent label, a chemiluminescent label, a quencher, a radioactive label, biotin, mass tags and gold.

21. The set of probes of claim 17, wherein the first probe is labeled with a first detectable label, the second probe is labeled with a second detectable label and the third probe is labeled with a third detectable label.

22. The set of probes of claim 17, wherein the first probe, the second probe and the third probe are labeled with the same detectable label.

23. The set of probes of claim 17, wherein the first probe and the third probe are labeled with a first detectable label, and the second probe is labeled with a second detectable label.

24. The set of probes of claim 17, wherein the first probe is labeled with a first detectable label, and the second probe and third probe are labeled with a second detectable label.

25. The set of probes of claim 21, wherein the detectable labels are selected from the group consisting of: a fluorescent label, a chemiluminescent label, a quencher, a radioactive label, biotin, mass tags and gold.

26. A method for detecting a C. Difficile sequence in a sample, comprising:
a) contacting the sample with at least one forward primer comprising a sequence selected from the group consisting of: SEQ ID NOS: 1, 4, 6, 8, 10, 12, 13, 18, 21, 23, 24, 26, 28, 30, 35, 36, 37, 40, 43, 45, 48, 51, 53, 55, 58, 63, 66, and 68, and at least one reverse primer comprising a sequence selected from the group consisting of: SEQ ID NOS: 3, 5, 7, 9, 11, 15, 17, 20, 25, 32, 33, 34, 39, 42, 44, 47, 50, 52, 54, 57, 60, 62, 65, 67 and 138 under conditions such that nucleic acid amplification occurs to yield an amplicon; and
b) contacting the amplicon with one or more probes comprising one or more sequences selected from the group consisting of: SEQ ID NOS: 2, 14, 16, 19, 22, 27, 29, 31, 38, 41, 46, 49, 56, 59, 61, 64, and 69 under conditions such that hybridization of the probe to the amplicon occurs;
wherein hybridization of the probe is indicative of C. Difficile in the sample.

27. The method of claim 26, wherein each of the one or more probes is labeled with a different detectable label.

28. The method of claim 26, wherein the one or more probes are labeled with the same detectable label.

29. The method of claim 26, wherein the sample is selected from the group consisting of: blood, serum, plasma, enriched peripheral blood mononuclear cells, urine, neoplastic or other tissue obtained from biopsies, cerebrospinal fluid, saliva, fluids collected from the ear, eye, mouth, and respiratory airways, sputum, stool, skin, gastric secretions, oropharyngeal swabs, nasopharyngeal swabs, throat swabs, rectal swabs, nasal aspirates, nasal wash, fecal material, renal tissue, and fluid therefrom including perfusion media, pure cultures of bacterial fungal isolates, fluids and cells obtained by the perfusion of tissues of both human and animal origin, and fluids and cells derived from the culturing of human cells, including human stem cells and human cartilage or fibroblasts, pure cultures of bacterial fungal isolates, and swabs or washes of environmental surfaces, or other samples derived from environmental surfaces.

30. The method of claim 26, wherein the sample is from a human.

31. The method of claim 26, wherein the sample is non-human in origin.

32. The method of claim 26, wherein the sample is derived from an inanimate object.

33. The method of claim 26, wherein the at least one forward primer, the at least one reverse primer and the one or more probes is selected from the group consisting of: Groups 1-129 and 184 of Table 4, Groups 130-138 of Table 5, and Groups 139-145 of Table 6.

34. The method of claim 26, further comprising quantitating a C. Difficile sequence in a sample.

35. A kit for detecting a C. Difficile sequence in a sample, comprising one or more probes comprising a sequence selected from the group consisting of: SEQ ID NOS: 2, 14, 16, 19, 22, 27, 29, 31, 38, 41, 46, 49, 56, 59, 61, 64, and 69.

36. The kit of claim 35, further comprising:
a) at least one forward primer comprising the sequence selected from the group consisting of: SEQ ID NOS: 1, 4, 6, 8, 10, 12, 13, 18, 21, 23, 24, 26, 28, 30, 35, 36, 37, 40, 43, 45, 48, 51, 53, 55, 58, 63, 66, and 68; and
b) at least one reverse primer comprising the sequence selected from the group consisting of: SEQ ID NOS: 3, 5, 7, 9, 11, 15, 17, 20, 25, 32, 33, 34, 39, 42, 44, 47, 50, 52, 54, 57, 60, 62, 65, 67 and 138.

37. The kit of claim 35, further comprising an internal control or a process control.

38. The kit of claim 35, further comprising reagents for quantitating, monitoring, screening and/or sequencing a C. Difficile sequence in the sample.

39. The kit of claim 35, wherein the one or more probes are labeled with different detectable labels.

40. The kit of claim 35, wherein the one or more probes are labeled with the same detectable label.

41. The kit of claim 35, wherein the at least one forward primer and the at least one reverse primer are selected from the group consisting of: Groups 1-129 and 184 of Table 4, Groups 130-138 of Table 5, and Groups 139-145 of Table 6.

42. A method of diagnosing a C. Difficile-associated colonization, condition, syndrome or disease, comprising:
a) contacting a sample with at least one forward and reverse primer set selected from the group consisting of: Groups 1-129 and 184 of Table 4, Groups 130-138 of Table 5, and Groups 139-145 of Table 6;
b) conducting an amplification reaction, thereby producing an amplicon; and
c) detecting the amplicon using one or more probes selected from the group consisting of: SEQ ID NOS: 2, 14, 16, 19, 22, 27, 29, 31, 38, 41, 46, 49, 56, 59, 61, 64, and 69;
wherein the detection of an amplicon is indicative of the presence of C. Difficile in the sample.

43. The method of claim 42, wherein the sample is selected from the group consisting of: blood, serum, plasma, enriched peripheral blood mononuclear cells, urine, neoplastic or other tissue obtained from biopsies, cerebrospinal fluid, saliva, fluids collected from the ear, eye, mouth, and respiratory airways, sputum, stool, skin, gastric secretions, oropharyngeal swabs, nasopharyngeal swabs, throat swabs, rectal swabs, nasal aspirates, nasal wash, fecal material, renal tissue, and fluid therefrom including perfusion media, pure cultures of bacterial fungal isolates, fluids and cells obtained by the perfusion of tissues of both human and animal origin, and fluids and cells derived from the culturing of human cells, including human stem cells and human cartilage or fibroblasts, pure cultures of bacterial fungal isolates, and swabs or washes of environmental surfaces, or other samples derived from environmental surfaces.

44. The method of claim 42, wherein the C. Difficile-associated colonization, condition, syndrome or disease is selected from the group consisting of: watery diarrhea, abdominal pain, inflamed colon (colitis), appendicitis, small bowel enteritis, reactive arthritis, cellulitis, necrotizing fasciitis, osteomyelitis, fever, blood or pus in the stool, nausea, dehydration, loss of appetite, and weight loss.

45. A kit for binding, amplifying and sequencing a C. Difficile sequence in a sample, comprising:
a) at least one forward primer comprising the sequence selected from the group consisting of: SEQ ID NOS: 1, 4, 6, 8, 10, 12, 13, 18, 21, 23, 24, 26, 28, 30, 35, 36, 37, 40, 43, 45, 48, 51, 53, 55, 58, 63, 66, and 68;
b) at least one reverse primer comprising the sequence selected from the group consisting of: SEQ ID NOS: 3, 5, 7, 9, 11, 15, 17, 20, 25, 32, 33, 34, 39, 42, 44, 47, 50, 52, 54, 57, 60, 62, 65, 67 and 138; and
c) reagents for the sequencing of amplified DNA fragments.

46. The kit of claim 45, further comprising reagents for quantitating, monitoring and/or screening a C. Difficile sequence in a sample.

47. A method of diagnosing a C. Difficile-associated colonization, condition, syndrome or disease, comprising contacting a denatured target from a sample with one or more probes comprising a sequence selected from the group consisting of:
SEQ ID NOS: 2, 14, 16, 19, 22, 27, 29, 31, 38, 41, 46, 49, 56, 59, 61, 64, and 69 under conditions for hybridization to occur;
wherein hybridization of the one or more probes to a denatured target is indicative of the presence of C. Difficile in the sample.

48. The method of claim 47, wherein the sample is selected from the group consisting of: blood, serum, plasma, enriched peripheral blood mononuclear cells, urine, neoplastic or other tissue obtained from biopsies, cerebrospinal fluid, saliva, fluids collected from the ear, eye, mouth, and respiratory airways, sputum, stool, skin, gastric secretions, oropharyngeal swabs, nasopharyngeal swabs, throat swabs, rectal swabs, nasal aspirates, nasal wash, fecal material, renal tissue, and fluid therefrom including perfusion media, pure cultures of bacterial fungal isolates, fluids and cells obtained by the perfusion of tissues of both human and animal origin, and fluids and cells derived from the culturing of human cells, including human stem cells and human cartilage or fibroblasts, pure cultures of bacterial fungal isolates, and swabs or washes of environmental surfaces, or other samples derived from environmental surfaces.

49. The method of claim 47, wherein the C. Difficile-associated colonization, condition, syndrome or disease is selected from the group consisting of: watery diarrhea, abdominal pain, inflamed colon (colitis), appendicitis, small bowel enteritis, reactive arthritis, cellulitis, necrotizing fasciitis, osteomyelitis, fever, blood or pus in the stool, nausea, dehydration, loss of appetite, and weight loss.

50. A method for identifying the causative agent of watery diarrhea by detecting one or more C. Difficile strains in a sample based on its gene(s)) coding for toxin(s), the method comprising:
a) contacting the sample with at least one forward primer comprising the sequence selected from the group consisting of: SEQ ID NOS: 1, 4, 6, 8, 10, 12, 13, 18, 21, 23, 24, 26, 28, 30, 35, 36, 37, 40, 43, 45, 48, 51, 53, 55, 58, 63, 66, and 68, and at least one reverse primer comprising the sequence selected from the group consisting of: SEQ ID NOS: 3, 5, 7, 9, 11, 15, 17, 20, 25, 32, 33, 34, 39, 42, 44, 47, 50, 52, 54, 57, 60, 62, 65, 67 and 138 under conditions such that nucleic acid amplification occurs to yield an amplicon; and
b) contacting the amplicon with one or more probes comprising one or more sequences selected from the group consisting of: SEQ ID NOS: 2, 14, 16, 19, 22, 27, 29, 31, 38, 41, 46, 49, 56, 59, 61, 64, and 69 under conditions such that hybridization of the probe to the amplicon occurs;
wherein the hybridization of the probe is indicative of C. Difficile in the sample.

51. The method of claim 50, wherein the C. Difficile gene(s) encoding toxin(s) are selected from the group consisting of: toxin B, and/or toxin A, and/or binary toxin.

52. A method for identifying the causative agent of colitis by detecting one or more C. Difficile strains in a sample based on its gene(s) coding for toxin(s), the method comprising:
a) contacting the sample with at least one forward primer comprising the sequence selected from the group consisting of: SEQ ID NOS: 1, 4, 6, 8, 10, 12, 13, 18, 21, 23, 24, 26, 28, 30, 35, 36, 37, 40, 43, 45, 48, 51, 53, 55, 58, 63, 66, and 68, and at least one reverse primer comprising the sequence selected from the group consisting of: SEQ ID NOS: 3, 5, 7, 9, 11, 15, 17, 20, 25, 32, 33, 34, 39, 42, 44, 47, 50, 52, 54, 57, 60, 62, 65, 67 and 138 under conditions such that nucleic acid amplification occurs to yield an amplicon; and
b) contacting the amplicon with one or more probes comprising one or more sequences selected from the group consisting of: SEQ ID NOS: 2, 14, 16, 19, 22, 27, 29, 31, 38, 41, 46, 49, 56, 59, 61, 64, and 69 under conditions such that hybridization of the probe to the amplicon occurs;
wherein the hybridization of the probe is indicative of C. Difficile in the sample.

53. The method of claim 52, wherein the C. Difficile(s) encoding toxin(s) are selected from the group consisting of: toxin B, and/or toxin A, and/or binary toxin.

54. A screening kit for binding, amplifying and sequencing a C. Difficile sequence, comprising:
a) at least one forward primer comprising the sequence selected from the group consisting of: SEQ ID NOS: 1, 4, 6, 8, 10, 12, 13, 18, 21, 23, 24, 26, 28, 30, 35, 36, 37, 40, 43, 45, 48, 51, 53, 55, 58, 63, 66, and 68;
b) at least one reverse primer comprising the sequence selected from the group consisting of: SEQ ID NOS: 3, 5, 7, 9, 11, 15, 17, 20, 25, 32, 33, 34, 39, 42, 44, 47, 50, 52, 54, 57, 60, 62, 65, 67 and 138; and
c) reagents for the sequencing of amplified DNA fragments.

55. The kit of claim 54, further comprising a probe having a sequence selected from the group consisting of: SEQ ID NOS: 2, 14, 16, 19, 22, 27, 29, 31, 38, 41, 46, 49, 56, 59, 61, 64, and 69.

56. The kit of claim 54, further comprising an internal control or a process control.

57. The kit of claim 54, wherein the internal control is a sequence comprising a target SEQ ID NO: 73.

58. The kit of claim 54, wherein the internal control is detected by a forward primer comprising SEQ ID NO: 70, a reverse primer comprising SEQ ID NO: 72, and a probe comprising SEQ ID NO: 71.

59. The kit of claim 54, wherein the process control is detected by a forward primer comprising a sequence selected from the group consisting of SEQ ID NOS: 74-76, 83-93, 127, 128, 131, 135-137, a reverse primer selected from the group consisting of SEQ ID NOS: 79, 80, 82, 96, 103-120, 122, 125, 126 and a probe selected from the group consisting of SEQ ID NOS: 77, 78, 81, 94, 95, 97-102, 121, 123, 124, 129, 130, 132-134.

60. The kit of claim 45, wherein the process control is detected by a forward primer comprising a sequence selected from the group consisting of SEQ ID NOS: 74-76, 83-93, 127, 128, 131, 135-137, a reverse primer selected from the group consisting of SEQ ID NOS: 79, 80, 82, 96, 103-120, 122, 125, 126 and a probe selected from the group consisting of SEQ ID NOS: 77, 78, 81, 94, 95, 97-102, 121, 123, 124, 129, 130, 132-134.

61. The kit of claim 37, wherein the process control is detected by a forward primer comprising a sequence selected from the group consisting of SEQ ID NOS: 74-76, 83-93, 127, 128, 131, 135-137, a reverse primer selected from the group consisting of SEQ ID NOS: 79, 80, 82, 96, 103-120, 122, 125, 126 and a probe selected from the group consisting of SEQ ID NOS: 77, 78, 81, 94, 95, 97-102, 121, 123, 124, 129, 130, 132-134.
